(19) United States
(12) Patent Application Publication
(10) Pub. No.: US 2014/0128352 A1
(43) Pub. Date: May 8, 2014

(54) COMPOUNDS AND METHODS FOR MODULATING MITOCHONDRIAL METABOLISM AND REACTIVE OXYGEN SPECIES PRODUCTION

(71) Applicant: Buck Institute for Research on Aging, Novato, CA (US)

(72) Inventors: Martin D. Brand, Novato, CA (US); Adam Orr, Novato, CA (US)

(73) Assignee: Buck Institute for Research on Aging, Novato, CA (US)

(21) Appl. No.: 14/033,212

(22) Filed: Sep. 20, 2013

Related U.S. Application Data
(60) Provisional application No. 61/703,720, filed on Sep. 20, 2012.

(51) Int. Cl. A61K 31/63; A61K 31/505; A61K 31/47; A61K 31/495; A61K 31/433; C12Q 1/32; A61K 31/502; A61K 31/19; A61K 31/18; A61K 31/4184; G01N 33/50; A61K 31/438; A61K 31/472

(52) U.S. Cl. CPC A61K 31/63; A61K 31/438; A61K 31/505; A61K 31/47; A61K 31/495; A61K 31/433; A61K 31/472; A61K 31/502; A61K 31/19; A61K 31/18; A61K 31/4184; G01N 33/50; C12Q 1/32
USPC 514/155; 514/278; 514/274; 514/311; 514/255.03; 514/363; 514/309; 514/248; 514/561; 514/603; 514/394; 435/26; 436/63

(57) ABSTRACT

Compounds that modulate mitochondrial reactive oxygen species (ROS) production are provided. The compounds may modulate ROS production at defined sites without also altering energy production. Methods of using and identifying such compounds are also provided.

### CROSS-REFERENCE TO RELATED APPLICATIONS

This application claims the benefit under 35 U.S.C. §119(e) of U.S. Provisional Application Ser. No. 61/703,720, filed Sep. 20, 2012, the contents of which are incorporated herein by reference in their entirety.

### STATEMENT REGARDING FEDERALLY SPONSORED RESEARCH OR DEVELOPMENT

This invention was made, in part, with government support under grant number R01AG33542, awarded by the National Institutes of Health (NIH). The government may have certain rights in the invention.

### BACKGROUND OF THE INVENTION

Mitochondrial reactive oxygen species (ROS) production is central to the free radical theory of aging (Harman) and implicated in the pathogenesis of virtually all age-associated diseases including cardiovascular disease, neurodegeneration, cancer, and diabetes (Finkel & Holbrook, 2000; Lin & Beal, 2006; Van Gall et al, 2006; Muller et al, 2007). The bulk of research on the roles of ROS in aging and disease has focused on two areas: defining the mechanisms and sites of ROS production under normal and pathogenic conditions and the development of broad-acting antioxidant therapies to prevent or reverse ROS-related damage. Considerable progress has been made in defining sites of production and it is generally accepted that at least eight distinct sites exist within mitochondria (Brand, 2010; Quinlan et al, in submission). Although ROS production from Complexes I, II, and III (CI, CII, and CIII) of the respiratory chain have been implicated in various (patho)physiological events (e.g. Parkinson's disease, cancer, and hypoxia respectively), the current inability to dissociate ROS production from energy production has made causal ascription to any site equivocal at best. Similarly, antioxidant research has yielded thousands of natural and man-made chemicals and numerous gene products that broadly modulate reactive molecules with varying selectivity and potency and in different cellular compartments. By acting post-production, these scavengers are inadequate for mechanistic studies in complex systems and are typically unsuitable even for assignment of a specific source to a given type of reactive species (e.g. superoxide from one location or compartment over another). These disadvantages may account in part for the setbacks experienced by antioxidant-based therapeutics in human patients. Therefore, from fundamental understanding of mechanisms to effective clinical therapeutics, there remains an urgent need to identify ways in which mitochondrial ROS production is involved in (patho)physiological processes as well as the means by which it can be modulated selectively.

Compounds that modulate mitochondrial ROS production are provided. The compounds in one aspect modulate mitochondrial ROS production at defined sites in isolated mitochondria without also altering energy production. Methods of using and identifying such compounds are also provided.

### BRIEF SUMMARY OF THE INVENTION

Compounds, and pharmaceutically acceptable salts thereof, that are capable of ROS inhibition are provided. Compounds that selectively inhibit ROS inhibition are particularly provided. In one aspect, compounds are provided that selectively inhibit ROS production at one of the ROS production sites IQ, IF, IIIQ_{O}, SDH or GPDH to a greater extent than they reduce ROS production at the remaining ROS production sites IQ, IF, IIIQ_{O}, SDH and GPDH. In another variation, compounds are provided that reduce ROS production from one or more sites of ROS production and reduce membrane potential by no more than 4%. In another variation, compounds are provided that selectively reduces ROS production from a single site of ROS production and reduces membrane potential by no more than 4%.

Methods of using the compounds provided herein are also described. In one variation, a method of reducing oxidative stress in an individual in need thereof is provided, comprising administering to the individual an effective amount of a compound detailed herein, or a pharmaceutically acceptable salt thereof (e.g., a compound of Table 1 or of any of Formulas I, II, and III or a pharmaceutically acceptable salt thereof). Also provided is a method of inhibiting ROS production in an individual in need thereof, comprising administering to the individual an effective amount of a compound detailed herein, or a pharmaceutically acceptable salt thereof (e.g., a compound of Table 1 or of any of Formulas I, II, and III or a pharmaceutically acceptable salt thereof). In one aspect of the methods (e.g., methods of reducing oxidative stress or inhibiting ROS production), the method is carried out with a selective inhibitor of ROS production that affects one of the sites of ROS production IQ, IF, IIIQ_{O}, SDH or GPDH to a greater extent than it reduces ROS production at the remaining ROS production sites IQ, IF, IIIQ_{O}, SDH and GPDH. In one variation, the compound selectively reduces ROS production from one of the sites of ROS production by at least 20% and reduces ROS production at the remaining ROS sites by no more than 10%. In one variation, the compound selectively reduces ROS production from one of the sites of ROS production by at least 18% and reduces ROS production at the remaining ROS sites by no more than 12%. In one variation, the compound selectively reduces ROS production from one of the sites of ROS production at least about 2-fold or greater than its ability to reduce ROS production at the remaining ROS production sites. In one variation, the compound selectively reduces ROS production from one of the sites of ROS production at least about 1.5-fold or greater than its ability to reduce ROS production at the remaining ROS production sites. In one aspect, the compound selectively reduces ROS production at ROS production site IQ over ROS production sites IF, IIIQ_{O}, SDH and GPDH. In another aspect, the compound selectively reduces ROS production at ROS production site IF over ROS production sites IQ, IIIQ_{O}, SDH and GPDH. In another aspect, the compound selectively reduces ROS production at ROS production site IIIQ_{O} over ROS production sites IQ, IF, SDH and GPDH. In another aspect, the compound selectively reduces ROS production at ROS production site SDH over ROS production sites IQ, IF, IIIQ_{O} and GPDH. In another aspect, the compound selectively reduces ROS production at ROS production site GPDH over ROS production sites IQ, IF, IIIQ_{O} and SDH. In one variation, the compound reduces ROS production from one or more sites of ROS production and reduces membrane potential by no more than 4%. In another variation, the compound selectively reduces ROS production from a single site of ROS production and reduces membrane potential by no more than 4%.

In one variation, the individual in need of a reduction of ROS production has or is suspected of having a disease or condition in which ROS is implicated. In some variations, the disease or condition is selected from the group consisting of atherosclerosis, heart disease, heart failure, hypertension, UV damage to skin, sepsis, diabetes, Alzheimer's disease, Parkinson's disease, toxin-induced parkinsonism, Huntington's disease, Wilson's disease, Friedreich's Ataxia, Kearns-Sayre syndrome, Leigh syndrome, Leber hereditary optic neuropathy, mitochondrial myopathy, cardiomyopathy, deafness, mood disorders, movement disorders, dementia, Amyotropic Lateral Sclerosis, Multiple Sclerosis, tardive dyskinesia, brain injury, schizophrenia, epilepsy, AIDS dementia, endothelial nitroglycerin tolerance, adriamycin toxicity, kidney damage in type I diabetes, kidney preservation ex vivo, cocaine toxicity, alcohol fatty liver disease, fatty liver disease, liver inflammation in hepatitis C virus patients, neuroprotection, immobilization-induced muscle atrophy, skeletal muscle burn injury, cancer, inflammation and ischemic-reperfusion injury in stroke, heart attack and during organ transplantation and surgery. In individual may also or alternatively be at or over the age of any one of 40, 45, 50, 55, 60, 65, 70, 75, 80 years old.

Another aspect of the present invention is a pharmaceutical composition comprising a pharmaceutically acceptable carrier and a compound provided herein, or a pharmaceutically acceptable salt thereof (e.g., a compound of Table 1 or pharmaceutically acceptable salt thereof). It is also understood that the pharmaceutical composition could comprise a pharmaceutically acceptable carrier and a compound of any of Formulas I, II, and III, or a pharmaceutically acceptable salt thereof.

Also provided is a method of identifying a compound capable of: (1) selectively inhibiting ROS production at one of the ROS production sites IQ, IF, IIIQ_{O}, SDH, or GPDH to a greater extent than the remaining ROS production sites IQ, IF, IIIQ_{O}, SDH, and GPDH and (2) decreasing mitochondrial membrane potential by no more than 4% relative to untreated mitochondria, wherein the method comprises the steps of contacting a cell or purified mitochondria with a compound and measuring ROS production at ROS production sites IQ, IF, IIIQ_{O}, SDH and GPDH and measuring membrane potential. In one variation, the compound decreases ROS production from one of the ROS production sites IQ, IF, IIIQ_{O}, SDH, or GPDH by about or at least about any of 15% or 20% or 25% or 30% or 35% or 40% or 45% or 50% or more and decreases ROS production from the remaining ROS production sites IQ, IF, IIIQ_{O}, SDH, and GPDH by less than about 10%. In a further variation, the compound decreases ROS production from one of the ROS production sites IQ, IF, IIIQ_{O}, SDH, or GPDH by about or at least about any of 15% or 20% or 25% or 30% or 35% or 40% or 45% or 50% or more and decreases ROS production from the remaining ROS production sites IQ, IF, IIIQ_{O}, SDH, and GPDH by less than about 10% while decreasing mitochondrial membrane potential by no more than 4% relative to untreated mitochondria. In

Also provided is a method of identifying a compound capable of selectively inhibiting ROS production at one of the ROS production sites IQ, IF, IIIQ_{O}, SDH, or GPDH to a greater extent than the remaining ROS production sites IQ, IF, IIIQ_{O}, SDH, and GPDH, wherein the method comprises the steps of contacting a cell or purified mitochondria with a compound and measuring ROS production at ROS production sites IQ, IF, IIIQ_{O}, SDH and GPDH.

Also provided is a method of identifying a compound capable of selectively inhibiting enzymatic activity of one of the ROS production sites IQ, IF, IIIQ_{O}, SDH, or GPDH to a greater extent than the remaining ROS production sites IQ, IF, IIIQ_{O}, SDH, and GPDH, wherein the method comprises the steps of contacting a cell or purified mitochondria with a compound and measuring ROS production at ROS production sites IQ, IF, IIIQ_{O}, SDH and GPDH.

Also provided is a method of identifying a compound capable of: (1) inhibiting ROS production at one or more ROS production sites IQ, IF, IIIQ_{O}, SDH, or GPDH and (2) decreasing mitochondrial membrane potential by no more than 4% relative to untreated mitochondria, wherein the method comprises the steps of contacting a cell or purified mitochondria with a compound and measuring ROS production at ROS production sites IQ, IF, IIIQ_{O}, SDH and GPDH and measuring membrane potential.

### BRIEF DESCRIPTION OF THE DRAWINGS

FIG. 1 illustrates a scheme for the identification of site-specific ROS inhibitors. FIG. 1A illustrates that multiple sites of ROS production exist in the mitochondrial electron transport chain and can be reliably induced to generate maximal ROS through the use of specific substrate and inhibitor combinations. FIG. 1B illustrates an array of six high throughput assays to screen in parallel for compounds that inhibit ROS production from a single site in mitrochondria (sites IQ, IF, IIIQo, SDH and mGPDH) without altering the ability of mitochondria to maintain the membrane potential that drives energy production (ΔΨm). FIG. 1C illustrates normalized results for compound effects on IQ-ROS production. Out of 3,200 compounds tested, over 100 inhibited IQ-ROS projection by more than 20% and 13 (highlighted as darkened circles) do not significantly alter ROS production from the other 4 sites tested (see FIG. 1D; other sites not shown) or alter membrane potential (see FIG. 1E). FIG. 1D illustrates compound effects on IIIQo. FIG. 1E illustrates compound effects on mitochondrial membrane potential. FIG. 1F illustrates data for site selective ROS suppressors for each of the five sites. FIG. 1G illustrates how further analysis may be performed for selectivity and activity in both isolated and intact systems.

FIG. 2A demonstrates the rate of H₂O₂ production and illustrates that Compound No. 4 suppresses up to 65% of the ROS conservatively attributed to site IQ as defined by the complex I inhibitor rotenone. Means +/−SE, n=3. FIG. 2B demonstrates that Compound No. 4 is distinct from both the uncoupler FCCP and the complex I inhibitor rotenone in its effect on ROS production and the rate of reduction of matrix NADH via complex I.

FIG. 3A demonstrates that Compound No. 19 selectively lowers ROS production from site IF. FIGS. 3B and 3C illustrate measurements of mitochondrial respiration on complex I substrates (FIG. 3B) or complex II substrates (FIG. 3C) and indicate that Compound No. 19 inhibits ADP-stimulated respiration with complex I substrates.

FIGS. 4A and 4B demonstrate that Compound No. 32 selectively lowers ROS production from site IF (FIG. 4A) without any significant effects on membrane potential (ΔΨm) (FIG. 4B). FIG. 4C illustrates that Compound No. 32 also lowers the amount of ROS production from site IF as the matrix NADH pool becomes strongly reduced. FIGS. 4D and 4E provide measurements of mitochondrial respiration on complex I substrates (FIG. 4D) or complex II substrates (FIG. 4E) to reveal that Compound No. 32 increases ADP-stimulated respiration specifically with complex I substrates.

FIG. 5A demonstrates that Compound No. 34 selectively lowers ROS production from site IIIQo independent of substrate concentration. FIG. 5B illustrates that Compound No. 34 increases ΔΨm at 8 μM but collapses ΔΨm above this concentration. FIGS. 5C, 5D and 5E provide measurement of mitochondrial respiration and show no effect at 8 μM but signs of uncoupling at 80 μM for all substrates. FIG. 5F illustrates that Compound No. 37 lowers ROS from site IIIQo independent of substrate concentration but also lowers site IQ ROS with subsaturating succinate. FIG. 5G shows that unlike Compound No. 34, Compound No. 37 does not show signs of uncoupling state 2 respiration at high concentrations.

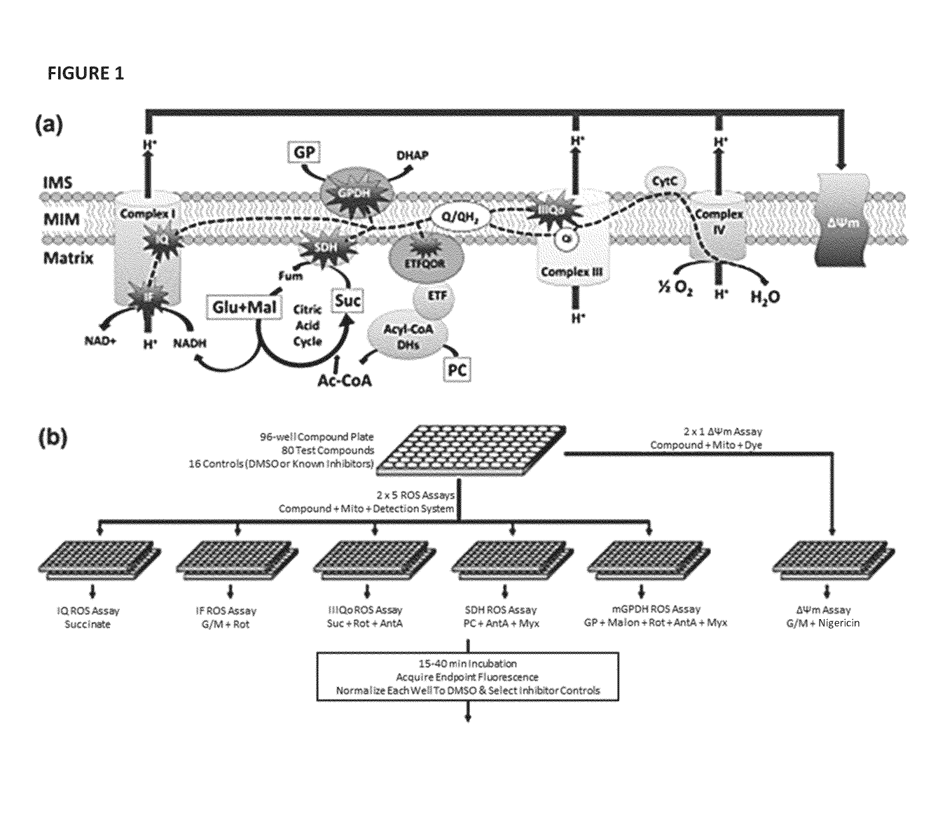

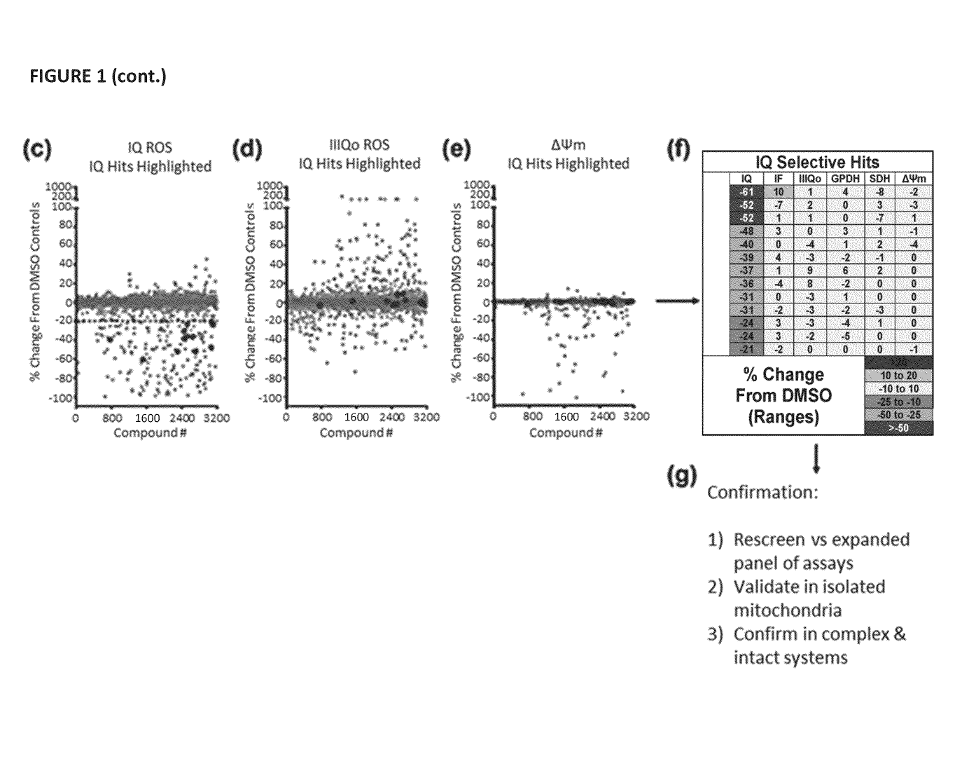

### DETAILED DESCRIPTION OF THE INVENTION

Overview of the Methods

The compounds described herein may be used in a method to reduce reactive oxygen species (ROS) production from one or more sites of ROS production. In some aspects of the invention, the compounds specifically inhibit ROS production at a single site of ROS production, while having minimal effects on ROS production from the remaining sites. In some aspects of the invention, the compounds inhibit ROS production, while having minimal effects on mitochondrial membrane potential (ΔΨm). In some aspects of the invention, the compounds specifically inhibit ROS production at a single site of ROS production, while having minimal effects on ROS production from the remaining sites and minimal effects on mitochondrial membrane potential (ΔΨm).

High-throughput assays for the identification of compounds that modulate ROS production at defined sites in isolated mitochondria without also altering energy production are also described. The assays identify site specific modulators of ROS production while also revealing less specific effectors like broad-acting antioxidants and various inhibitors of mitochondrial bioenergetics. Accordingly, compounds that discriminate between unwanted electron leak onto oxygen (ROS production) at specific sites within the electron transport chain without altering the normal energy-coupled electron and proton fluxes across the inner mitochondrial membrane may be identified. The assays adapt standard fluorescence based assays of mitochondrial ROS production using the dye Amplex UltraRed (Invitrogen) and ΔΨm using the potentiometric dye TMRM (Invitrogen) to a high throughput microplate format. A core set of five ROS and one ΔΨm assays for robust detection of functional modulation in freshly isolated skeletal muscle mitochondria are provided. Five major sites of ROS production (site IQ, IF, IIIQo, SDH, and mGPDH) may be targeted separately by varying the substrates and inhibitors added to a common assay mixture. A counterscreen to monitor ΔΨm may be run in parallel to eliminate compounds that are likely general inhibitors or uncouplers of normal mitochondrial energy production.

In one aspect, compounds are tested at 2.5 μM in duplicate against all assays. Endpoint fluorescence is normalized to DMSO and known mitochondrial inhibitor control wells included on each plate. Positive hits in each ROS assay may be initially filtered by applying a threshold of, e.g., 15% or 18% or 20% or more reduction in that assay. Each ROS assay may be employed as a counterscreen against the others while also eliminating compounds that altered ΔΨm in the TMRM-based counterscreen. Therefore, filtered hits may be subsequently assessed to eliminate those that altered the other ROS assays by more than e.g., 20% or 18% or 15% or ΔΨm by more than, e.g., 10% or 5% or 4%.

In one aspect, compounds that are selective inhibitors of ROS production from a single site of ROS production decrease ROS production from one of the ROS production sites IQ, IF, IIIQO, SDH and GPDH by greater than 18%, while affecting ROS production from the remaining sites of ROS production by less than 12%.

Sites of ROS Production:

Complex I of the respiratory chain can generate ROS from two distinct sites: the ubiquinone binding site and the flavin mononucleotide site. Inhibition of complex I activity by rotenone and the neurotoxin MPP+ has been linked to parkinsonism in both rodents and humans suggesting a possible link between dysfunctional complex I, ROS production, and neurodegeneration. Compounds that are capable of inhibiting ROS production from complex I may therefore be useful in therapy.

Ubiquinone Binding Site of Complex I (IQ)

To specifically analyze ROS production from IQ, 5 mM succinate may be used as the substrate to supply electrons to the respiratory chain. IQ ROS production is exceptionally sensitive to changes in the proton motive force (PMF) across the mitochondrial inner membrane (PMF=ΔΨm+ΔpH). Therefore, a conservative threshold for the ΔΨm assay when evaluating selectivity of hits in the IQ ROS assay may be utilized.

Electron leak from site IQ is best characterized during reverse transport from a reduced Q-pool to matrix NAD+ via CI in the presence of a strong PMF (Brand M D, 2010). Experimentally, conditions that favor IQ ROS production are considered far removed from physiology leading many to dismiss its relevance despite its capacity for high rates (Brand M D, 2010; Quinlan et al, 2012). However, even when provided with lower concentrations of both glutamate (to feed electrons forward through CI) and succinate (to feed electrons in reverse), respiring mitochondria still produce significant levels of rotenone-sensitive ROS (i.e. IQ ROS) (Muller et al, BJ, 2008). Further, comparative analyses show an inverse relationship between maximal ROS production from site IQ (but not site IF) and maximum life span across diverse vertebrate species (Lambert, A. et al. (2007) Low rates of hydrogen peroxide production by isolated heart mitochondria associate with long maximum lifespan in vertebrate homeotherms. Aging Cell. 6(5):607-18; Lambert, A. et al. (2010) Low complex I content explains the low hydrogen peroxide production rate of heart mitochondria from the long-lived pigeon, Columba livia. Aging Cell. 9(1):78-91.). Therefore, selective modulators of IQ ROS would offer unique opportunities to probe the putative role of mitochondrial ROS production in normal and pathological processes. Compounds capable of inhibiting ROS production from IQ include those listed in Table 2.

Flavin Binding Site of Complex I (IF)

To specifically analyze ROS production from IF, the substrate solution to supply electrons to the respiratory chain may comprise 5 mM glutamate, 5 mM malate, and 4 μM rotenone. Site IF produces ROS at a rate proportional to the reduction state of the NADH pool in the mitochondrial matrix (Treberg, J. et al. (2011) Evidence for Two Sites of Superoxide Production by Mitochondrial NADH-Ubiquinone Oxidoreductase (Complex I). J. Biol. Chem. 286(36):31361-72; Quinlan refs). Blockade of site IQ with the pesticide rotenone can increase ROS production from site IF by preventing oxidation of the flavin. Maximal ROS production from the flavin binding site of complex I (site IF) is relatively low compared to sites IQ and IIIQo and this may lead to higher variability in this assay and subsequently a higher false positive rate of hit calling in the original screen. Compounds capable of inhibiting ROS production from IF include those listed in Table 3.

Outer Q-Binding Site of Complex III (Site IIIQo)

ROS production attributed to Complex III has been linked to hypoxic signaling, cellular differentiation, and tumor growth. Therefore, compounds that are capable of inhibiting ROS production from complex III may be useful in therapy. To specifically analyze ROS production from IIIQo, the substrate solution to supply electrons to the respiratory chain may comprise 5 mM succinate, 4 μM rotenone, and 2.5 μM antimycin A. Site IIIQo produces ROS at high rates in the presence of the inhibitor antimycin A but has also recently been implicated as a source of ROS in mitochondria respiring on CI substrates in the absence of inhibitors (Quinlan et al, 2012). Half the superoxide produced from IIIQo is directed toward the cytosolic side of the inner mitochondrial membrane. This has led to speculation about the role of site IIIQo in cellular signaling during hypoxia, differentiation, and tumorogenesis although this remains contentious. Compounds capable of inhibiting ROS production from IIIQo include those listed in Table 4.

Complex II—Succinate Dehydrogenase (SDH)

Complex II ROS production has been implicated in cancer. Therefore, compounds that are capable of inhibiting ROS production from complex II may be useful in therapy. To specifically analyze ROS production from SDH, the substrate solution to supply electrons to the respiratory chain may comprise 15 μM palmitoyl carnitine, 2.5 μM antimycin A, and 2 μM myxothiazol. Compounds capable of inhibiting ROS production from SDH included those listed in Table 5.

Glycerol-3-Phosphate Dehydrogenase (GPDH)

Currently, there are no known inhibitors of enzymatic activity of mitochondrial GPDH. Previously reported inhibitors, like small metabolites, display varying potency but none are selective or cell permeant. To specifically analyze ROS production from GPDH, the substrate solution to supply electrons to the respiratory chain may comprise 25 mM glycerol phosphate, 4 μM rotenone, 2.5 μM antimycin A, 1 mM malonate and 2 μM myxothiazol. Compounds capable of inhibiting ROS production from GPDH include those listed in Table 6.

Compounds of the Invention:

Compounds according to the invention are detailed herein, including in the Brief Summary of the Invention and the appended claims. Thus, compounds of the invention include any compounds detailed herein, or salts thereof, such a pharmaceutically acceptable salts thereof. The invention includes the use of all of the compounds described herein, including any and all stereoisomers or salts of the compounds described as inhibitors of reactive oxygen species production. Further methods of using the compounds of the invention are detailed throughout.

The invention includes all compounds of Table 1 and pharmaceutically acceptable salts thereof.

TABLE 1

| | |
|---|---|
| 1. | 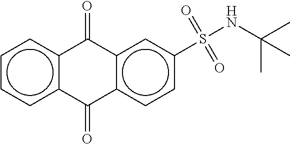 |
| 2. | 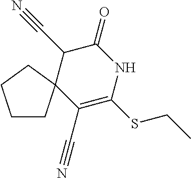 [SMILES: CCSC1=C(C#N)C2(CCCC2)C(C#N)C(=O)N1] |
| 3. | 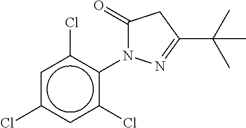 |
| 4. | 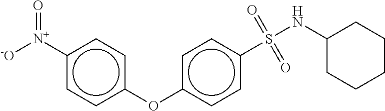 |
| 5. | 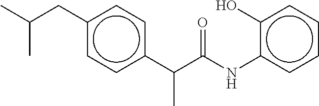 |
| 6. | 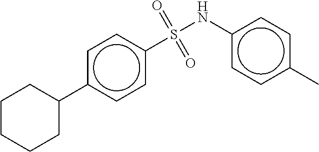 |
| 7. | 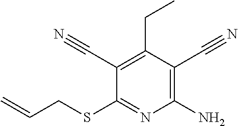 [SMILES: C=CCSc1nc(N)c(C#N)c(CC)c1C#N] |
| 8. | 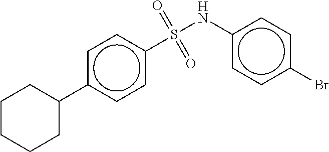 |
| 9. | 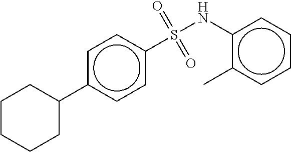 |
| 10 | 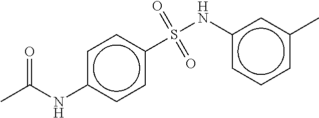 |
| 11. | 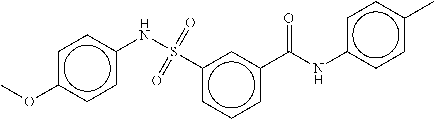 |
| 12. | 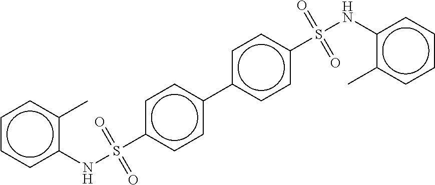 |
| 13. | 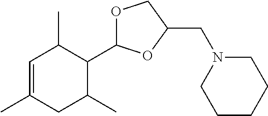 [SMILES: CC1=CC(C)C(C2OCC(CN3CCCCC3)O2)C(C)C1] |
| 14. | 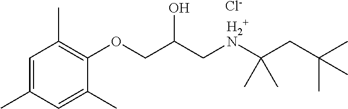 |
| 15. | 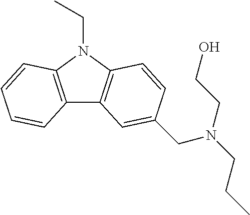 [SMILES: CCCN(CCO)Cc1ccc2c(c1)c1ccccc1n2CC] |
| 16. | 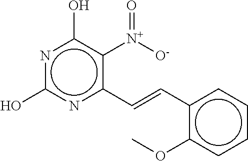 |
| 17. | 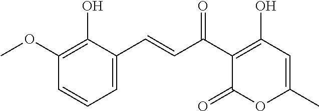 |
| 18. | 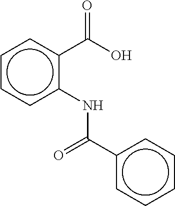 |
| 19. | 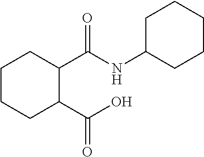 [SMILES: O=C(O)C1CCCCC1C(=O)NC1CCCCC1] |
| 20. | 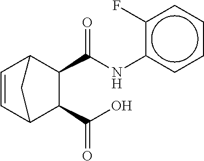 |
| 21. | 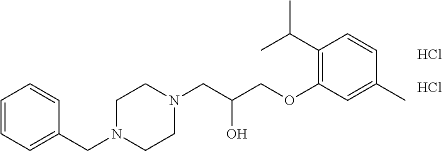 [SMILES: Cc1ccc(C(C)C)c(OCC(O)CN2CCN(Cc3ccccc3)CC2)c1 ; Cl ; Cl] |
| 22. | 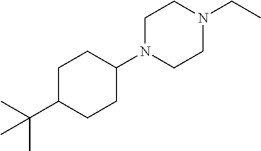 [SMILES: CCN1CCN(C2CCC(C(C)(C)C)CC2)CC1] |
| 23. | 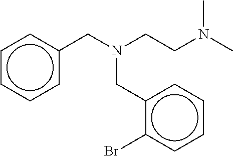 |
| 24. | 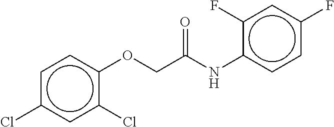 |
| 25. | 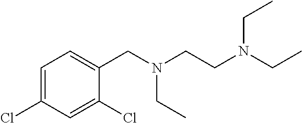 [SMILES: CCN(CC)CCN(CC)Cc1ccc(Cl)cc1Cl] |
| 26. | 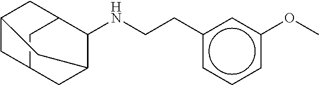 |
| 27. | 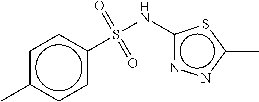 |
| 28. | 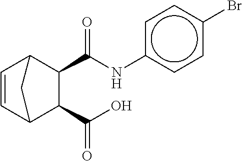 |
| 29. | 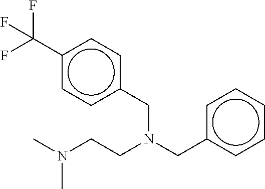 |
| 30. | 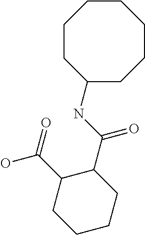 [SMILES: O=C(O)C1CCCCC1C(=O)NC1CCCCCCC1] |
| 31. | 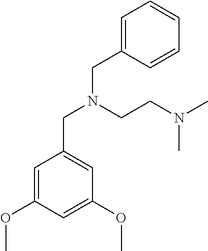 [SMILES: COc1cc(CN(CCN(C)C)Cc2ccccc2)cc(OC)c1] |
| 32. | 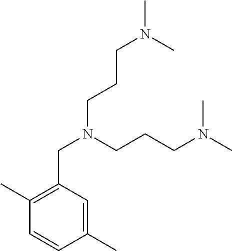 |
| 33. | 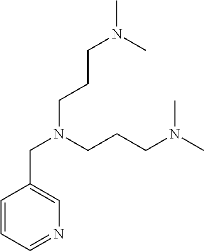 [SMILES: CN(C)CCCN(CCCN(C)C)Cc1cccnc1] |
| 34. | 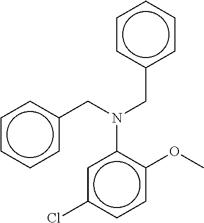 |
| 35. | 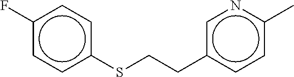 |
| 36. | 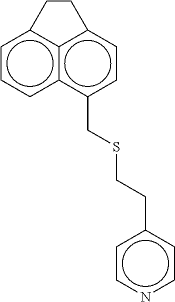 |
| 37. | 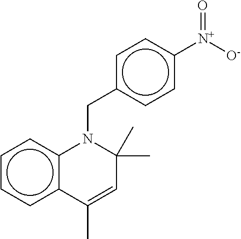 |
| 38. | 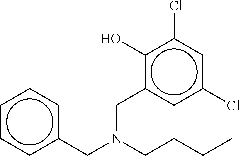 |
| 39. | 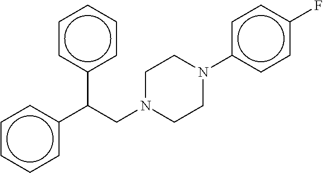 |
| 40. | 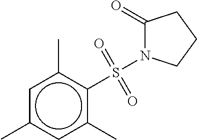 |
| 41. | 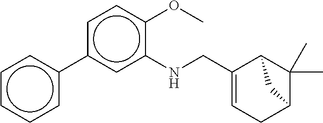 |
| 42. | 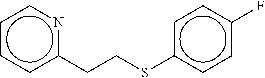 |
| 43. | 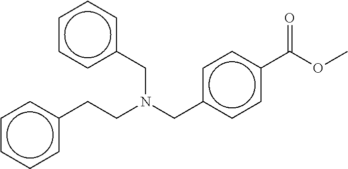 |
| 44. | 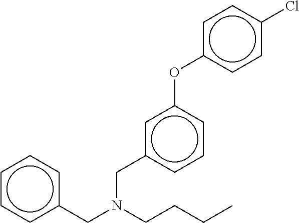 |
| 45. | 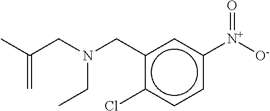 |
| 46. | 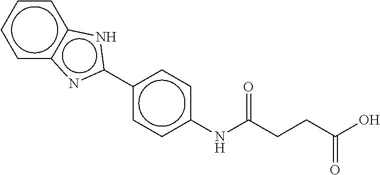 |
| 47. | 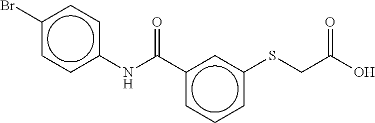 |
| 48. | 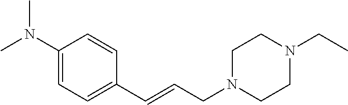 [SMILES: CCN1CCN(C/C=C/c2ccc(N(C)C)cc2)CC1] |
| 49. | 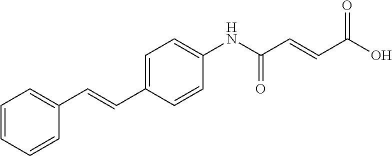 [SMILES: O=C(O)/C=C/C(=O)Nc1ccc(/C=C/c2ccccc2)cc1] |
| 50. | 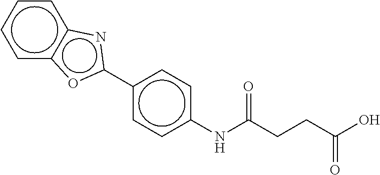 |
| 51. | 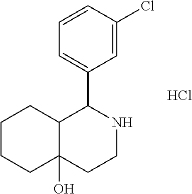 [SMILES: OC12CCCCC1C(c1cccc(Cl)c1)NCC2 ; Cl] |
| 52. | 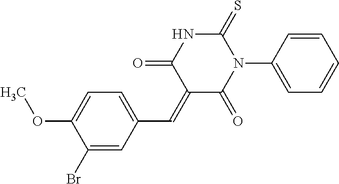 [SMILES: COc1ccc(/C=C2\C(=O)NC(=S)N(c3ccccc3)C2=O)cc1Br] |
| 53. | 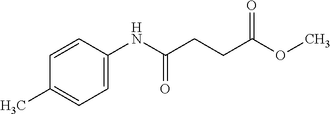 |
| 54. | 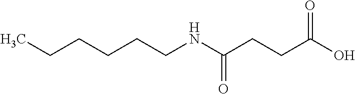 |
| 55. | 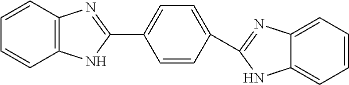 [SMILES: c1ccc2[nH]c(-c3ccc(-c4nc5ccccc5[nH]4)cc3)nc2c1] |
| 56. | 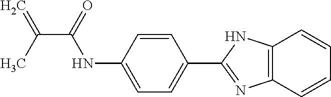 [SMILES: C=C(C)C(=O)Nc1ccc(-c2nc3ccccc3[nH]2)cc1] |
| 57. | 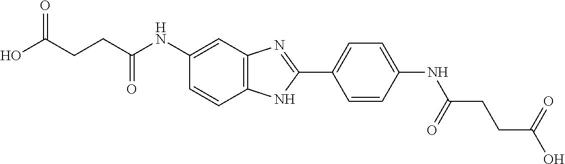 [SMILES: O=C(O)CCC(=O)Nc1ccc(-c2nc3cc(NC(=O)CCC(=O)O)ccc3[nH]2)cc1] |
| 58. | 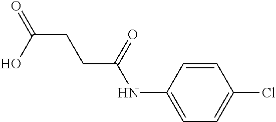 [SMILES: O=C(O)CCC(=O)Nc1ccc(Cl)cc1] |
| 59. | 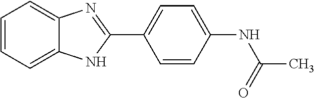 [SMILES: CC(=O)Nc1ccc(-c2nc3ccccc3[nH]2)cc1] |
| 60. | 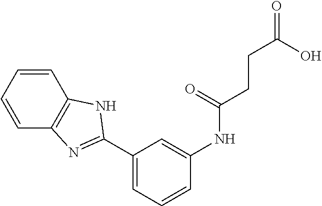 [SMILES: O=C(O)CCC(=O)Nc1cccc(-c2nc3ccccc3[nH]2)c1] |
| 61. | 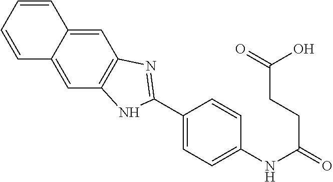 [SMILES: O=C(O)CCC(=O)Nc1ccc(-c2nc3cc4ccccc4cc3[nH]2)cc1] |
| 62. | 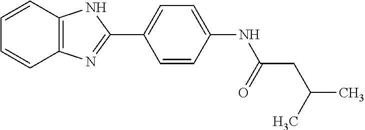 [SMILES: CC(C)CC(=O)Nc1ccc(-c2nc3ccccc3[nH]2)cc1] |
| 63. | 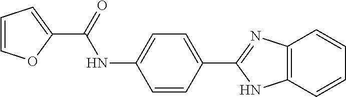 [SMILES: O=C(Nc1ccc(-c2nc3ccccc3[nH]2)cc1)c1ccco1] |
| 64. | 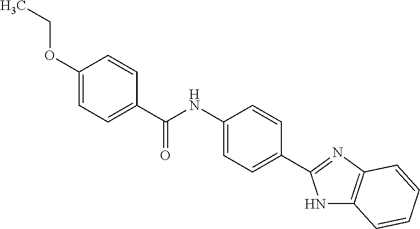 [SMILES: CCOc1ccc(C(=O)Nc2ccc(-c3nc4ccccc4[nH]3)cc2)cc1] |
| 65. | 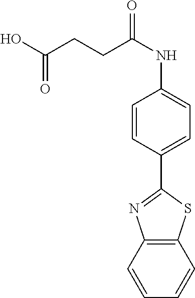 [SMILES: O=C(O)CCC(=O)Nc1ccc(-c2nc3ccccc3s2)cc1] |
| 66. | 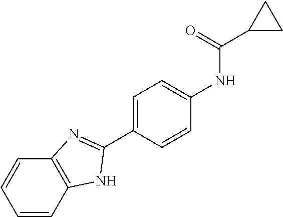 [SMILES: O=C(Nc1ccc(-c2nc3ccccc3[nH]2)cc1)C1CC1] |
| 67. | 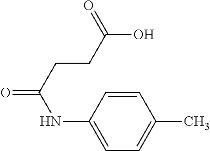 |
| 68. | 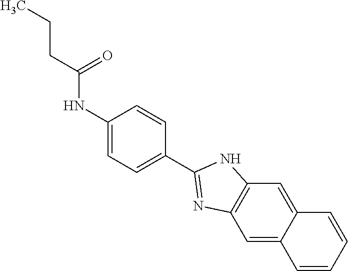 [SMILES: CCCC(=O)Nc1ccc(-c2nc3cc4ccccc4cc3[nH]2)cc1] |
| 69. | 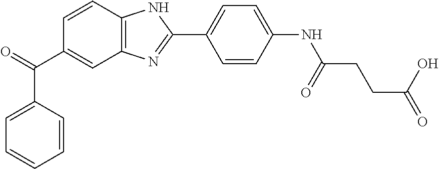 [SMILES: O=C(O)CCC(=O)Nc1ccc(-c2nc3cc(C(=O)c4ccccc4)ccc3[nH]2)cc1] |
| 70. | 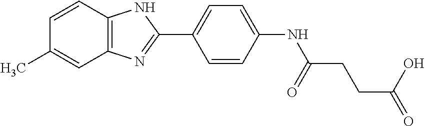 [SMILES: Cc1ccc2[nH]c(-c3ccc(NC(=O)CCC(=O)O)cc3)nc2c1] |
| 71. | 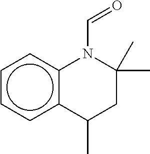 |
| 72. | 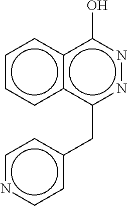 |
| 73. | 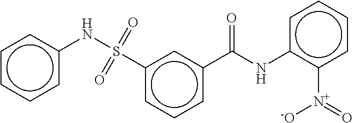 |
| 74. | 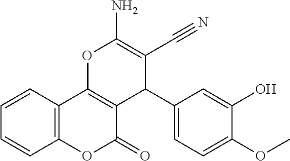 [SMILES: COc1ccc(C2C(C#N)=C(N)Oc3c2c(=O)oc2ccccc32)cc1O] |
| 75. | 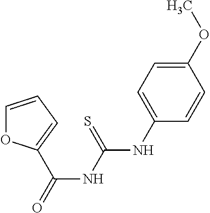 [SMILES: COc1ccc(NC(=S)NC(=O)c2ccco2)cc1] |
| 76. | 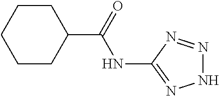 [SMILES: O=C(Nc1nn[nH]n1)C1CCCCC1] |
| 77. | 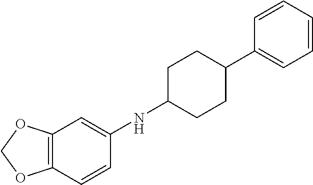 [SMILES: c1ccc(C2CCC(Nc3ccc4c(c3)OCO4)CC2)cc1] |
| 78. | 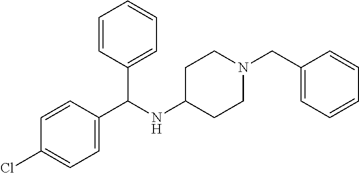 [SMILES: Clc1ccc(C(NC2CCN(Cc3ccccc3)CC2)c2ccccc2)cc1] |
| 79. | 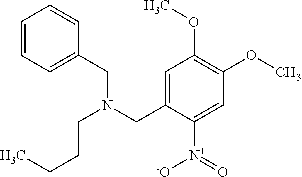 |
| 80. | 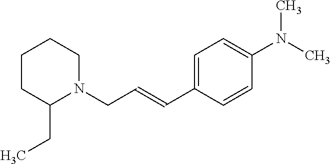 [SMILES: CCC1CCCCN1C/C=C/c1ccc(N(C)C)cc1] |
| 81. | 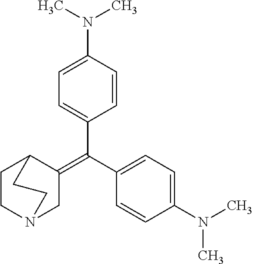 [SMILES: CN(C)c1ccc(C(=C2CN3CCC2CC3)c2ccc(N(C)C)cc2)cc1] |
| 82. | 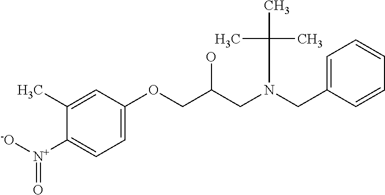 |
| 83. | 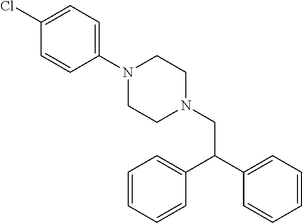 [SMILES: Clc1ccc(N2CCN(CC(c3ccccc3)c3ccccc3)CC2)cc1] |
| 84. | 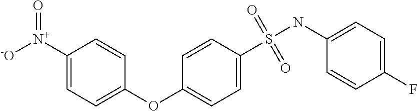 |
| 85. | 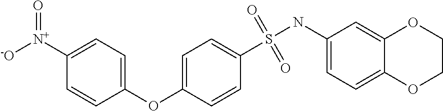 |

All of the compounds of Table 1 are commercially available, e.g. from ChemBridge Corporation, 11199 Sorrento Valley Rd, Suite 206, San Diego, Calif. 92121.

The invention also includes compounds of the Formula (I):

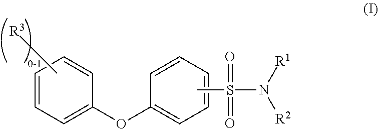

wherein:
R¹ and R² are independently selected from the group consisting of H, unsubstituted C₁-C₆ alkyl, —N═C(H)(aryl), —NH₂, unsubstituted aryl, substituted aryl or may be taken together with the nitrogen to which they are attached to form a nitrogen containing heterocyclic ring that may contain one additional heteroatom; and
R³ is selected from the group consisting of nitro, halo and C₁-C₆alkoxy;
   and pharmaceutically acceptable salts thereof.

The invention also includes compounds of the Formula (II):

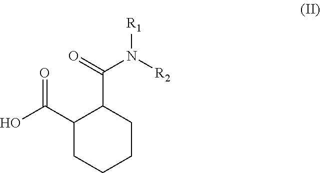

wherein R¹ and R² are independently H or a C₃-C₈cycloalkyl,
and pharmaceutically acceptable salts thereof.

The invention also includes compounds of the Formula (III):

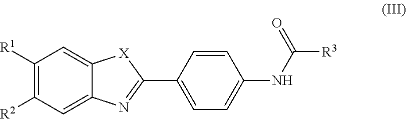

wherein:

R¹ and R² are independently selected from the group consisting of H, unsubstituted C₁-C₆ alkyl, C(═O)phenyl, phenyl, or may be taken together to form an aromatic ring;

R³ is selected from the group consisting of C₁-C₆ alkyl, carboxyl, and phenyl substituted with C₁-C₆ alkoxy; and

X is selected from the group NH, S and O,

and pharmaceutically acceptable salts thereof.

As used herein the term “alkyl” is intended to include linear, branched, or cyclic hydrocarbon structures and combinations thereof. Particular alkyl groups are those having 20 or fewer carbon atoms (a “C₁-C₂₀ alkyl”). More particular alkyl groups are those having 6 or fewer carbon atoms (“C₁-C₆ alkyl”). It is understood that the term “alkyl” includes cycloalkyl, such as a cycloalkyl having from 3 to 8 annular carbon atoms.

As used herein the term “alkoxy” refers to the group alkyl-O—

As used herein the term “aryl” refers to an unsaturated aromatic carbocyclic group or heterocyclic group of from 6 to 14 carbon atoms having a single ring or multiple condensed rings which condensed rings may or may not be aromatic provided that the point of attachment is aromatic.

“Carbonyl” refers to the group—C═O.

“Heterocycle” refers to a saturated or unsaturated group having a single ring or multiple condensed rings, from 1 to 10 carbon atoms and from 1 to 4 annular heteroatoms selected from nitrogen, sulfur or oxygen.

“Halo” includes fluoro, chloro, bromo and iodo.

### EXAMPLES

### Example 1

### Isolation of Mitochondria

Skeletal muscle mitochondria were isolated from 5-8 week old female Wistar rats (Harlan Laboratories). Minced muscle was homogenized in buffer containing bacterial protease Type VIII prior to differential centrifugation to separate mitochondria from other tissue components. Unless otherwise stated, freshly isolated muscle mitochondria were assayed in buffer containing 120 mM KCl, 5 mM HEPES, 1 mM EGTA, 0.3% (w/v) bovine serum albumin. Where indicated, CaCl₂ was added to yield a final free concentration of 250 nM calcium at pH 7.0 at 37° C. Total and free calcium concentrations were calculated using the Extended MaxChelator program at http://maxchelator.stanford.edu.

### Example 2

### Preparation of Plates for High-Throughput Screen for Unique Modulators of Mitochondrial Reactive Oxygen Species Production

Compounds (10 mM in DMSO) were randomly selected from a library of 24,000 compounds obtained from ChemBridge and curated by the lab of Robert Hughes at the Buck Institute. Master screening plates were prepared using a Biomek FX liquid handling workstation (Beckman) to transfer 1 μL of each compound into individual wells on 96-well storage plates (80 compounds per plate). Master plates were sealed with foil and stored at −80° C. Just prior to use, plates were brought to room temperature and diluted 300-fold with standard buffer (13.3X=33.3 μM). To monitor the performance of each assay throughout screening and to provide intra-plate normalization reference points, DMSO negative controls and known mitochondrial inhibitor positive controls were included on each Master compound plate. Eight DMSO wells and two wells each of the following inhibitor controls (prepared fresh at 13.3X in standard buffer) were included on each plate: FCCP, an uncoupler of the proton motive force and positive control for site IQ ROS (4 μM final); myxothiazol, a selective inhibitor of the outer ubiquinone binding site of complex III and positive control for site IQ and site IIIQo ROS (2 μM final); malonate, a competitive inhibitor of complex II/succinate dehydrogenase and positive control for site SDH ROS (10 mM final); and aspartate, a competitive substrate for mitochondrial metabolite carriers and positive control for site IF ROS (20 mM final).

### Example 3

### Preparation of Solution for the Detection of Reactive Oxygen Species

Total amounts of superoxide and H₂O₂ production were determined fluorometrically as H₂O₂, without distinguishing between them, in the presence of exogenous superoxide dismutase (25 units/mL final), horseradish peroxidase (1 unit/mL final), and Amplex UltraRed (25 μM final) as described (Affourtit, C. et al. (2012) Measurement of proton leak and electron leak in isolated mitochondria. Methods Mol. Biol. 810: 165-82). Just prior to use, a bulk 3.3X solution was made in standard buffer and distributed into a 2 mL deep well 96-well transfer plate.

### Example 4

### Identification of Inhibitors of the Ubiquinone Binding Site of Complex I

To assess the effects of a compound on ROS production from the ubiquinone binding site of complex I (IQ), Start Solution containing 5 mM succinate was distributed into standard 96-well transfer plates. A Biomek FX workstation was programmed to dispense solutions into black 96-well assay plates. Solutions were dispensed to all assay plates in the following order: ROS Detection solution described in Example 3, Mitochondria solution described in Example 1 to a final concentration of 0.1 mg/ml, and Compound dilutions described in Example 2. Duplicate assay plates were made and each was initiated by the addition of 3.3X Start solution containing 5 mM succinate. Plates were incubated in the dark at room temperature for 30-40 minutes before acquiring endpoint fluorescence using a Victor 3V plate reader (Perkin Elmer) equipped with 550/590 nm excitation/emission filters.

The fluorescence values in each well were scaled as the percent change from the intra-plate DMSO controls with the appropriate inhibitor positive controls defined as −100%. Following initial scaling, intra-plate normalized values were subjected to Tukey's two-way median polish to remove row and column dependent positional effects (Malo et al, 2006).

Compounds that demonstrated a reduction in ROS production in this assay include compounds 1-12, 75-77, 84 and 85 as provided in Table 2. Structures corresponding to the compound numbers in Table 2 may be found in Table 1.

TABLE 2
Exemplary compounds that inhibit IQ ROS production.

| Compound | % Reduction in ROS production |
|---|---|
| 1. | −60.6 |
| 2. | −52.5 |
| 3. | −52.4 |
| 4. | −47.9 |
| 5. | −40.0 |
| 6. | −38.6 |
| 7. | −36.5 |
| 8. | −36.3 |
| 9. | −31.2 |
| 10. | −24.1 |
| 11. | −23.8 |
| 12. | −20.7 |
| 75. | −50 |
| 76. | −24 |
| 77. | −18 |
| 84. | −47.7 |
| 85. | −50.6 |

As seen from the table above, a number of selective IQ ROS inhibitors were identified. Compound No. 4, possessed an optimal combination of potency and minimal off-target effects. This compound was assessed in further studies of IQ ROS inhibition. Using 2.5 uM of Compound No. 4, 65% of ROS production from site IQ was suppressed without discernible effects on any other major sites within mitochondria (see FIG. 2A).

Both the uncoupler FCCP and the CI Q-site inhibitor rotenone are effective, though non-specific, inhibitors of electron leak from site IQ. To test if Compound No. 4 is suppressing site IQ ROS via a mechanism distinct from these compounds, respiration both under phosphorylating (ATP producing) and non-phosphorylating conditions using different substrates were measured. Compound No. 4 did not significantly inhibit or show signs of uncoupling respiration under any conditions except at 80 uM. Importantly, at 2.5 uM Compound No. 4 does not inhibit forward electron flow through CI. Next, we directly compared the effects of Compound No. 4, FCCP, and rotenone on the kinetics of electron transport through CI under identical conditions used to drive ROS production in the absence of other inhibitors. For this, succinate was used to drive electrons in reverse through CI to NADH in the presence of a low amount of glutamate to allow full activation of SDH. The rates of NADH reduction and ROS production were then correlated for each putative inhibitor over a range of effective concentrations. Not surprisingly, both rotenone and FCCP progressively inhibited both activities as they altered electron fluxes within CI and the rest of the transport chain. FCCP suppresses observed ROS to near zero as all redox centers (and NADH) remain oxidized. Rotenone shows a similar progression until complete block of the Q-site at higher concentrations eliminates all IQ ROS production but not that from underlying IF and IIIQo. Importantly, at all concentrations where these two compounds reduce observed ROS, the rate of NADH reduction was also impaired significantly. This contrasts remarkably with Compound No. 4 which suppresses observed ROS at least 45% at 2.5 uM before any sign of altered flux through CI to NADH (See FIG. 2B).

The contribution of site IQ to total ROS production under “normal” circumstances (e.g. when electrons enter CI primarily in the forward direction) remains contentious. Recent work on mitochondria respiring solely on CI substrates has demonstrated that the majority of the ROS produced can be assigned to site IF and IIIQo (Quinlan et al, Free Radical Biology and Medicine, online Aug. 16, 2012). Interestingly, when ATP synthesis is restricted, a significant portion of ROS remained unattributed. A possible explanation for this difference is the leak of electrons from site IQ as both Q-pool reduction and PMF remained high in the absence of energy production. To test if site IQ is indeed active under such conditions, we used Compound No. 4 to non-invasively probe mitochondrial ROS production in this system. Addition of 2.5 uM Compound No. 4 lowered the observed ROS production without any changes to the NADH or b566 reduction states. It is believed that even under extreme conditions in which forward electron flow through CI dominates, this provides evidence that site IQ can be a relevant source of ROS.

### Example 5

### Identification of Inhibitors of the Flavin Binding Site of Complex I

To assess the effects of a compound on ROS production from the flavin binding site of complex I (IF), Start Solution containing 5 mM glutamate, 5 mM malate and 4 μM rotenone was distributed into standard 96-well transfer plates. A Biomek FX workstation was programmed to dispense solutions into black 96-well assay plates. Solutions were dispensed to all assay plates in the following order: ROS Detection solution described in Example 3, Mitochondria solution described in Example 1 to a final concentration of 0.3 mg/ml, and Compound dilutions described in Example 2. Duplicate assay plates were made and each was initiated by the addition of 3.3X Start solution containing 5 mM glutamate, 5 mM malate and 4 μM rotenone. Plates were incubated in the dark at room temperature for 30-40 minutes before acquiring endpoint fluorescence using a Victor 3V plate reader (Perkin Elmer) equipped with 550/590 nm excitation/emission filters.

The fluorescence values in each well were scaled as the percent change from the intra-plate DMSO controls with the appropriate inhibitor positive controls defined as −100%. Following initial scaling, intra-plate normalized values were subjected to Tukey's two-way median polish to remove row and column dependent positional effects (Malo, N. et al. (2006) Statistical practice in high-throughput screening data analysis. Nat. Biotechnol. 24(2): 167-75).

Compounds that demonstrated a reduction in ROS production in this assay include compounds 13-33, 75, 76 and 78-82 as provided in Table 3. Structures corresponding to the compound numbers in Table 3 may be found in Table 1.

TABLE 3
Exemplary compounds that inhibit IF ROS production.

| Compound | % Reduction in ROS production |
|---|---|
| 13. | −39.0 |
| 14. | −37.7 |
| 15. | −27.6 |
| 16. | −26.7 |
| 17. | −26.0 |
| 18. | −25.8 |
| 19. | −25.6 |
| 20. | −24.0 |
| 21. | −23.0 |
| 22. | −22.1 |
| 23. | −21.1 |
| 24. | −19.8 |
| 25. | −19.7 |
| 26. | −19.6 |
| 27. | −19.6 |
| 28. | −19.1 |
| 29. | −18.1 |
| 30. | −36.9 |
| 31. | −7.6 |
| 32. | −28.8 |
| 33. | −3.0 |
| 75. | −83 |
| 76. | −34 |
| 78. | −37 |
| 79. | −17 |
| 80. | −23 |
| 81. | −49 |
| 82. | −34 |

As seen from the table above, our screen identified a number of inhibitors that selectively suppressed IF ROS production by an average of 15% or more. Eight compounds were chosen for retesting; three of which shared structural similarities (another 9 showed some non-specific effects even in initial screen). Several compounds were also chosen because they were structurally similar to one of the other top eight hits. Several continued to show effects on IF ROS upon rescreening against an expanded set of ROS and ΔΨm assays, but also displayed uncoupler-like activity.

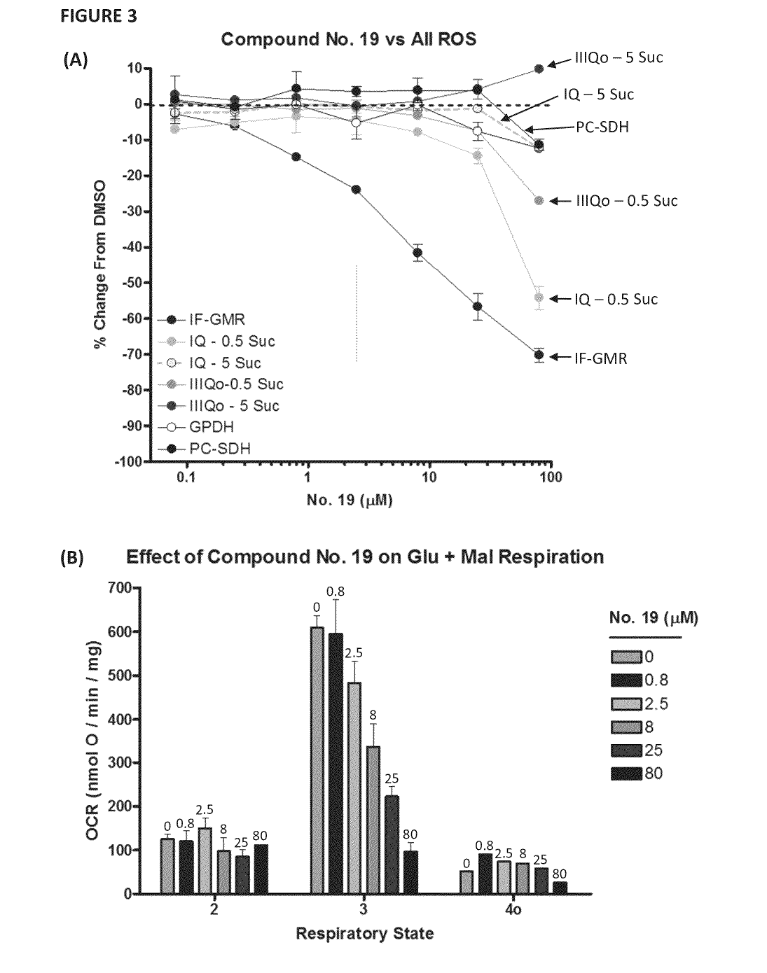

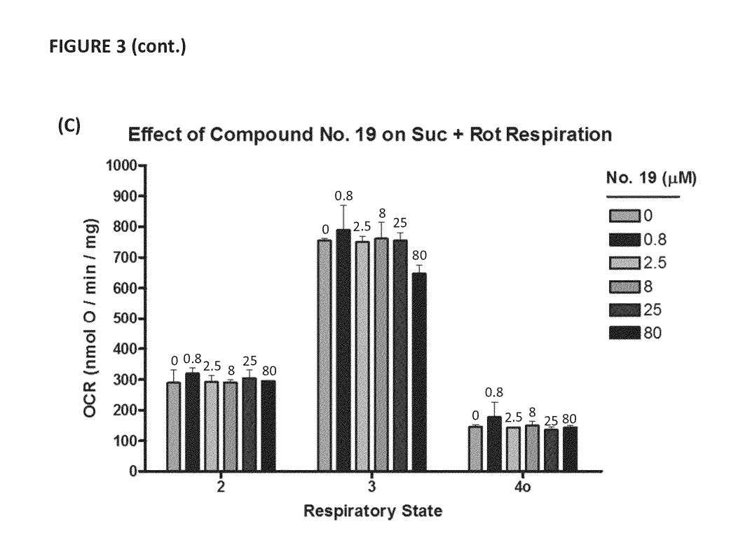

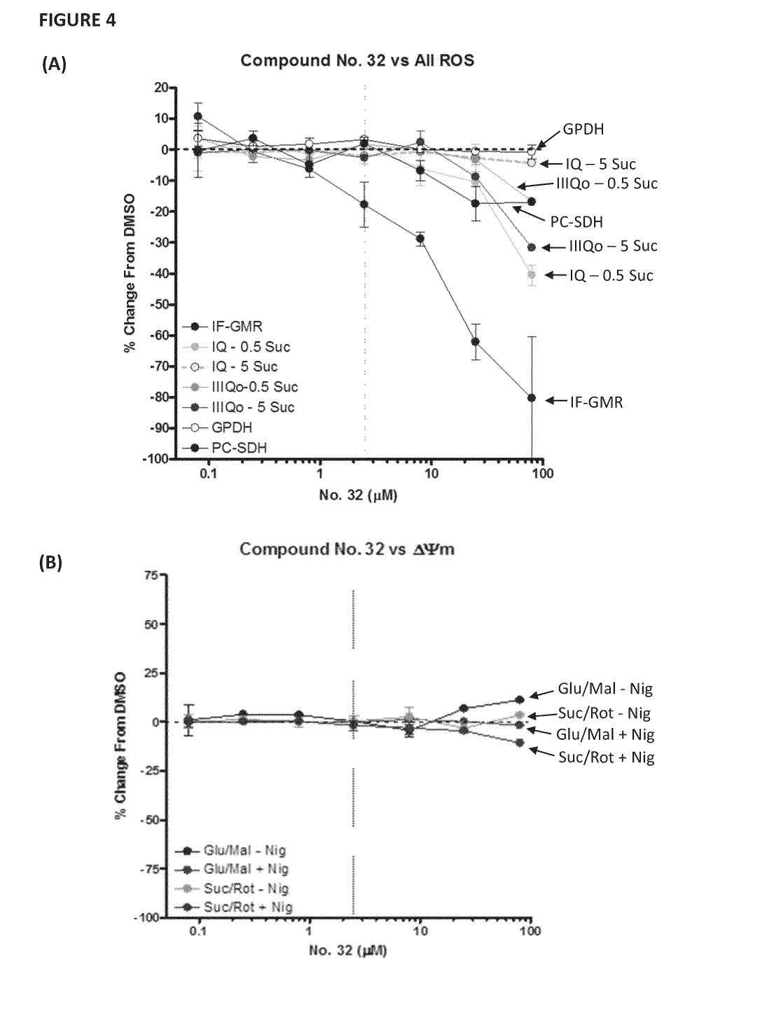

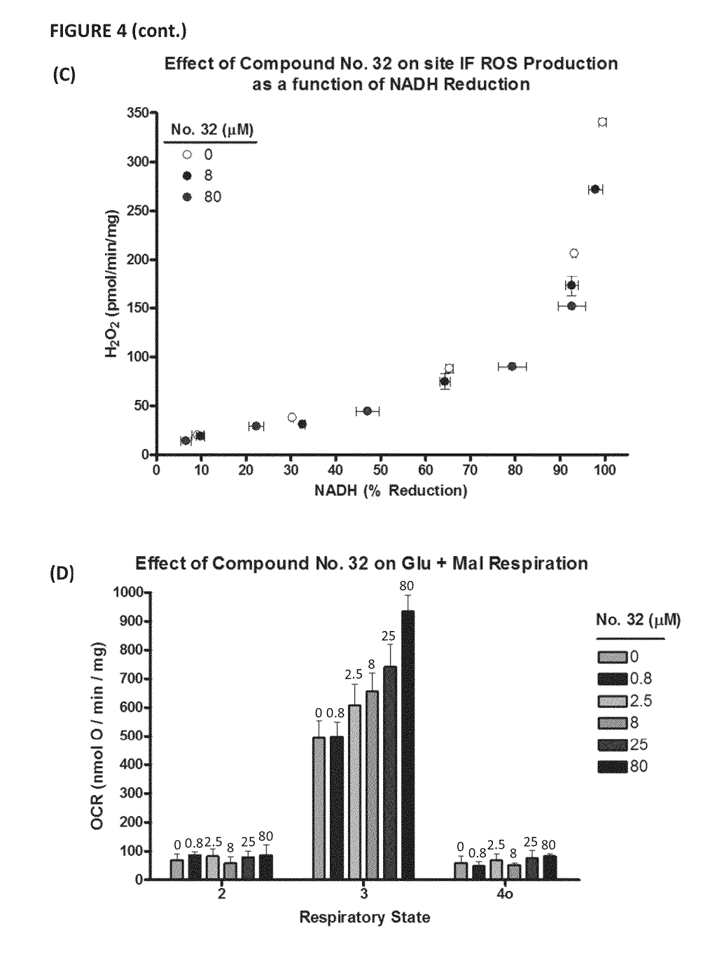

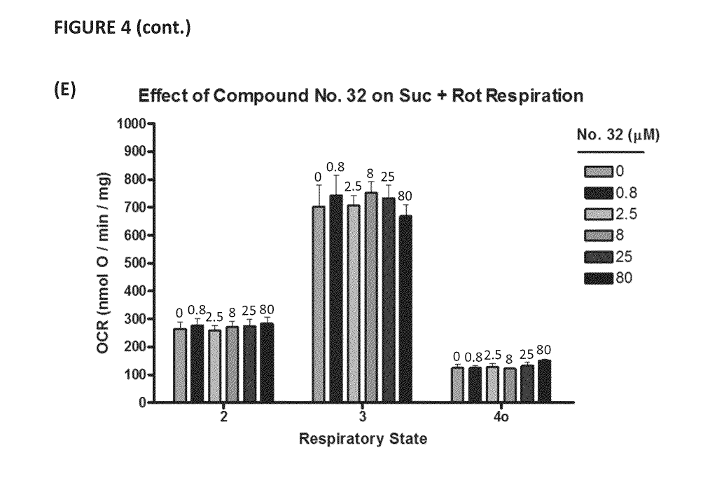

In contrast, two groups of retested IF inhibitors displayed unique properties that set them apart from these weak uncouplers. The first group, exemplified by Compound No. 19 (see FIG. 3) inhibited IF ROS by 24% at 2.5 μM with minimal effects on IQ ROS production driven with subsaturating substrate until 25 μM. IQ ROS production driven with saturating substrate along with IIIQo ROS production was unaffected until the highest concentration tested (80 μM). A structural analog of Compound No. 19 (Compound No. 30), showed increased potency against IF ROS production (37% inhibited at 2.5 μM). Measurement of mitochondrial respiration in the presence of Compound No. 19 indicated no signs of uncoupling but did reveal a progressive inhibition of state 3 respiration (ATP producing) only with CI substrates (glutamate and malate). This class of inhibitors therefore acts independently of Q-site inhibition or uncoupling and appears to target glutamate and malate metabolism upstream of CI. Other analogs of Compound No. 19, such as Compound Nos. 28 and 20 may also be of particular interest.

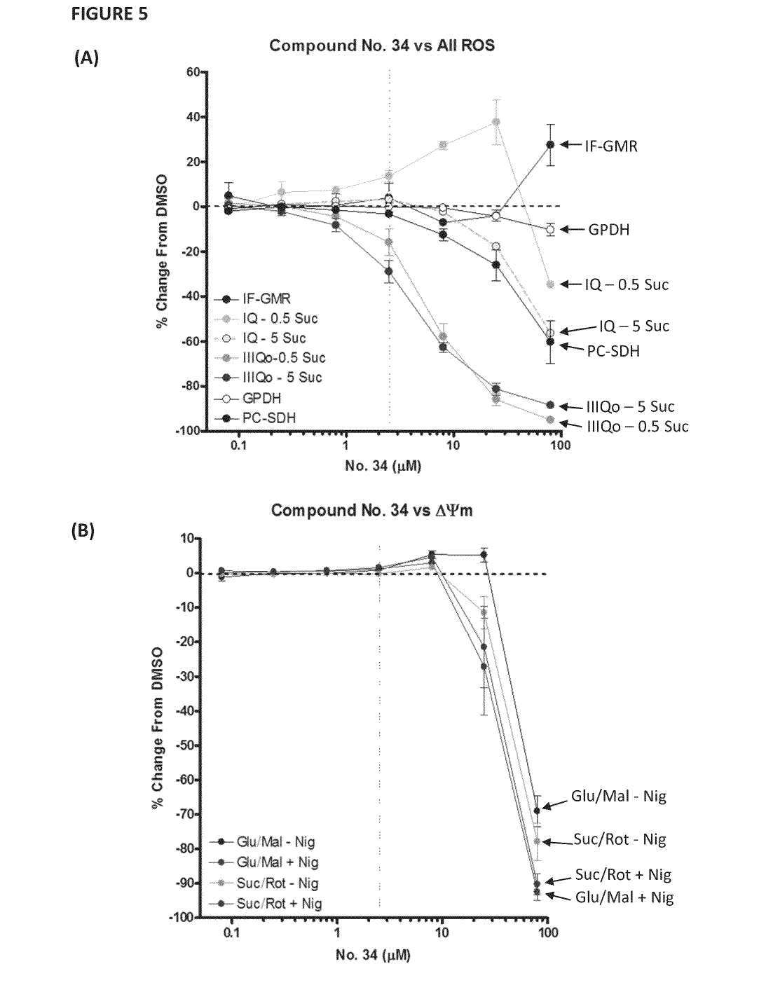

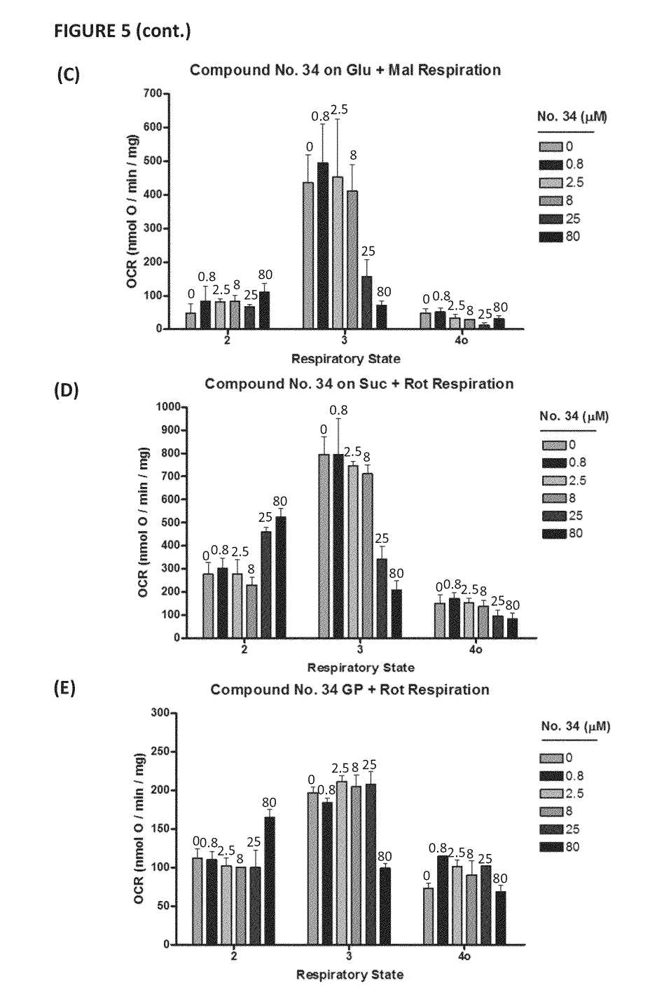

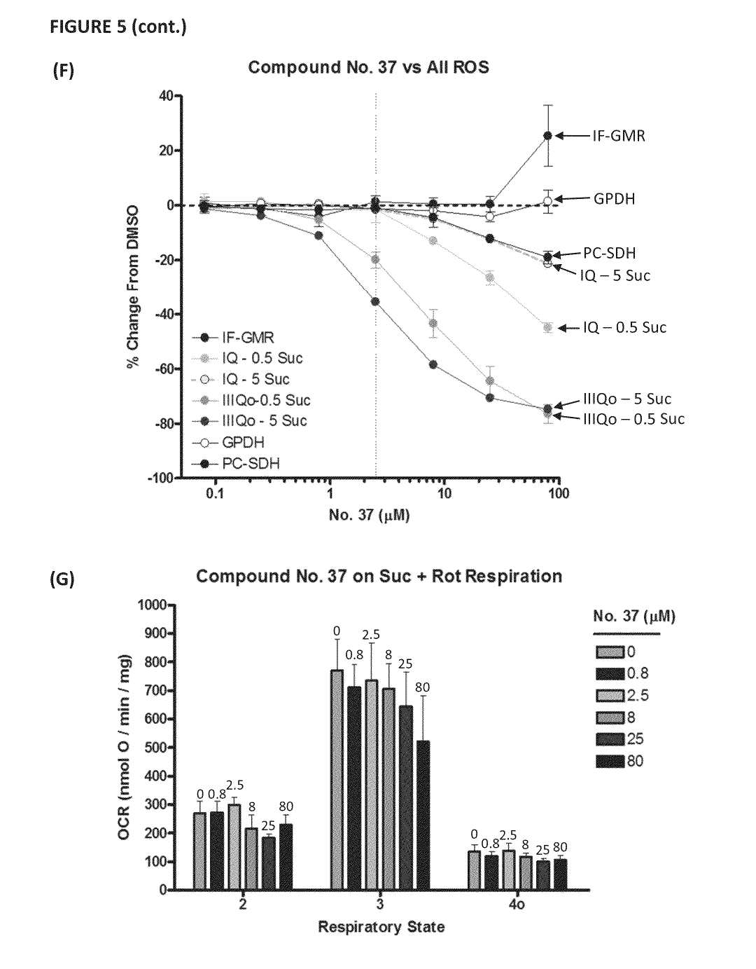

The second group of IF ROS inhibitors was revealed by closer examination of the effects of Compound No. 29. This compound was retested along with three structural analogs, Compound Nos. 33, 32 and 31. Retests of Compound No. 29 showed strong effects on IQ ROS with subsaturating substrate as observed during retesting of other top IF hits. However, the removal of the trifluoromethyl group in Compound No. 31 improved selectivity toward the IF site in combination with a lowered effect on ΔΨm. Additionally, substitution of the trifluormethyl-phenyl with a second N,N-dimethylethylamine arm (Compound Nos. 33 and 32) further improved selectivity for IF without compromising ΔΨm. IF ROS production is proportional to the reduction state of the matrix NADH pool (Treberg et al. (2011). Quinlan refs). Therefore, we measured the ROS:NADH relationship in the presence of varying Compound No. 32. At high NADH reduction (>90%), the expected ROS production was suppressed by increasing amounts of Compound No. 32. Surprisingly, Compound No. 32 also significantly increased state 3 respiration specifically with CI substrates. Therefore, Compound No. 32 is able to significantly suppress ROS production from site IF while facilitating flux through the complex during high demand. See FIG. 5. It is difficult to imagine an indirect mechanism of action to explain both effects on CI activity, so likely acts directly on complex I, but effect on IF ROS is relatively small. Although Compound No. 32 appears to suppress IF ROS only at the high NADH reduction, this situation may be relevant to certain normal and dysfunctional/pathological conditions. Compound Nos. 29 and 31 had stronger off-target effects on IQ ROS when driven by low concentration succinate, suggesting subtle effects on succinate uptake or on membrane potential.

### Example 6

### Identification of Inhibitors of the Outer Ubiquinone Binding Site of Complex III (IIIQ_{O})

To assess the effects of a compound on ROS production from the outer ubiquinone binding site of complex III (IIIQ_{O}), Start Solution containing 5 mM succinate, 4 μM rotenone and 2.5 μM antimycin A was distributed into standard 96-well transfer plates. A Biomek FX workstation was programmed to dispense solutions into black 96-well assay plates. Solutions were dispensed to all assay plates in the following order: ROS Detection solution described in Example 3, Mitochondria solution described in Example 1 to a final concentration of 0.1 mg/ml, and Compound dilutions described in Example 2. Duplicate assay plates were made and each was initiated by the addition of 3.3X Start solution containing 5 mM succinate, 4 μM rotenone and 2.5 μM antimycin A. Plates were incubated in the dark at room temperature for 30-40 minutes before acquiring endpoint fluorescence using a Victor 3V plate reader (Perkin Elmer) equipped with 550/590 nm excitation/emission filters.

The fluorescence values in each well were scaled as the percent change from the intra-plate DMSO controls with the appropriate inhibitor positive controls defined as −100%. Following initial scaling, intra-plate normalized values were subjected to Tukey's two-way median polish to remove row and column dependent positional effects (Malo et al., 2006).

Compounds that demonstrated a reduction in ROS production in this assay include compounds 34-45, 77-80 and 83 as provided in Table 4. Structures corresponding to the compound numbers in Table 4 may be found in Table 1.

TABLE 4
Exemplary compounds that inhibit IIIQ_{O} ROS production.

| Compound | % Reduction in ROS production |
|---|---|
| 34. | −49.4 |
| 35. | −34.9 |
| 36. | −33.6 |
| 37. | −32.1 |
| 38. | −31.3 |
| 39. | −26.8 |
| 40. | −25.1 |
| 41. | −24.7 |
| 42. | −24.1 |
| 43. | −23.2 |
| 44. | −23.1 |
| 45. | −20.2 |
| 77. | −15 |
| 78. | −30 |
| 79. | −19 |
| 80. | −12 |
| 83. | −29 |

As seen from the table above, our screen yielded a number of compounds that selectively suppressed IIIQo ROS production by an average of 20% or more. Two of the retested compounds were particularly selective for site IIIQo. Compound No. 34 inhibited 60% of IIIQo ROS independently of substrate concentration at 8 μM with only subtle effects at other sites. See FIG. 5. Interestingly, Compound No. 34 consistently increased IQ ROS but only with subsaturating substrate. This is the only compound tested in this condition (of over 150) that displayed such an effect and is likely explained by a subtle increase in the PMF (a strong determinant of IQ ROS production). Indeed, a consistent increase in ΔΨm was observed independent of both substrate and ΔpH at 8 μM. This effect on PMF and IQ ROS is likely independent of the IIIQo selectivity of Compound No. 34 since we assay IIIQo ROS production in the absence of PMF. Importantly, these subtle effects at 8 μM did not alter resting (state 2 and 4) or energy producing (state 3) respiration with any substrate. Above 8 μM, Compound No. 34 showed uncoupling-like behavior (collapsed ΔΨm and increased state 2 respiration on all substrates). In contrast to Compound No. 34, another IIIQo selective hit, Compound No. 37, decreased IQ ROS with low substrate and did not uncouple state 2 respiration. Other sites of ROS production and ΔΨm were similarly, but less strongly, affected by Compound No. 37. See FIG. 5. Therefore, some off-target effects of these IIIQo ROS suppressors appear dissociable.

### Example 7

### Identification of Inhibitors of Succinate Dehydrogenase/Complex II (SDH)

To assess the effects of a compound on ROS production from succinate dehydrogenase/complex II (SDH), Start Solution containing 15 μM palmitoyl carnitine, 2.5 μM antimycin A and 2 μM myxothiazol was distributed into standard 96-well transfer plates. A Biomek FX workstation was programmed to dispense solutions into black 96-well assay plates. Solutions were dispensed to all assay plates in the following order: ROS Detection solution described in Example 3, Mitochondria solution described in Example 1 to a final concentration of 0.3 mg/ml, and Compound dilutions described in Example 2. Duplicate assay plates were made and each was initiated by the addition of 3.3X Start solution containing 15 μM palmitoyl carnitine, 2.5 μM antimycin A and 2 μM myxothiazol. Plates were incubated in the dark at room temperature for 30-40 minutes before acquiring endpoint fluorescence using a Victor 3V plate reader (Perkin Elmer) equipped with 550/590 nm excitation/emission filters.

The fluorescence values in each well were scaled as the percent change from the intra-plate DMSO controls with the appropriate inhibitor positive controls defined as −100%. Following initial scaling, intra-plate normalized values were subjected to Tukey's two-way median polish to remove row and column dependent positional effects (Malo et al., 2006).

Compounds that demonstrated a reduction in ROS production in this assay include compounds 71-75, 82 and 83 as provided in Table 5. Structures corresponding to the compound numbers in Table 5 may be found in Table 1.

TABLE 5
Exemplary compounds that inhibit SDH ROS production.

| Compound | % Reduction in ROS Production |
|---|---|
| 71. | −87.6 |
| 72. | −27.4 |
| 73. | −26.9 |
| 74. | −20.9 |
| 75. | −22 |
| 82. | −35 |
| 83. | −14 |

### Example 8

### Identification of Inhibitors of Glycerol-3-Phosphate Dehydrogenase (GPDH)

To assess the effects of a compound on ROS production from glycerol-3-phosphate dehydrogenase (GPDH), Start Solution containing 25 mM glycerol phosphate, 4 μM rotenone, 2.5 μM antimycin A, 1 mM malonate, 2 μM myxothiazol was distributed into standard 96-well transfer plates. A Biomek FX workstation was programmed to dispense solutions into black 96-well assay plates. Solutions were dispensed to all assay plates in the following order: ROS Detection solution described in Example 3, Mitochondria solution described in Example 1 to a final concentration of 0.2 mg/ml, and Compound dilutions described in Example 2. Duplicate assay plates were made and each was initiated by the addition of 3.3X Start solution containing 25 mM glycerol phosphate, 4 μM rotenone, 2.5 μM antimycin A, 1 mM malonate, 2 μM myxothiazol. Plates were incubated in the dark at room temperature for 30-40 minutes before acquiring endpoint fluorescence using a Victor 3V plate reader (Perkin Elmer) equipped with 550/590 nm excitation/emission filters.

The fluorescence values in each well were scaled as the percent change from the intra-plate DMSO controls. GPDH ROS was arbitrarily scaled to the average fluorescence of the positive controls in parallel IQ ROS assays since no potent inhibitors of GPDH ROS exist. This choice was determined to underestimate the potency of compounds on GPDH-specific ROS but was maintained throughout the screen for consistency in subsequent hit identification. Following initial scaling, intra-plate normalized values were subjected to Tukey's two-way median polish to remove row and column dependent positional effects (Malo et al., 2006).

Compounds that demonstrated a reduction in ROS production in this assay include compounds 46-70, 75, 80 and 81 as provided in Table 6. Structures corresponding to the compound numbers in Table 6 may be found in Table 1.

TABLE 6
Exemplary compounds that inhibit GPDH ROS production.

| Compound | % Reduction in ROS Production |
|---|---|
| 46. | −21.6 |
| 47. | −14.1 |
| 48. | −13.4 |
| 49. | −12.7 |
| 50. | −12.2 |
| 51. | −12.0 |
| 52. | −14.6 |
| 53. | 0.0 |
| 54. | 0.9 |
| 55. | 3.3 |
| 56. | 1.5 |
| 57. | −0.8 |
| 58. | 0.2 |
| 59. | −1.2 |
| 60. | −4.4 |
| 61. | −48.3 |
| 62. | −3.8 |
| 63. | −1.7 |
| 64. | 8.2 |
| 65. | −13.0 |
| 66. | −1.9 |
| 67. | 1.8 |
| 68. | −11.1 |
| 69. | −32.1 |
| 70. | −26.0 |
| 75. | −16 |
| 80. | −11 |
| 81. | −13 |

As seen from the table above, a number of compounds that inhibited GPDH ROS production were discovered during screening of the ChemBridge compound library. Four hits were only marginally selective for GPDH ROS and were not pursued further. Promising compounds and analogs were retested against a panel of 8 ROS assays (targeting 5 distinct sites of ROS production) and 4 membrane potential assays (monitoring ΔΨm powered with different substrates using the dye TMRM).

One hit was a false positive for GPDH ROS in that it did not show any effect on GPDH when retested against our expanded set of microplate assays (Compound No. 52). One hit was a false positive in that it progressively inhibited GPDH ROS (Compound No. 47) but also displayed an off-target effect on site IIIQo ROS under conditions similar to the initial screen. In addition, it progressively inhibited other sites of ROS production at higher concentrations. The final two hits (Compound Nos. 50 and 46) were structurally similar, varying in only one atomic position, and retested well as selective inhibitors of GPDH ROS. Compound No. 46 was more potent and selective in both the initial screen and retesting and was selected for further testing in cuvette-based fluorimeter assays where it showed improved selectivity and potency for GPDH ROS.

Further testing of the Compound No. 46 for effects on ΔΨm when driven by glycerol phosphate in either the microplate format or cuvette-based fluorimeter assays indicated it was selective for GPDH (it did not alter ΔΨm driven by complex I or complex II substrates) but also clearly indicated that it was disrupting the enzymatic activity of GPDH by lowering ΔΨm driven by glycerol phosphate under various conditions.

Therefore, we pursued this new class of selective inhibitor of mitochondrial GPDH enzymatic activity even though it did not fit the original criteria for our screen. First, we verified that Compound No. 46 only inhibits mitochondrial respiration or rate of ATP production when glycerol phosphate is substrate. This was confirmed using a Seahorse XF24 analyzer to measure mitochondrial respiration. Next, we verified that Compound No. 46 only inhibits the mitochondrial isoform of GPDH and not the soluble, structurally unrelated, cytosolic isoform of GPDH.

We then analyzed compounds that were structurally similar to Compound Nos. 46 and 50. Importantly, we did find that adding extra rings or elements to the end of the benzimidazole ring increased potency against GPDH activity. However, comparison of those compounds with effects on GPDH activity showed strong correlations between their inhibition of GPDH-specific parameters and off-target effects.

The most potent analog (Compound No. 61) was confirmed to be a more potent inhibitor of GPDH-specific ROS, ΔΨm, and respiration. However, its increased off-target effect on ΔΨm in the absence of nigericin was also confirmed.

Finally, both Compound Nos. 46 and 61 have been tested to determine effects on the kinetics of GPDH activity, inhibitor constants, and IC50s. These inhibitors display mixed inhibition kinetics, lowering the Vmax at intermediate concentrations while increasing the Km for glycerol phosphate at the highest concentrations. This analysis confirms that Compound No. 61 is at least 6 times as potent as Compound No. 46 at inhibiting GPDH activity.

### Example 9

### Identification of Site Specific Inhibitors of ROS Production at Sites IQ, IF, IIIQ_{O}, SDH and GPDH

To identify compounds that specifically inhibit ROS production from a single site of ROS production, compounds were tested in each of the assays described in Examples 4-8 and the effects of the compound on ROS species production from each site were compared.

Specific compounds that demonstrated specific inhibition of ROS production from one of the ROS production sites include compounds 1-74 as provided in Table 7. Structures corresponding to the compound numbers in Table 7 may be found in Table 1.

TABLE 7
Exemplary site specific inhibitors of ROS production.

| Compound | IQ | IF | IIIQO | GPDH | SDH |
|---|---|---|---|---|---|
| 1. | −60.6 | 10.0 | 1.0 | 4.1 | −8.2 |
| 2. | −52.5 | −7.0 | 1.9 | −0.1 | 2.6 |
| 3. | −52.4 | 0.5 | 1.0 | −0.1 | −6.8 |
| 4. | −47.9 | 3.1 | −0.3 | 2.8 | 0.8 |
| 5. | −40.0 | 0.2 | −3.6 | 0.7 | 2.3 |
| 6. | −38.6 | 3.6 | −3.4 | −2.3 | −1.1 |
| 7. | −36.5 | 1.3 | 9.2 | 5.6 | 2.1 |
| 8. | −36.3 | −4.2 | 7.6 | −1.8 | 0.4 |
| 9. | −31.2 | 0.4 | −2.7 | 1.0 | 0.3 |
| 10. | −24.1 | 3.3 | −2.8 | −4.3 | 1.0 |
| 11. | −23.8 | 3.3 | −1.9 | −5.0 | −0.2 |
| 12. | −20.7 | −1.7 | 0.2 | 0.5 | −0.1 |
| 13. | 0.1 | −39.0 | −9.0 | −1.2 | −2.8 |
| 14. | −4.7 | −37.7 | −2.9 | −3.9 | −9.6 |
| 15. | −0.1 | −27.6 | −7.5 | −2.5 | −2.7 |
| 16. | −5.4 | −26.7 | 6.6 | −0.6 | 4.2 |
| 17. | −7.4 | −26.0 | −6.0 | −3.6 | −3.4 |
| 18. | −1.0 | −25.8 | 5.4 | −0.6 | −0.3 |
| 19. | 0.0 | −25.6 | 1.2 | −1.0 | n.d. |
| 20. | 0.2 | −24.0 | 1.2 | −1.7 | −0.7 |
| 21. | 0.0 | −23.0 | −9.9 | −1.8 | 1.7 |
| 22. | −0.4 | −22.1 | −6.2 | −2.6 | 5.5 |
| 23. | −2.7 | −21.1 | −8.8 | −1.0 | 1.7 |
| 24. | −1.0 | −19.8 | −1.3 | −2.0 | −1.5 |
| 25. | −0.6 | −19.7 | −3.3 | −2.5 | −0.1 |
| 26. | −0.8 | −19.6 | −1.8 | −3.3 | n.d. |
| 27. | 0.0 | −19.6 | −0.1 | −0.4 | 0.8 |
| 28. | 0.2 | −19.1 | −2.5 | −2.3 | 1.3 |
| 29. | −2.1 | −18.1 | −3.2 | −2.4 | −0.1 |
| 30. | −0.7 | −36.9 | 0.1 | 0.7 | 3.3 |
| 31. | −1.4 | −7.6 | −1.4 | 0.5 | −6.0 |
| 32. | −0.6 | −28.8 | 2.5 | 0.2 | −6.8 |
| 33. | −0.5 | −3.0 | 6.1 | −12.7 | n.d. |
| 34. | 4.0 | 5.6 | −49.4 | −4.0 | 3.9 |
| 35. | 1.3 | −0.7 | −34.9 | −5.3 | 0.2 |
| 36. | 3.7 | 0.6 | −33.6 | −4.9 | −1.1 |
| 37. | 0.3 | 0.6 | −32.1 | −0.6 | 1.2 |
| 38. | −3.9 | −1.8 | −31.3 | 3.7 | n.d. |
| 39. | 2.7 | −0.9 | −26.8 | −4.7 | n.d. |
| 40. | 4.3 | 1.8 | −25.1 | −4.2 | −2.7 |
| 41. | 0.2 | 6.6 | −24.7 | 2.6 | 1.2 |
| 42. | 7.9 | −2.6 | −24.1 | −0.3 | 0.7 |
| 43. | −0.5 | −4.4 | −23.2 | 3.4 | n.d. |
| 44. | 0.4 | 3.1 | −23.1 | −3.4 | n.d. |
| 45. | −0.2 | 0.5 | −20.2 | 3.1 | n.d. |
| 46. | 1.0 | 4.1 | −0.2 | −21.6 | 3.6 |
| 47. | −0.1 | −0.5 | 0.0 | −14.1 | 1.3 |
| 48. | −2.9 | −6.5 | −5.7 | −13.4 | n.d. |
| 49. | 1.7 | 1.0 | 10.0 | −12.7 | −1.0 |
| 50. | 0.8 | −2.6 | 2.5 | −12.2 | 2.1 |
| 51. | −2.8 | 5.5 | −9.8 | −12.0 | −1.8 |
| 52. | −0.2 | −2.2 | −0.4 | 0.0 | 4.6 |
| 53. | 0.1 | 1.3 | 0.8 | 0.9 | −0.3 |
| 54. | 2.3 | 8.0 | 2.5 | 3.3 | 4.4 |
| 55. | −3.9 | −1.9 | 3.3 | 1.5 | 1.5 |
| 56. | −0.3 | 0.3 | 1.4 | −0.8 | −2.9 |
| 57. | 3.1 | 1.7 | 0.4 | 0.2 | 4.2 |
| 58. | −6.4 | 0.1 | 0.8 | −1.2 | 1.5 |
| 59. | −12.3 | −3.0 | −1.3 | −4.4 | −5.1 |
| 60. | −43.9 | −4.2 | 1.1 | −48.3 | −3.4 |
| 61. | −17.1 | −4.8 | −2.4 | −3.8 | −4.5 |
| 62. | −11.7 | −3.4 | −1.1 | −1.7 | −3.1 |
| 63. | −6.9 | 0.9 | 1.8 | 8.2 | 3.5 |
| 64. | −15.3 | −3.8 | −1.0 | −13.0 | 9.8 |
| 65. | −11.0 | −1.3 | −1.0 | −1.9 | 0.2 |
| 66. | −2.8 | −0.2 | −1.2 | 1.8 | −2.5 |
| 67. | −11.7 | −10.1 | −6.5 | −11.1 | −16.8 |
| 68. | −23.0 | −1.3 | 1.1 | −32.1 | −3.7 |
| 69. | −20.1 | −1.8 | 5.5 | −26.0 | −6.3 |
| 70. | −0.2 | −2.2 | −0.4 | 0.0 | 4.6 |
| 71. | 1.6 | 7.0 | 6.3 | −0.6 | −87.6 |
| 72. | 0.4 | 2.6 | 5.4 | 1.7 | −27.4 |
| 73. | −4.6 | −0.7 | −1.3 | −2.4 | −26.9 |
| 74. | −0.7 | 0.4 | −7.6 | −3.5 | −20.9 |
| 84. | −47.7 | −1.4 | 0.8 | −0.2 | 0.7 |
| 85. | −50.6 | 0.5 | −0.8 | −3.3 | 3.6 |

### Example 10

### Determination of the Effects of Compounds on Membrane Potential (ΔΨm)

Compounds were manually transferred from the Master Compound Plate to two black 96-well assay plates. A bulk solution was prepared containing the following components (final concentrations) and manually dispensed into the two ΔΨm assay plates: the potentiometric dye TMRM (5 μM), mitochondria (0.2 mg/mL), glutamate (5 mM), malate (5 mM), and nigericin (80 ng/mL). Nigericin exchanges potassium ions for protons across the mitochondrial inner membrane and collapses ΔpH while maximizing the electrical component (ΔΨm) of the proton motive force. Pilot screening indicated that nigericin made the ΔΨm assay more robust, less variable, and less ambiguous for revealing unwanted compounds that disrupt ΔΨm by inhibiting electron transport or uncoupling the proton motive force. ΔΨm assay plates were incubated in the dark at room temperature for 10-15 minutes before acquiring endpoint fluorescence using a Victor 3V plate reader (Perkin Elmer) equipped with 550/590 nm excitation/emission filters.

### Example 11

### Identification of Compounds that Decrease ROS Production while not Affecting Membrane Potential

To identify compounds that inhibit ROS production from one or more sites of ROS production while having minimal effects on ΔΨm, compounds were tested in each of the assays described in Examples 4-8 and 10 and the effects of the compound on ROS species production from each site and ΔΨm were compared.

Specific compounds that inhibit ROS production from at least one of the ROS production sites IQ, IF, IIIQO, SDH and GPDH, while having minimal effects on ΔΨm included compounds 1-51 and 71-83 as provided in Table 8. Structures corresponding to the compound numbers in Table 8 may be found in Table 1.

TABLE 8
Exemplary inhibitors of ROS production that do not affect ΔΨm.

| Compound | IQ | IF | IIIQO | GPDH | SDH | ΔΨm |
|---|---|---|---|---|---|---|
| 1. | −60.6 | 10.0 | 1.0 | 4.1 | −8.2 | −1.5 |
| 2 | −52.5 | −7.0 | 1.9 | −0.1 | 2.6 | −3.4 |
| 3. | −52.4 | 0.5 | 1.0 | −0.1 | −6.8 | 1.2 |
| 4. | −47.9 | 3.1 | −0.3 | 2.8 | 0.8 | −0.9 |
| 5. | −40.0 | 0.2 | −3.6 | 0.7 | 2.3 | −3.5 |
| 6. | −38.6 | 3.6 | −3.4 | −2.3 | −1.1 | 0.0 |
| 7. | −36.5 | 1.3 | 9.2 | 5.6 | 2.1 | −0.5 |
| 8. | −36.3 | −4.2 | 7.6 | −1.8 | 0.4 | −0.3 |
| 9. | −31.2 | 0.4 | −2.7 | 1.0 | 0.3 | −0.1 |
| 10. | −24.1 | 3.3 | −2.8 | −4.3 | 1.0 | −0.2 |
| 11. | −23.8 | 3.3 | −1.9 | −5.0 | −0.2 | −0.4 |
| 12. | −20.7 | −1.7 | 0.2 | 0.5 | −0.1 | −0.5 |
| 13. | 0.1 | −39.0 | −9.0 | −1.2 | −2.8 | −0.5 |
| 14. | −4.7 | −37.7 | −2.9 | −3.9 | −9.6 | −0.7 |
| 15. | −0.1 | −27.6 | −7.5 | −2.5 | −2.7 | −0.9 |
| 16. | −5.4 | −26.7 | 6.6 | −0.6 | 4.2 | −0.3 |
| 17. | −7.4 | −26.0 | −6.0 | −3.6 | −3.4 | 0.3 |
| 18. | −1.0 | −25.8 | 5.4 | −0.6 | −0.3 | −0.7 |
| 19. | 0.0 | −25.6 | 1.2 | −1.0 | n.d. | −1.4 |
| 20. | 0.2 | −24.0 | 1.2 | −1.7 | −0.7 | −0.3 |
| 21. | 0.0 | −23.0 | −9.9 | −1.8 | 1.7 | −0.3 |
| 22. | −0.4 | −22.1 | −6.2 | −2.6 | 5.5 | −0.9 |
| 23. | −2.7 | −21.1 | −8.8 | −1.0 | 1.7 | −1.5 |
| 24. | −1.0 | −19.8 | −1.3 | −2.0 | −1.5 | 0.1 |
| 25. | −0.6 | −19.7 | −3.3 | −2.5 | −0.1 | −0.4 |
| 26. | −0.8 | −19.6 | −1.8 | −3.3 | n.d. | −0.7 |
| 27. | 0.0 | −19.6 | −0.1 | −0.4 | 0.8 | 1.6 |
| 28. | 0.2 | −19.1 | −2.5 | −2.3 | 1.3 | −1.3 |
| 29. | −2.1 | −18.1 | −3.2 | −2.4 | −0.1 | −0.6 |
| 30. | −0.7 | −36.9 | 0.1 | 0.7 | 3.3 | −1.0 |
| 31. | −1.4 | −7.6 | −1.4 | 0.5 | −6.0 | 0.3 |
| 32. | −0.6 | −28.8 | 2.5 | 0.2 | −6.8 | −0.2 |
| 33. | −0.5 | −3.0 | 6.1 | −12.7 | n.d. | 0.4 |
| 34. | 4.0 | 5.6 | −49.4 | −4.0 | 3.9 | 3.4 |
| 35. | 1.3 | −0.7 | −34.9 | −5.3 | 0.2 | 0.6 |
| 36. | 3.7 | 0.6 | −33.6 | −4.9 | −1.1 | 0.0 |
| 37. | 0.3 | 0.6 | −32.1 | −0.6 | 1.2 | 1.5 |
| 38. | −3.9 | −1.8 | −31.3 | 3.7 | n.d. | 0.3 |
| 39. | 2.7 | −0.9 | −26.8 | −4.7 | n.d. | 0.5 |
| 40. | 4.3 | 1.8 | −25.1 | −4.2 | −2.7 | 0.7 |
| 41. | 0.2 | 6.6 | −24.7 | 2.6 | 1.2 | 2.3 |
| 42. | 7.9 | −2.6 | −24.1 | −0.3 | 0.7 | 0.4 |
| 43. | −0.5 | −4.4 | −23.2 | 3.4 | n.d. | 0.6 |
| 44. | 0.4 | 3.1 | −23.1 | −3.4 | n.d. | 1.0 |
| 45. | −0.2 | 0.5 | −20.2 | 3.1 | n.d. | 0.3 |
| 46. | 1.0 | 4.1 | −0.2 | −21.6 | 3.6 | 0.2 |
| 47. | −0.1 | −0.5 | 0.0 | −14.1 | 1.3 | 0.0 |
| 48. | −2.9 | −6.5 | −5.7 | −13.4 | n.d. | 0.0 |
| 49. | 1.7 | 1.0 | 10.0 | −12.7 | −1.0 | −0.3 |
| 50. | 0.8 | −2.6 | 2.5 | −12.2 | 2.1 | 0.8 |
| 51. | −2.8 | 5.5 | −9.8 | −12.0 | −1.8 | 0.9 |
| 71. | 1.6 | 7.0 | 6.3 | −0.6 | −87.6 | n.d. |
| 72. | 0.4 | 2.6 | 5.4 | 1.7 | −27.4 | 1.0 |
| 73. | −4.6 | −0.7 | −1.3 | −2.4 | −26.9 | −0.2 |
| 74. | −0.7 | 0.4 | −7.6 | −3.5 | −20.9 | 0.8 |
| 75. | −50 | −83 | 3 | −16 | −22 | −2 |
| 76. | −24 | −34 | 0 | 0 | 3 | 0 |
| 77. | −18 | −6 | −15 | 1 | 0 | 1 |
| 78. | 2 | −37 | −30 | −6 | −5 | −1 |
| 79. | 0 | −17 | −19 | −1 | nd | 1 |
| 80. | 0 | −23 | −12 | −11 | 4 | 0 |
| 81. | 2 | −49 | 0 | −13 | −1 | −1 |
| 82. | −2 | −34 | −10 | 0 | −35 | 0 |
| 83. | 2 | −1 | −29 | −3 | −14 | 1 |
| 84. | −47.7 | −1.4 | 0.8 | −0.2 | 0.7 | −1.5 |
| 85. | −50.6 | 0.5 | −0.8 | −3.3 | 3.6 | −0.9 |

### Example 12

### Identification of Compounds that Specifically Decrease ROS Production from a Single ROS Production Site (IQ, IF, IIIQ_{O}, SDH and GPDH) while not Affecting Membrane Potential

To identify compounds that inhibit ROS production from one of the ROS production sites IQ, IF, IIIQ_{O}, SDH and GPDH while having minimal effects on ΔΨm, compounds were tested in each of the assays described in Examples 4-8 and 10 and the effects of the compound on ROS species production from each site and ΔΨm were compared.

Specific compounds that demonstrated specific inhibition of ROS production from one of the ROS production sites IQ, IF, IIIQO, SDH or GPDH, while having minimal effects on ROS production at the remaining ROS production sites IQ, IF, IIIQO, SDH and GPDH and having minimal effects on ΔΨm included compounds 1-51 and 71-74 as provided in Table 9. Structures corresponding to the compound numbers in Table 9 may be found in Table 1.

TABLE 9
Exemplary site specific inhibitors of ROS production
that do not inhibit ΔΨm.

| Compound | IQ | IF | IIIQo | GPDH | SDH | ΔΨm |
|---|---|---|---|---|---|---|
| 1. | −60.6 | 10.0 | 1.0 | 4.1 | −8.2 | −1.5 |
| 2. | −52.5 | −7.0 | 1.9 | −0.1 | 2.6 | −3.4 |
| 3. | −52.4 | 0.5 | 1.0 | −0.1 | −6.8 | 1.2 |
| 4. | −47.9 | 3.1 | −0.3 | 2.8 | 0.8 | −0.9 |
| 5. | −40.0 | 0.2 | −3.6 | 0.7 | 2.3 | −3.5 |
| 6. | −38.6 | 3.6 | −3.4 | −2.3 | −1.1 | 0.0 |
| 7. | −36.5 | 1.3 | 9.2 | 5.6 | 2.1 | −0.5 |
| 8. | −36.3 | −4.2 | 7.6 | −1.8 | 0.4 | −0.3 |
| 9. | −31.2 | 0.4 | −2.7 | 1.0 | 0.3 | −0.1 |
| 10. | −24.1 | 3.3 | −2.8 | −4.3 | 1.0 | −0.2 |
| 11. | −23.8 | 3.3 | −1.9 | −5.0 | −0.2 | −0.4 |
| 12. | −20.7 | −1.7 | 0.2 | 0.5 | −0.1 | −0.5 |
| 13. | 0.1 | −39.0 | −9.0 | −1.2 | −2.8 | −0.5 |
| 14. | −4.7 | −37.7 | −2.9 | −3.9 | −9.6 | −0.7 |
| 15. | −0.1 | −27.6 | −7.5 | −2.5 | −2.7 | −0.9 |
| 16. | −5.4 | −26.7 | 6.6 | −0.6 | 4.2 | −0.3 |
| 17. | −7.4 | −26.0 | −6.0 | −3.6 | −3.4 | 0.3 |
| 18. | −1.0 | −25.8 | 5.4 | −0.6 | −0.3 | −0.7 |
| 19. | 0.0 | −25.6 | 1.2 | −1.0 | n.d. | −1.4 |
| 20. | 0.2 | −24.0 | 1.2 | −1.7 | −0.7 | −0.3 |
| 21. | 0.0 | −23.0 | −9.9 | −1.8 | 1.7 | −0.3 |
| 22. | −0.4 | −22.1 | −6.2 | −2.6 | 5.5 | −0.9 |
| 23. | −2.7 | −21.1 | −8.8 | −1.0 | 1.7 | −1.5 |
| 24. | −1.0 | −19.8 | −1.3 | −2.0 | −1.5 | 0.1 |
| 25. | −0.6 | −19.7 | −3.3 | −2.5 | −0.1 | −0.4 |
| 26. | −0.8 | −19.6 | −1.8 | −3.3 | n.d. | −0.7 |
| 27. | 0.0 | −19.6 | −0.1 | −0.4 | 0.8 | 1.6 |
| 28. | 0.2 | −19.1 | −2.5 | −2.3 | 1.3 | −1.3 |
| 29. | −2.1 | −18.1 | −3.2 | −2.4 | −0.1 | −0.6 |
| 30. | −0.7 | −36.9 | 0.1 | 0.7 | 3.3 | −1.0 |
| 31. | −1.4 | −7.6 | −1.4 | 0.5 | −6.0 | 0.3 |
| 32. | −0.6 | −28.8 | 2.5 | 0.2 | −6.8 | −0.2 |
| 33. | −0.5 | −3.0 | 6.1 | −12.7 | n.d. | 0.4 |
| 34. | 4.0 | 5.6 | −49.4 | −4.0 | 3.9 | 3.4 |
| 35. | 1.3 | −0.7 | −34.9 | −5.3 | 0.2 | 0.6 |
| 36. | 3.7 | 0.6 | −33.6 | −4.9 | −1.1 | 0.0 |
| 37. | 0.3 | 0.6 | −32.1 | −0.6 | 1.2 | 1.5 |
| 38. | −3.9 | −1.8 | −31.3 | 3.7 | n.d. | 0.3 |
| 39. | 2.7 | −0.9 | −26.8 | −4.7 | n.d. | 0.5 |
| 40. | 4.3 | 1.8 | −25.1 | −4.2 | −2.7 | 0.7 |
| 41. | 0.2 | 6.6 | −24.7 | 2.6 | 1.2 | 2.3 |
| 42. | 7.9 | −2.6 | −24.1 | −0.3 | 0.7 | 0.4 |
| 43. | −0.5 | −4.4 | −23.2 | 3.4 | n.d. | 0.6 |
| 44. | 0.4 | 3.1 | −23.1 | −3.4 | n.d. | 1.0 |
| 45. | −0.2 | 0.5 | −20.2 | 3.1 | n.d. | 0.3 |
| 46. | 1.0 | 4.1 | −0.2 | −21.6 | 3.6 | 0.2 |
| 47. | −0.1 | −0.5 | 0.0 | −14.1 | 1.3 | 0.0 |
| 48. | −2.9 | −6.5 | −5.7 | −13.4 | n.d. | 0.0 |
| 49. | 1.7 | 1.0 | 10.0 | −12.7 | −1.0 | −0.3 |
| 50. | 0.8 | −2.6 | 2.5 | −12.2 | 2.1 | 0.8 |
| 51. | −2.8 | 5.5 | −9.8 | −12.0 | −1.8 | 0.9 |
| 71. | 1.6 | 7.0 | 6.3 | −0.6 | −87.6 | n.d. |
| 72. | 0.4 | 2.6 | 5.4 | 1.7 | −27.4 | 1.0 |
| 73. | −4.6 | −0.7 | −1.3 | −2.4 | −26.9 | −0.2 |
| 74. | −0.7 | 0.4 | −7.6 | −3.5 | −20.9 | 0.8 |
| 84. | −47.7 | −1.4 | 0.8 | −0.2 | 0.7 | −1.5 |
| 85. | −50.6 | 0.5 | −0.8 | −3.3 | 3.6 | −0.9 |

### Example 13

### Retesting Compounds Against an Expanded Panel of ROS and ΔΨm Assays

Master Titration Plates (Common to all Retest Assays):

Compounds identified as site-selective hits as well as structural analogs were re-sourced from ChemBridge and dissolved in DMSO as 80 mM stocks. Master Titration Plates were prepared manually by serially diluting 80 mM compound stocks between 0.08 and 80 mM on 96-well microplates. Four compounds were titrated in duplicate on each plate. Additional DMSO controls were interspersed across these plates to serve as local normalization controls during analysis since two-way median polishing was not appropriate. Just prior to use, compounds were diluted 100-fold with standard buffer (10X). The same known inhibitor positive controls were included on each plate as during high-throughput screening.

ROS Detection Solution (Common to all Retest Assays):

Just prior to use, a bulk 3.3X solution was made in standard buffer and distributed into a 2 mL deep well 96-well transfer plate.

Mitochondria Solution (Common to all Retest Assays):

Just prior to use, mitochondria were diluted in standard buffer (0.67 mg/mL) and distributed into a 2 mL deep well 96-well transfer plate.

ROS Assay and ΔΨm Start Solutions (Unique for Each Retest Assay):

The five core ROS assays and single ΔΨm assay were carried out as in the initial high-throughput screen. In addition, several assays were included to help validate the specificity of the compounds for a given site of production. All compounds were titrated against separate assays targeting IQ ROS and IIIQo ROS using subsaturating (0.5 mM final) succinate. Pilot screening revealed these additional assays to be reliable tests of whether or not a compound was acting at the putative sites of production rather than having off-target effects (e.g. subtle effects on proton motive force or substrate uptake or oxidation). All compounds were also titrated against three additional ΔΨm assays to help identify false negative compounds that escaped detection in the initial screen and to reveal concentrations at which compounds begin to exhibit off-target effects. Specifically, ΔΨm was monitored separately with 5 mM succinate (in the presence of 4 μM rotenone) in addition to glutamate and malate as before. The use of different substrates was designed to reveal compounds that disrupt mitochondrial function in a substrate specific manner. Each ΔΨm assay was also run in both the presence and absence of nigericin to help reveal potential mechanisms of action for compounds that exhibited effects on the proton motive force.

In addition to these changes applied for all retested compounds, several assays were designed and implemented in a more selective manner to validate specificity or reveal mechanisms of action. Most frequently, these changes involved the use of different substrates to supply electrons from an alternative entry point in the chain. If a site of ROS production is being targeted selectively by a compound, then in many cases the supply of electrons should not influence the effectiveness of this compound at suppressing ROS production. Variation from this expected outcome may suggest more subtle off-target effects of a compound. Alternative conditions are listed below:
1) IQ ROS: 5 mM succinate with 1 mM glutamate
2) IF ROS: 5 mM malate only as substrate (in the presence of 4 μM rotenone)
3) IIIQo ROS: 5 mM succinate (in the presence of 3 mM malonate, 4 uM rotenone, and 2.5 μM antimycin A)
4) IIIQo ROS: 25 mM glycerol phosphate (in the presence of 1 mM malonate, 4 μM rotenone, and 2.5 μM antimycin A)
5) SDH: 0.5 mM succinate (in the presence of 4 uM rotenone, 2.5 uM antimycin A, and 2 μM myxothiazol)
6) GPDH: Vary the concentration of glycerol phosphate in the presence or absence of 250 nM free calcium to allosterically activate GPDH (all in the presence of 4 μM rotenone, 2.5 uM antimycin A, 1 mM malonate, and 2 μM myxothiazol)
7) ΔΨm: Vary the concentration of glycerol phosphate in the presence or absence of 250 nM free calcium to allosterically activate GPDH and in the presence or absence of 80 ng/mL nigericin (all in the presence of 4 μM rotenone)

ROS assays were setup on the Biomek FX workstation while ΔΨm assays were setup manually. Plates were incubated in the dark at room temperature for 10-40 minutes before acquiring endpoint fluorescence using a Victor 3V plate reader (Perkin Elmer) equipped with 550/590 nm excitation/emission filters.

Data Analysis:

The fluorescence values in each well were scaled in relation to the intra-plate DMSO and known inhibitor controls as in the high-throughput screen except that median values for local interspersed DMSO wells (3-6 wells) were used rather than just the 8 DMSO wells in columns 1 and 12. This normalization was applied in place of the two-way median polish used during high-throughput screening to minimize row and column effects. Certain retest compounds were identified as those that demonstrated progressive, dose-dependent inhibition of ROS production from a single site over at least a 5-10 fold concentration range without strong effects on other sites of ROS production or the maintenance of ΔΨm. At this stage, strong candidates were not necessarily excluded from further testing if subtle off-target effects were observed (e.g. loss of specificity or signs of general inhibition at the highest concentrations). Rather, these candidates were often pursued for more detailed mechanistic studies with the expectation that subsequent structure activity analysis would provide insight into which structurally related compounds would provide the optimal specificity for a given site of production.

### Example 14

### Retesting Compounds Using Cuvette Based ROS and ΔΨm Assays

Candidate compounds that remained site-selective following robotics assisted retesting were subsequently tested for effects on ROS production and ΔΨm with a Varian Cary Eclipse fluorimeter (excitation 550 nm, emission at 590 nm). This experimental format allows kinetic measurements of ROS production and ΔΨm with more accurate temperature control, sample stirring, and background correction. In addition, rates of ROS production can be calibrated to H₂O₂ standards under identical conditions while auto-fluorescence and fluorescence quenching by candidate compounds can be more accurately evaluated. Conditions were generally as described for the various microplate assays with the following exceptions: HRP was increased to 5 units/mL, Amplex UltraRed was increased to 50 μM, temperature was always 37° C., samples were continuously stirred, and baseline rates of fluorescence change in the absence of substrate were acquired prior to their addition. Example protocols for cuvette based measurement of site IQ and site IIIQo ROS production are described in Affourtit et al., 2012. Example methods for cuvette based measurement of ΔΨm (using a fluorescent potentiometric dye analogous to TMRM) are described in Lambert, A. et al. (2008) Diphenyleneiodonium acutely inhibits reactive oxygen species production by mitochondrial complex I during reverse, but not forward electron transport. Biochim. Biophys. Acta. 1777(5): 397-403. An additional advantage of the kinetic cuvette based assays was the ability to perform inline additions to monitor the relative effect of a test compound against multiple conditions in the same cuvette. Therefore, the selectivity of a compound for one site of ROS production can be monitored by adding substrates and select known inhibitors in defined sequences to alternately provide and trap electrons at various sites in the chain. Similarly, ΔΨm can be maintained by substrates via complex I (glutamate and malate), complex II (succinate), or GPDH (glycerol phosphate) through the sequential addition of substrates and selective inhibitors.

### Example 15

### Measurement of Respiration with Seahorse XF Technology

Measurements of ΔΨm described in the previous examples were made under “resting” conditions in which substrates were present but ΔΨm was not coupled to ATP (energy) production by mitochondria. To test the effect of candidate compounds on mitochondrial bioenergetics under energy producing conditions, respiratory coupling parameters were measured in isolated mitochondria in microplate format using a Seahorse XF 24 analyzer (Rogers, G. et al. (2011) High Throughput Microplate Respiratory Measurements Using Minimal Quantities of Isolated Mitochondria. PLoS One. 6(7): e21746). Briefly, plate-attached mitochondria (2-4 μg/well) were exposed to test compounds between 0.8-80 μM in the presence of complex I (glutamate and malate), complex II (succinate with rotenone), or GPDH (glycerol phosphate with rotenone) substrates to determine basal respiration rates (state 2 respiration). Subsequently, ADP (5 mM) was injected and the rate of respiration coupled to ATP production (state 3) was measured. ATP synthesis was terminated by injection of oligomycin (0.5 μg/mL) and leak-dependent respiration was measured (state 4o). Finally, the complex III inhibitors antimycin A (2.5 uM) and myxothiazol (2 μM) were injected to terminate respiration. Respiratory profiles were evaluated for signs of inhibition (decreased states 2, 3, or 4o) or uncoupling (increased states 2 or 4o) by candidate compounds. Seahorse XF experiments were performed in a mannitol-sucrose based buffer as described in Rogers et al. 2011. For respiration experiments driven by glycerol phosphate, this buffer was supplemented with CaCl₂ to a final free concentration of 250 nM.

### Example 16

### Measurement of Steady State Matrix NAD(P)H Reduction

The rate of ROS production from site IF is linked to the reduction state of the NAD(P)H pool in the mitochondrial matrix (Treberg et al., 2011; Quinlan et al, submitted). Therefore, to evaluate the mechanism of action of putative IF ROS inhibitors, the effect of candidate compounds on the relationship between site IF ROS production and NAD(P)H reduction was tested. NAD(P)H reduction was monitored in isolated mitochondria (0.3 mg/mL) on a Varian Cary Eclipse fluorimeter (ex 365/em 450 nm) as described by Treberg et al., 2011 except the reduction state was slowly titrated with increasing rotenone (0.08 to 4 uM) in the presence of saturating malate (5 mM) and the uncoupler FCCP. Glutamate (5 mM) was added to achieve 100% reduction. Candidate IF ROS inhibitors were expected to lower ROS production without significantly altering the relative reduction state of the NAD(P)H pool. Additionally, the reduction state of matrix NAD(P)H can be measured in the presence of candidate compounds that target sites other than site IF as further validation that these compounds do not exert their effects through subtle changes at site IF (or the oxidation of substrates linked to NADH reduction).

### Example 17

### Measurement of the Rate of NAD(P)H Reduction

To compare candidate IQ ROS inhibitors with mitochondrial inhibitors known to disrupt complex I activity (e.g. rotenone) or oxidize the electron transport chain (e.g. the uncoupler FCCP), the rate of NAD(P)H reduction via complex I was measured when isolated mitochondria were provided with saturating concentrations of the complex II substrate succinate (5 mM) and subsaturating levels of glutamate (1 mM) to facilitate full activation of complex II by oxaloacetate removal. In this experimental design (outlined in Lambert et al, 2008), electrons from succinate reduce the ubiquinone pool via complex II and generate a proton motive force. These conditions allow electron and proton fluxes through complex Ito reverse and be directed predominantly toward the matrix. Addition of succinate causes a rapid increase in the reduction state of the matrix NAD(P)H pool that can be monitored as described above. The rate of NAD(P)H reduction is therefore a measure of electron flux through complex I for which direct comparisons can be made under identical conditions to effects on rates of ROS production. Candidate IQ ROS inhibitors were tested over a range of concentrations against rotenone or FCCP to evaluate relative effects of each on the rate of NAD(P)H reduction and the amount of observed ROS production. Ideal candidates may be distinguished from both these types of known mitochondrial inhibitors and display a range of concentrations over which IQ ROS production was significantly inhibited without any concomitant inhibition to the maximal reverse electron flux through complex I.

### Example 18

### Measurement of Cytochrome b₅₆₆ Reduction

The rates of ROS production from site IQ, site IIIQo, GPDH, and likely SDH are linked to the reduction state of the mobile ubiquinone pool in the mitochondrial inner membrane (Treberg et al, 2011; Quinlan, C. et al, (2011) The Mechanism of Superoxide Production by the Antimycin-inhibited Mitochondrial Q-cycle. J. Biol. Chem. 286(36): 31361-72; Orr et al, in revision). The reduction state of cytochrome b₅₆₆ in complex III can be used as a proxy for the reduction state of the ubiquinone pool (Quinlan et al., 2011) and is readily measured in isolated mitochondria (1.5 mg/mL) as the absorbance difference at the wavelength pair 566 nm and 575 nm on an Olis DW-2 dual wavelength spectrophotometer. Candidate compounds can be evaluated for effects on b₅₆₆ reduction to determine if their effects on various sites of ROS production are simply due to an altered reduction state of the ubiquinone pool. Ideally, a candidate compound would lower ROS at a specific site without altering b₅₆₆ reduction.

### Example 19

### Measurement of ΔΨm with TPMP-Sensitive Electrodes

ΔΨm can be calibrated in millivolts using TPMP-sensitive electrodes (Affourtit et al., 2012). This method of ΔΨm measurement is more accurate than fluorescence based TMRM or Safranin O methods. Because IQ ROS production is strongly influenced by the proton motive force, calibrated TPMP-based measurements of ΔΨm were made as final validation of candidate IQ ROS inhibitors to verify the absence of subtle effects of these inhibitors on proton motive force. Calibrations were made in a water-jacketed chamber at 37° C. with continuous stirring. Measurements were made in the presence and absence of nigercin using complex I (glutamate and malate) substrates in the presence of phosphate (5 mM) and magnesium (2 mM). Parallel measurements were made with succinate (5 mM) in standard buffer with and without nigericin. Ideally, a candidate would have no measurable effect on either the ΔpH (nigercin-sensitive) or ΔΨm components of the proton motive force.

### Example 20

### Measurement of Mitochondrial GPDH Activity

The activity of GPDH in intact mitochondria (0.3 mg/mL) was measured at 30° C. in standard buffer with 250 nM free calcium as the rate of reduction of 50 μM 2,6-dichlorophenolindophenol (DCPIP) by 0-60 mM glycerol phosphate as described previously (Dawson, A. and Thorne, J. (1969) The Reaction of Mitochondrial L-3-Glycerophosphate Dehydrogenase with Various Electron Acceptors. Biochem. J. 114(1): 35-40) except for the inclusion of 4 μM rotenone, 2 μM myxothiazol, 2.5 μM antimycin A, and 1 mM sodium cyanide and the miniaturization into microplate format. Candidate GPDH inhibitors were tested at varying concentrations up to 200 uM in order to determine the form of enzyme inhibition, inhibitor constants, IC50 values.

### Example 21

### Measurement of Cytosolic GPDH Activity

The specificity of inhibitors for mitochondrial GPDH versus the soluble cytosolic isoform was tested using commercially available soluble GPDH from rabbit muscle and following manufacturer protocols. Briefly, the rate of NADH oxidation by soluble GPDH in the presence of dihydroxyacetone phosphate and either DMSO or candidate compounds was determined using a Varian Cary Eclipse fluorimeter (ex 365/em 450 nm).

### ENUMERATED EMBODIMENTS

### Embodiment 1

A method of reducing oxidative stress in an individual in need thereof, comprising administering to the individual an effective amount of a compound capable of ROS inhibition or a pharmaceutically acceptable salt thereof, wherein the compound is selected from the group consisting of compounds 1-85, or a pharmaceutically acceptable salt thereof.

### Embodiment 2

A method of reducing oxidative stress in an individual in need thereof, comprising administering to the individual an effective amount of a compound capable of ROS inhibition, or a pharmaceutically acceptable salt thereof, wherein the compound is of the formula (I):

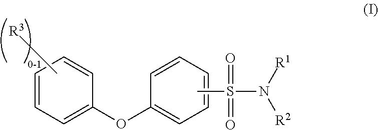

wherein:
R¹ and R² are independently selected from the group consisting of H, unsubstituted C₁-C₆ alkyl, —N═C(H)(aryl), —NH₂, unsubstituted aryl, substituted aryl or may be taken together with the nitrogen to which they are attached to form a nitrogen containing heterocyclic ring that may contain one additional heteroatom; and
R³ is selected from the group consisting of nitro, halo and C₁-C₆alkoxy;
   or a pharmaceutically acceptable salt thereof.

### Embodiment 3

A method of reducing oxidative stress in an individual in need thereof, comprising administering to the individual an effective amount of a compound capable of ROS inhibition, or a pharmaceutically acceptable salt thereof, wherein the compound is either:

(a) of the formula (II):

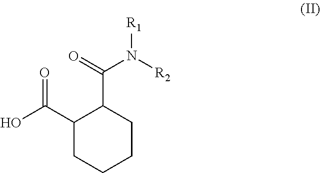

wherein R¹ and R² are independently H or a C₃-C₈cycloalkyl,

or

(b) of the formula (III):

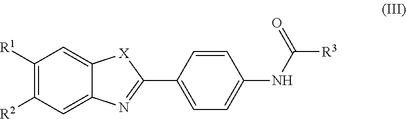

wherein:
R¹ and R² are independently selected from the group consisting of H, unsubstituted C₁-C₆ alkyl, C(═O)phenyl, phenyl, or may be taken together to form an aromatic ring;
R³ is selected from the group consisting of C₁-C₆ alkyl, carboxyl, and phenyl substituted with C₁-C₆ alkoxy; and
X is selected from the group NH, S and O,
   or a pharmaceutically acceptable salt thereof.

### Embodiment 4

A method of inhibiting ROS production in an individual in need thereof, comprising administering to the individual an effective amount of a compound capable of ROS inhibition or a pharmaceutically acceptable salt thereof, wherein the compound is selected from the group consisting of compounds 1-85, or a pharmaceutically acceptable salt thereof.

### Embodiment 5

A method of inhibiting ROS production in an individual in need thereof, comprising administering to the individual an effective amount of a compound capable of ROS inhibition, or a pharmaceutically acceptable salt thereof, wherein the compound is of the formula (I):

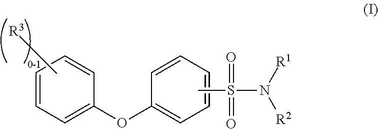

wherein:
R¹ and R² are independently selected from the group consisting of H, unsubstituted C₁-C₆ alkyl, —N═C(H)(aryl), —NH₂, unsubstituted aryl, substituted aryl or may be taken together with the nitrogen to which they are attached to form a nitrogen containing heterocyclic ring that may contain one additional heteroatom; and
R³ is selected from the group consisting of nitro, halo and C₁-C₆alkoxy;
   or a pharmaceutically acceptable salt thereof.

### Embodiment 6

A method of inhibiting ROS production in an individual in need thereof, comprising administering to the individual an effective amount of a compound capable of ROS inhibition, or a pharmaceutically acceptable salt thereof, wherein the compound is either:

(a) of the formula (II):

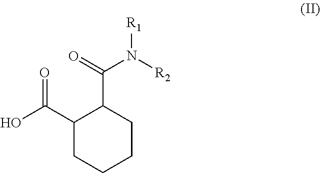

wherein R¹ and R² independently H or a C3-C8cycloalkyl, or (b) of the formula (III):

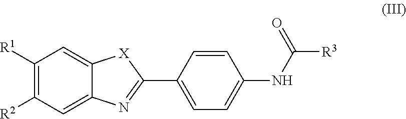

wherein:

R¹ and R² are independently selected from the group consisting of H, unsubstituted C₁-C₆ alkyl, C(═O)phenyl, phenyl, or may be taken together to form an aromatic ring;

R³ is selected from the group consisting of C₁-C₆ alkyl, carboxyl, and phenyl substituted with C₁-C₆ alkoxy; and

X is selected from the group NH, S and O,

or a pharmaceutically acceptable salt thereof.

### Embodiment 7

The method of any one of embodiments 1-6, wherein the compound selectively reduces ROS production at one of the ROS production sites IQ, IF, IIIQo, SDH or GPDH by at least 20% and reduces ROS production at the remaining ROS production sites IQ, IF, IIIQo, SDH and GPDH by no more than 10%.

### Embodiment 8

The method of any one of embodiments 1-6, wherein the compound selectively reduces ROS production at one of the ROS production sites IQ, IF, IIIQo, SDH or GPDH by at least 18% and reduces ROS production at the remaining ROS production sites IQ, IF, IIIQo, SDH and GPDH by no more than 12%.

### Embodiment 9

The method of any one of embodiments 1-6, wherein the compound selectively reduces ROS production at one of the ROS production sites IQ, IF, IIIQo, SDH or GPDH to a greater extent than it reduces ROS production at the remaining ROS production sites IQ, IF, IIIQo, SDH and GPDH.

### Embodiment 10

The method of any one of embodiments 1-6, wherein the compound selectively reduces ROS production at one of the ROS production sites IQ, IF, IIIQo, SDH or GPDH at least about 2-fold or greater than its ability to reduce ROS production at the remaining ROS production sites IQ, IF, IIIQo, SDH and GPDH.

### Embodiment 11

The method of any one of embodiments 1-6, wherein the compound selectively reduces ROS production at one of the ROS production sites IQ, IF, IIIQo, SDH or GPDH at least about 1.5-fold or greater than its ability to reduce ROS production at the remaining ROS production sites IQ, IF, IIIQo, SDH and GPDH.

### Embodiment 12

The method of any one of embodiments 1-11, wherein the compound selectively reduces ROS production at ROS production site IQ over ROS production sites IF, IIIQo, SDH and GPDH.

### Embodiment 13

The method of embodiment 12, wherein the compound is selected from the group consisting of:

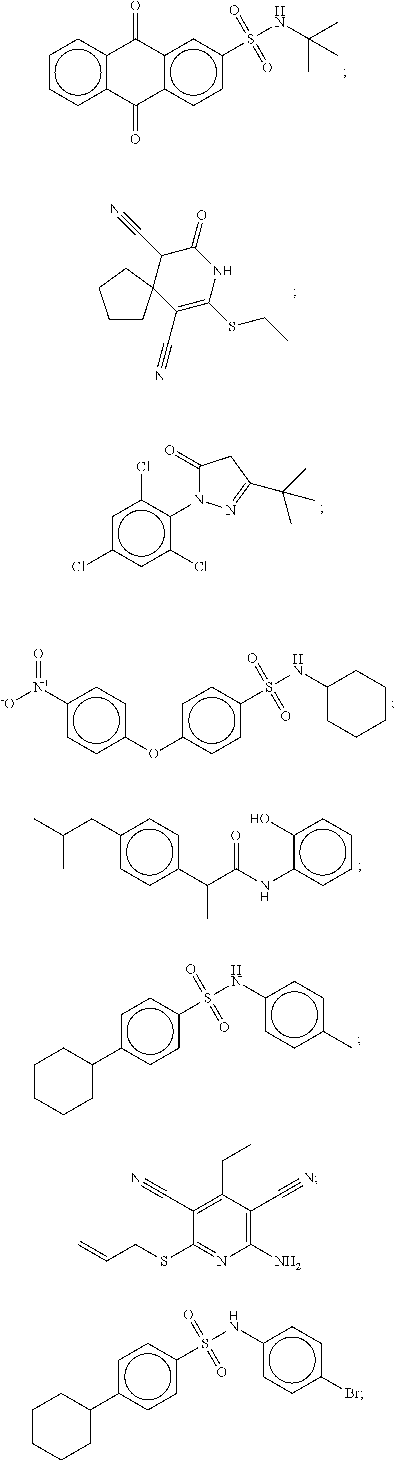

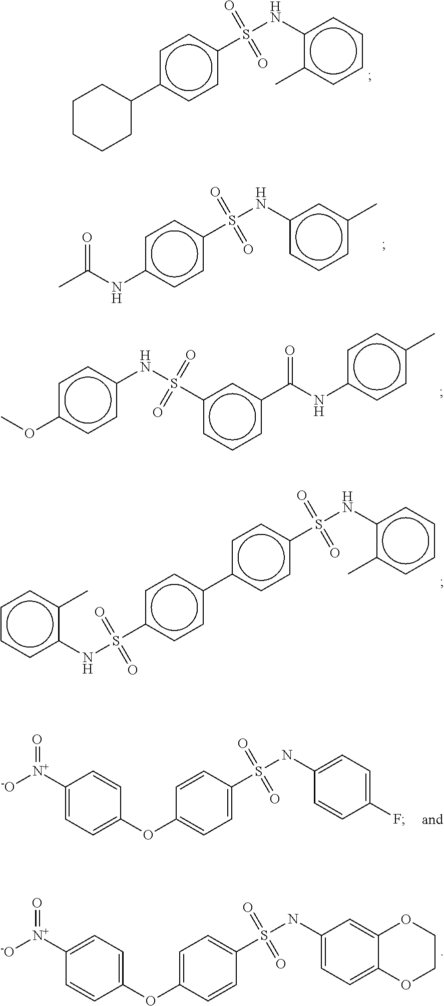

### Embodiment 14

The method of any one of embodiments 1-11, wherein the compound selectively reduces ROS production at ROS production site IF over ROS production sites IQ, IIIQo, SDH and GPDH.

### Embodiment 15

The method of embodiment 14, wherein the compound is selected from the group consisting of:

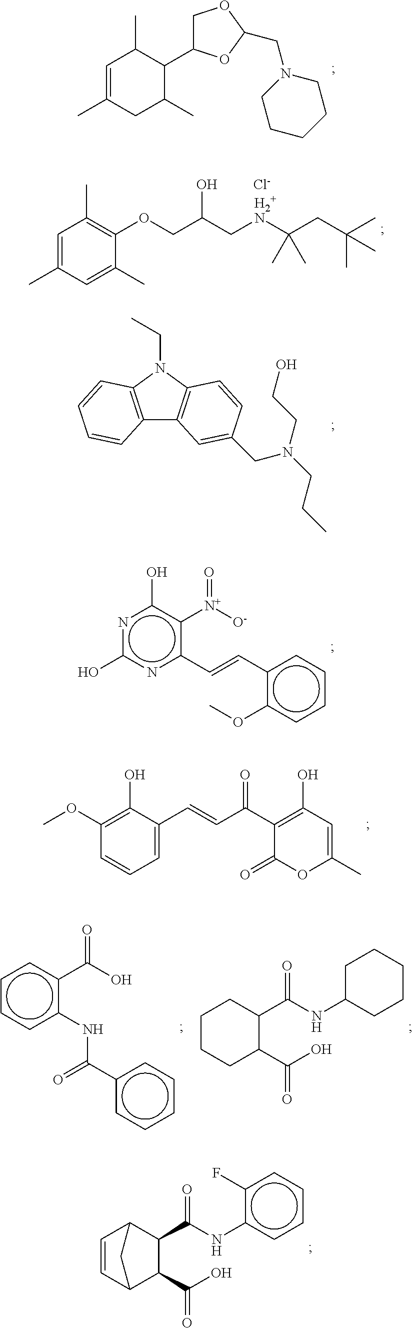

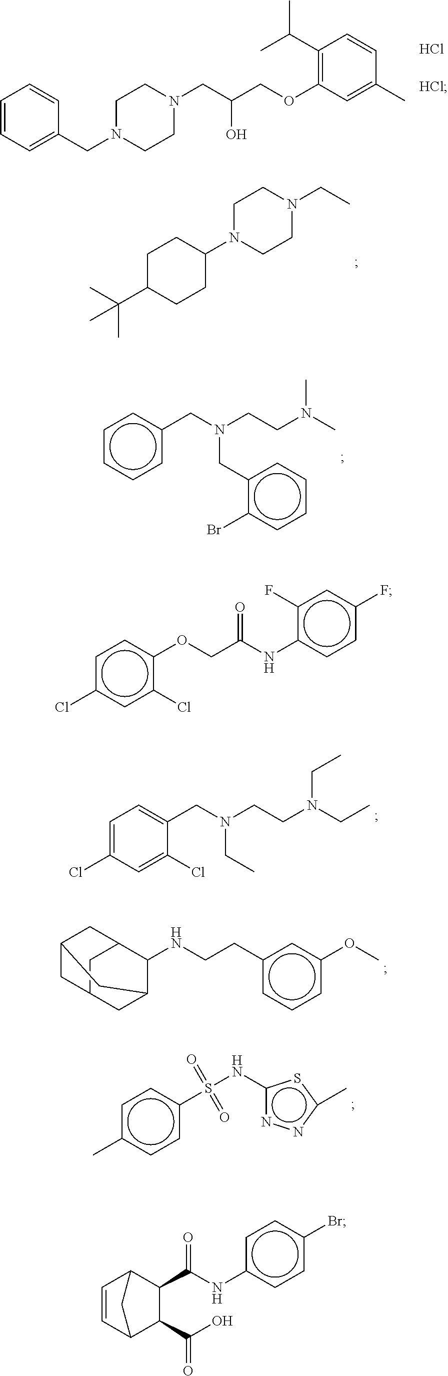

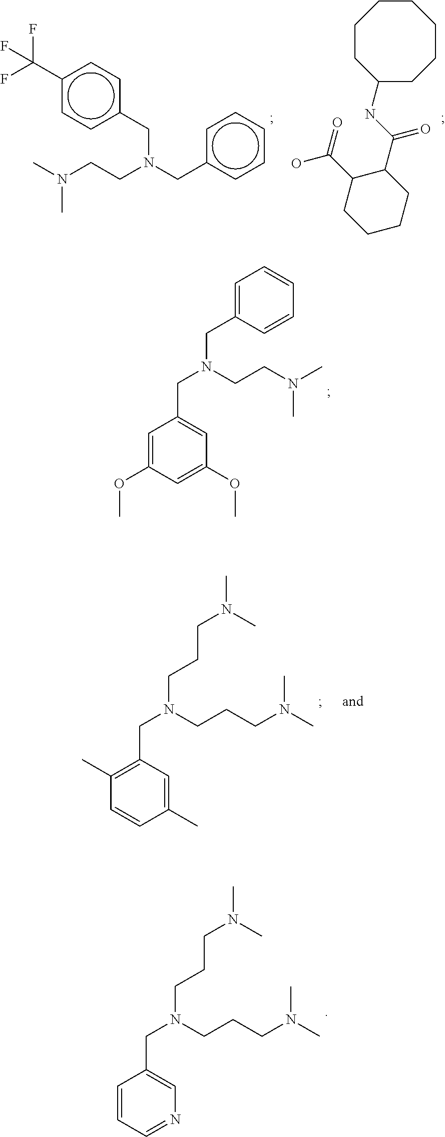

### Embodiment 16

The method of any one of embodiments 1-11, wherein the compound selectively reduces ROS production at ROS production site IIIQo over ROS production sites IQ, IF, SDH and GPDH.

### Embodiment 17

The method of embodiment 16, wherein the compound is selected from the group consisting of:

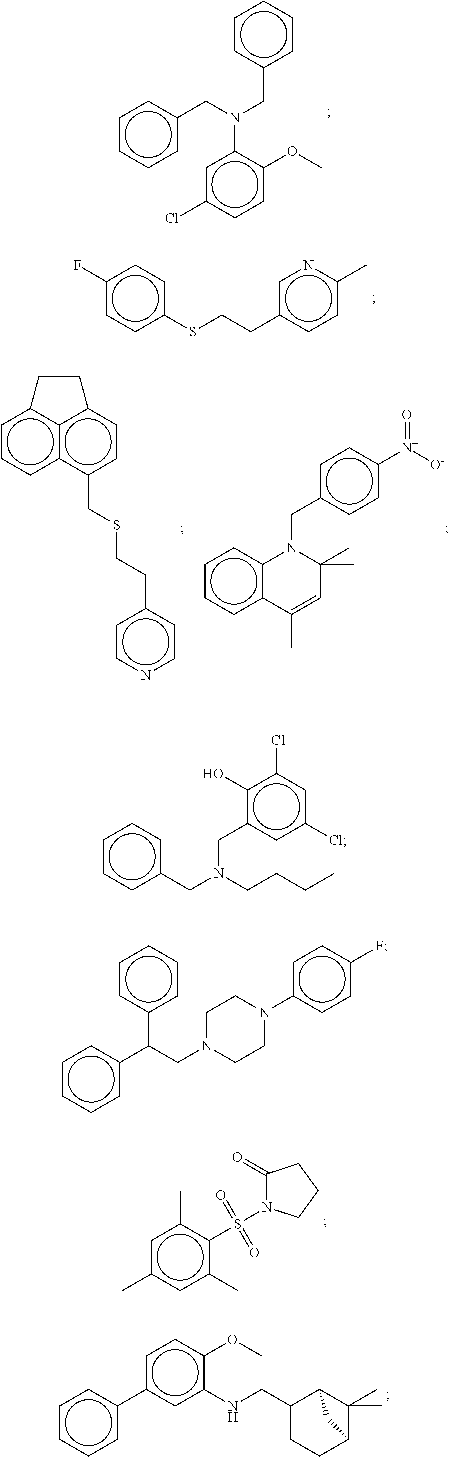

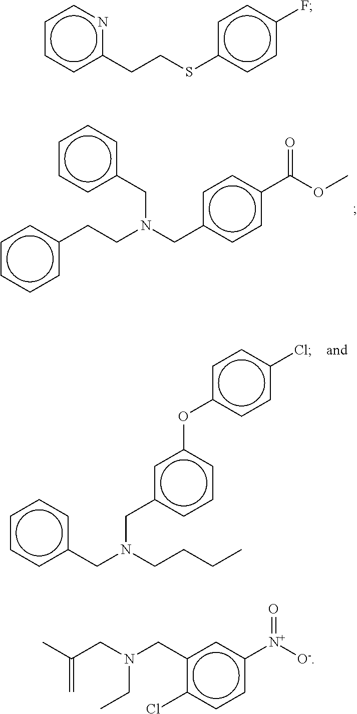

### Embodiment 18

The method of any one of embodiments 1-11, wherein the compound selectively reduces ROS production at ROS production site SDH over ROS production sites IQ, IF, IIIQo and GPDH.

### Embodiment 19

The method of embodiment 18, wherein the compound is selected from the group consisting of:

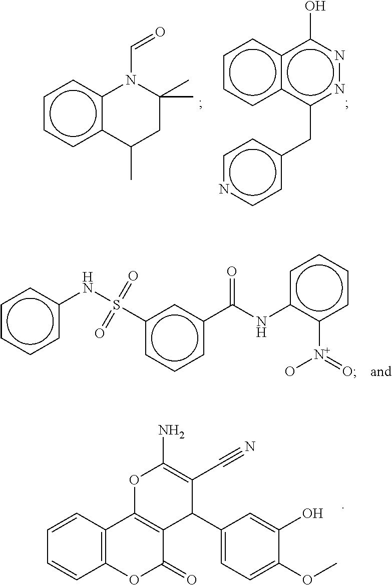

### Embodiment 20

The method of any one of embodiments 1-11 wherein the compound selectively reduces ROS production at ROS production site GPDH over ROS production sites IQ, IF, IIIQo and SDH.

### Embodiment 21

The method of embodiment 20, wherein the compound is selected from the group consisting of:

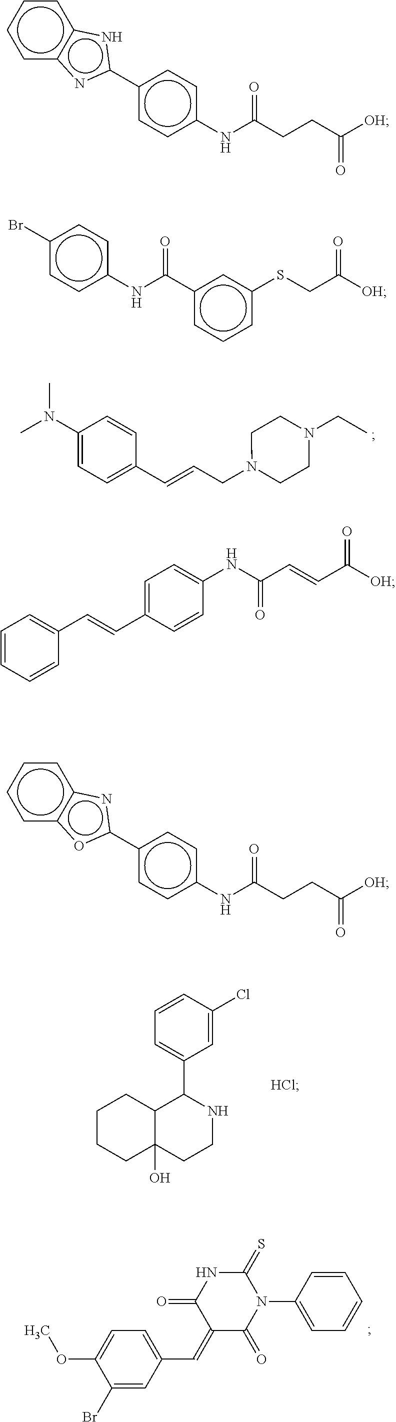

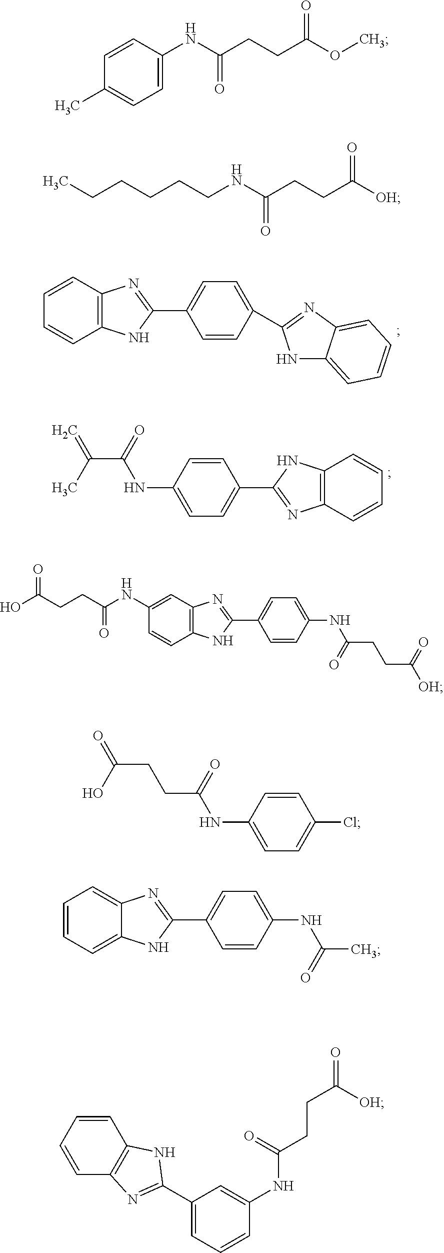

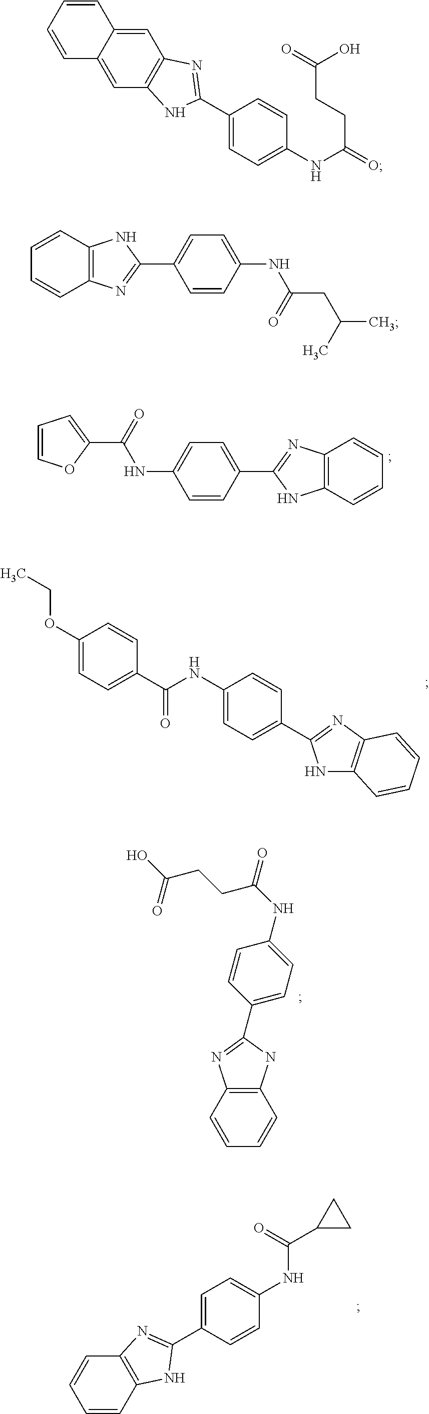 [SMILES: O=C(O)CCC(=O)Nc1ccc(-c2nc3cc4ccccc4cc3[nH]2)cc1 ; CC(C)CC(=O)Nc1ccc(-c2nc3ccccc3[nH]2)cc1 ; O=C(Nc1ccc(-c2nc3ccccc3[nH]2)cc1)c1ccco1 ; CCOc1ccc(C(=O)Nc2ccc(-c3nc4ccccc4[nH]3)cc2)cc1 ; O=C(O)CCC(=O)Nc1ccc(-c2nc3ccccc3[nH]2)cc1 ; O=C(Nc1ccc(-c2nc3ccccc3[nH]2)cc1)C1CC1]

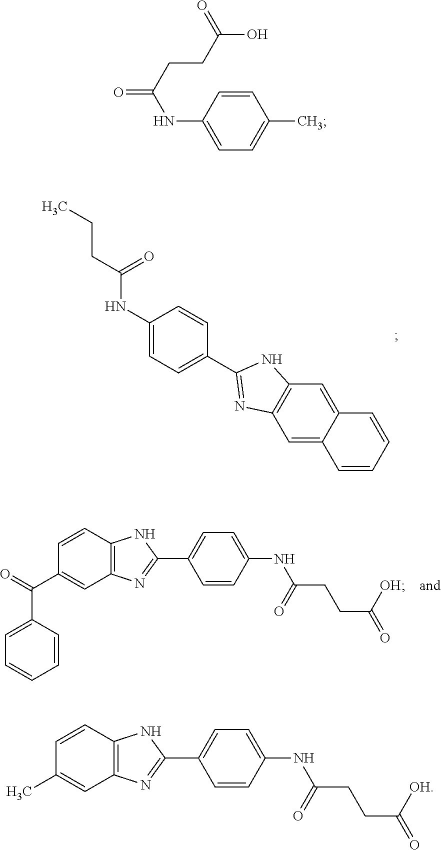 [SMILES: Cc1ccc(NC(=O)CCC(=O)O)cc1 ; CCCC(=O)Nc1ccc(-c2nc3cc4ccccc4cc3[nH]2)cc1 ; O=C(O)CCC(=O)Nc1ccc(-c2nc3cc(C(=O)c4ccccc4)ccc3[nH]2)cc1 ; "and" ; Cc1ccc2[nH]c(-c3ccc(NC(=O)CCC(=O)O)cc3)nc2c1]

### Embodiment 22

The method of any one of embodiments 1-21, wherein the compound reduces membrane potential by no more than 4%.

### Embodiment 23

The method of any one of embodiments 1-22 wherein the individual has or is suspected of having a disease or condition in which ROS is implicated.

### Embodiment 24

The method of embodiment 23, wherein the disease or condition is selected from the group consisting of atherosclerosis, heart disease, heart failure, hypertension, sepsis, diabetes, Alzheimer's disease, Parkinson's disease, toxin-induced parkinsonism, Huntington's disease, Wilson's disease, Friedreich's Ataxia, Kearns-Sayre syndrome, Leigh syndrome, Leber hereditary optic neuropathy, mitochondrial myopathy, cardiomyopathy, deafness, mood disorders, movement disorders, dementia, Amyotropic Lateral Sclerosis, Multiple Sclerosis, tardive dyskinesia, brain injury, schizophrenia, epilepsy, AIDS dementia, endothelial nitroglycerin tolerance, adriamycin toxicity, kidney damage in type I diabetes, kidney preservation ex vivo, cocaine toxicity, alcohol fatty liver disease, fatty liver disease, liver inflammation in hepatitis C virus patients, neuroprotection, immobilization-induced muscle atrophy, skeletal muscle burn injury, cancer, inflammation and ischemic-reperfusion injury in stroke, heart attack, UV damage to the skin, and during organ transplantation and surgery.

### Embodiment 25

The method of embodiment 24 wherein the individual has or is suspected of having cancer.

### Embodiment 26

The method of embodiment 24 wherein the individual has or is suspected of having diabetes.

### Embodiment 27

The method of embodiment 24 wherein the individual has or is suspected of having an ischemia/reperfusion injury.

### Embodiment 28

The method of embodiment 27, wherein the ischemia/reperfusion injury is a cerebral or cardiac ischemia reperfusion injury.

### Embodiment 29

The method of embodiment 27, wherein the ischemia/reperfusion injury is stroke or a heart-attack.

### Embodiment 30

The method of embodiment 24 wherein the individual has or is suspected of having Alzheimer's disease.

### Embodiment 31

The method of embodiment 24, wherein the individual has or is suspected of having chronic inflammation.

### Embodiment 32

A pharmaceutical composition comprising a pharmaceutically acceptable carrier and a compound selected from the group consisting of compounds 1-85, or a pharmaceutically acceptable salt thereof.

### Embodiment 33

A pharmaceutical composition comprising a pharmaceutically acceptable carrier and a compound of the formula (I):

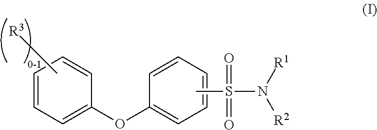

wherein:
R¹ and R² are independently selected from the group consisting of H, unsubstituted C₁-C₆ alkyl, —N═C(H)(aryl), —NH₂, unsubstituted aryl, substituted aryl or may be taken together with the nitrogen to which they are attached to form a nitrogen containing heterocyclic ring that may contain one additional heteroatom; and
R³ is selected from the group consisting of nitro, halo and C₁-C₆alkoxy;
   or a pharmaceutically acceptable salt thereof.

### Embodiment 34

A pharmaceutical composition comprising a pharmaceutically acceptable carrier and a compound either:

(a) of the formula (II):

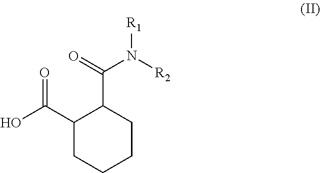

Wherein R¹ and R² are independently H or a C3-C8cycloalkyl,

or (b) of the formula (III):

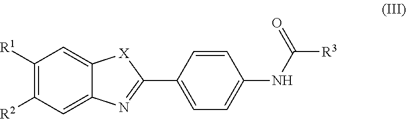

wherein:

R¹ and R² are independently selected from the group consisting of H, unsubstituted C₁-C₆ alkyl, C(═O)phenyl, phenyl, or may be taken together to form an aromatic ring;

R³ is selected from the group consisting of C₁-C₆ alkyl, carboxyl, and phenyl substituted with C₁-C₆ alkoxy; and

X is selected from the group NH, S and O,

or a pharmaceutically acceptable salt thereof.

### Embodiment 35

A method of identifying a compound capable of: (1) selectively inhibiting ROS production at one of the ROS production sites IQ, IF, IIIQ_{O}, SDH, or GPDH to a greater extent than the remaining ROS production sites IQ, IF, IIIQ_{O}, SDH, and GPDH and (2) decreasing mitochondrial membrane potential by no more than 4% relative to untreated mitochondria, wherein the method comprises the steps of contacting a cell or purified mitochondria with a compound and measuring ROS production at ROS production sites IQ, IF, IIIQ_{O}, SDH and GPDH and measuring membrane potential.

### Embodiment 36

The method of embodiment 35, wherein the compound decreases ROS production from one of the ROS production sites IQ, IF, IIIQ_{O}, SDH, or GPDH by at least about 20% and decreases ROS production from the remaining ROS production sites IQ, IF, IIIQ_{O}, SDH, and GPDH by less than about 10% while decreasing mitochondrial membrane potential by no more than 4% relative to untreated mitochondria.

### Embodiment 37

A method of identifying a compound capable of selectively inhibiting ROS production at one of the ROS production sites IQ, IF, IIIQ_{O}, SDH, or GPDH to a greater extent than the remaining ROS production sites IQ, IF, IIIQ_{O}, SDH, and GPDH, wherein the method comprises the steps of contacting a cell or purified mitochondria with a compound and measuring ROS production at ROS production sites IQ, IF, IIIQ_{O}, SDH and GPDH.

### Embodiment 38

A method of identifying a compound capable of selectively inhibiting enzymatic activity of one of the ROS production sites IQ, IF, IIIQ_{O}, SDH, or GPDH to a greater extent than the remaining ROS production sites IQ, IF, IIIQ_{O}, SDH, and GPDH, wherein the method comprises the steps of contacting a cell or purified mitochondria with a compound and measuring ROS production at ROS production sites IQ, IF, IIIQ_{O}, SDH and GPDH.

### Embodiment 39

A method of identifying a compound capable of: (1) inhibiting ROS production at one or more ROS production sites IQ, IF, IIIQ_{O}, SDH, or GPDH and (2) decreasing mitochondrial membrane potential by no more than 4% relative to untreated mitochondria, wherein the method comprises the steps of contacting a cell or purified mitochondria with a compound and measuring ROS production at ROS production sites IQ, IF, IIIQ_{O}, SDH and GPDH and measuring membrane potential.

All references throughout, such as publications, patents, patent applications and published patent applications, are incorporated herein by reference in their entireties.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A method of reducing oxidative stress or inhibiting ROS production in an individual in need thereof, comprising administering to the individual an effective amount of a compound capable of ROS inhibition or a pharmaceutically acceptable salt thereof, wherein the compound is selected from the group consisting of:

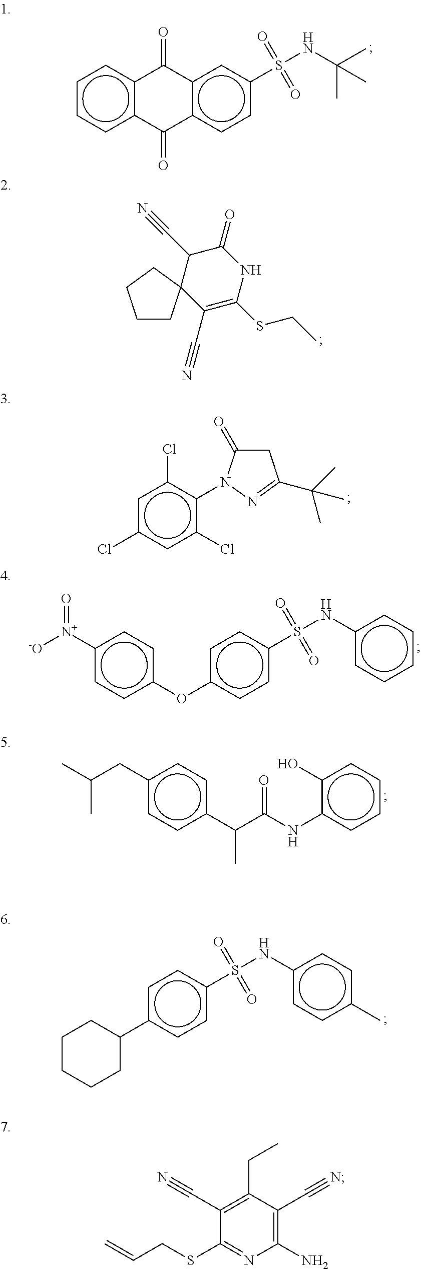

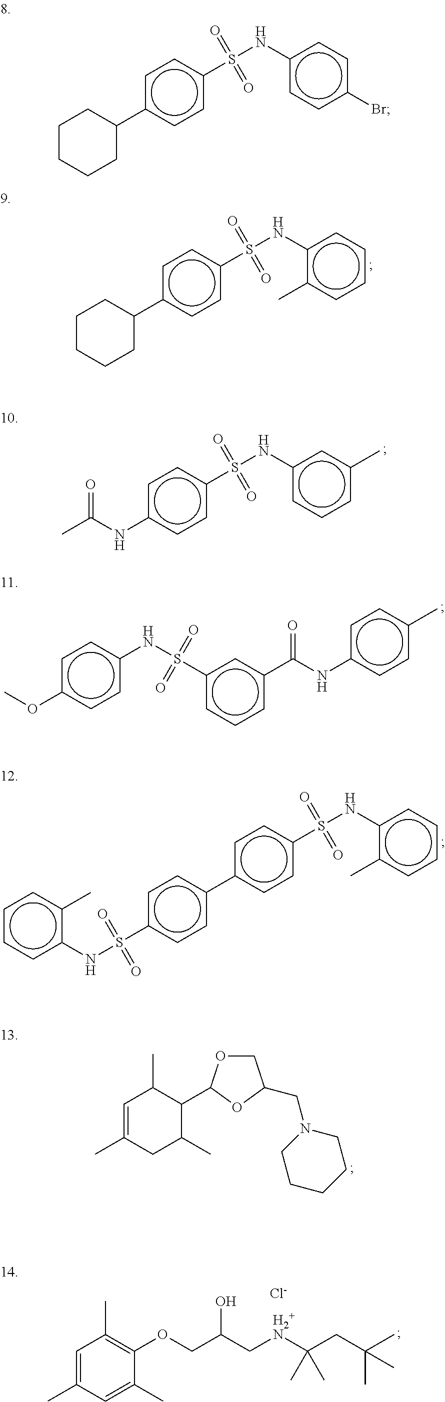

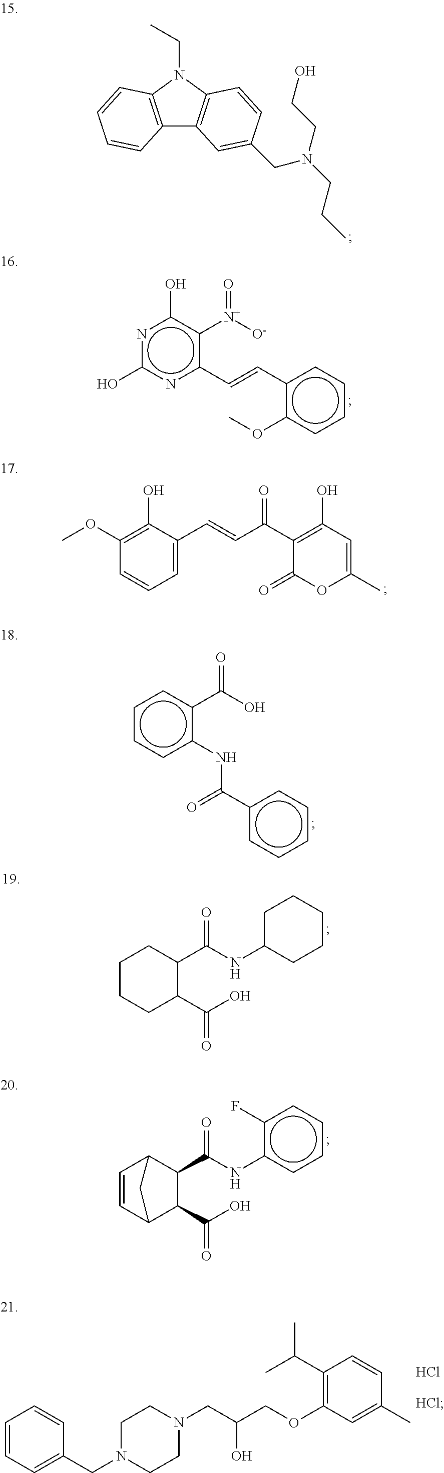

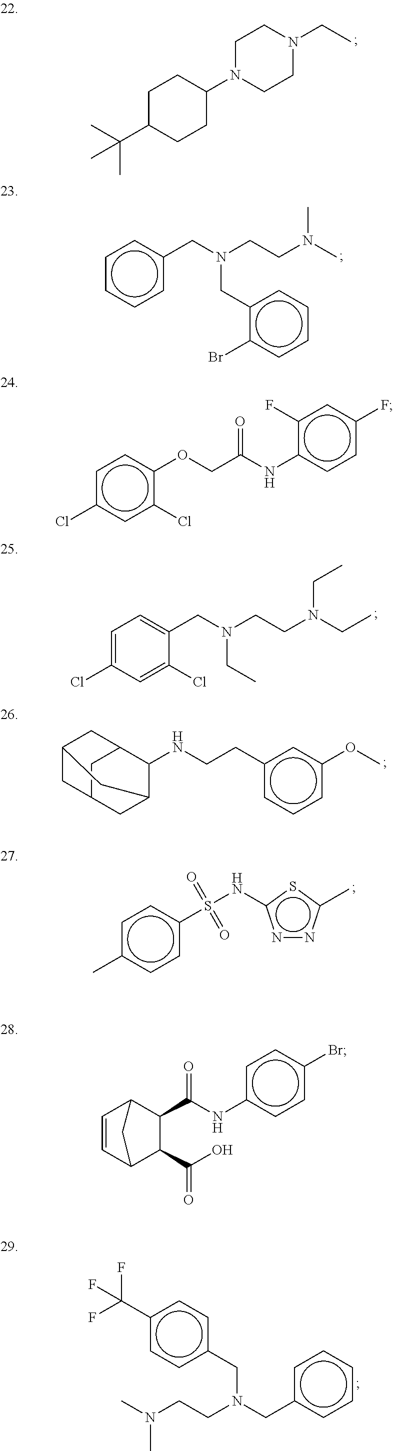

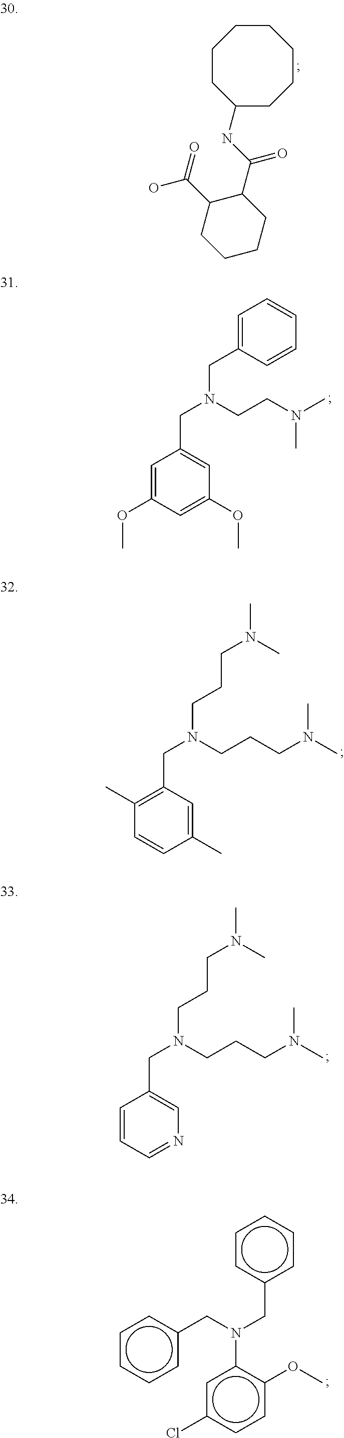

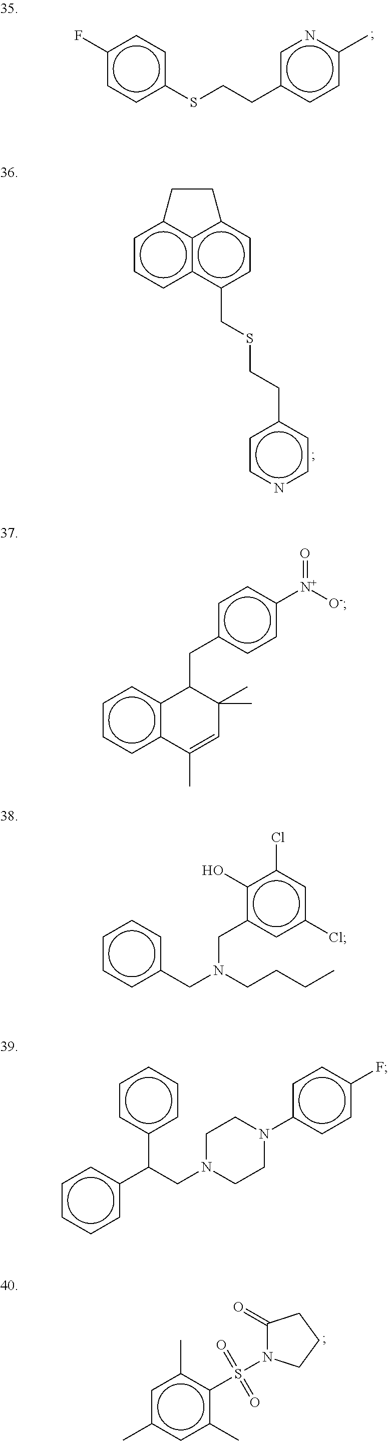

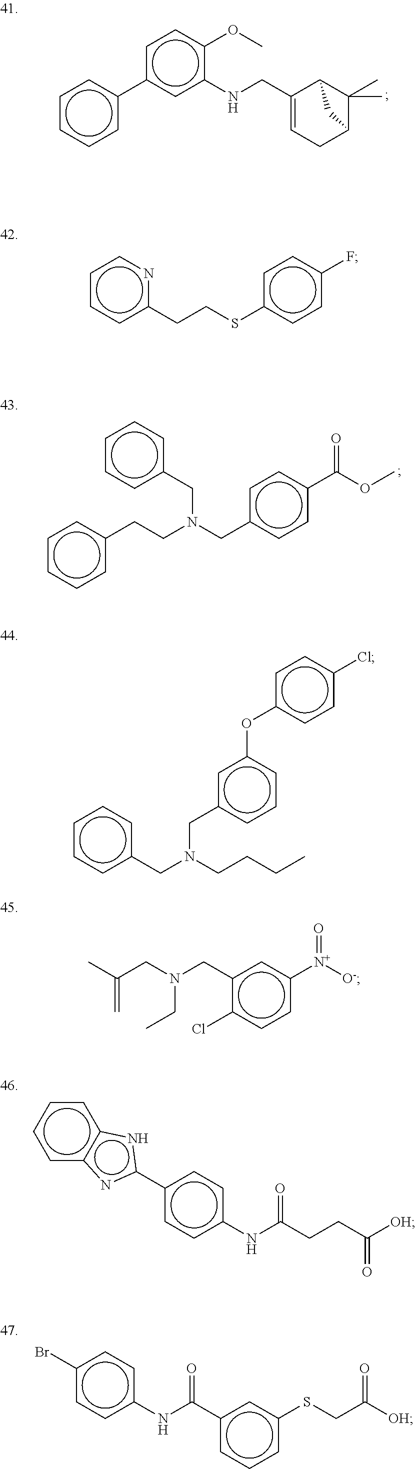

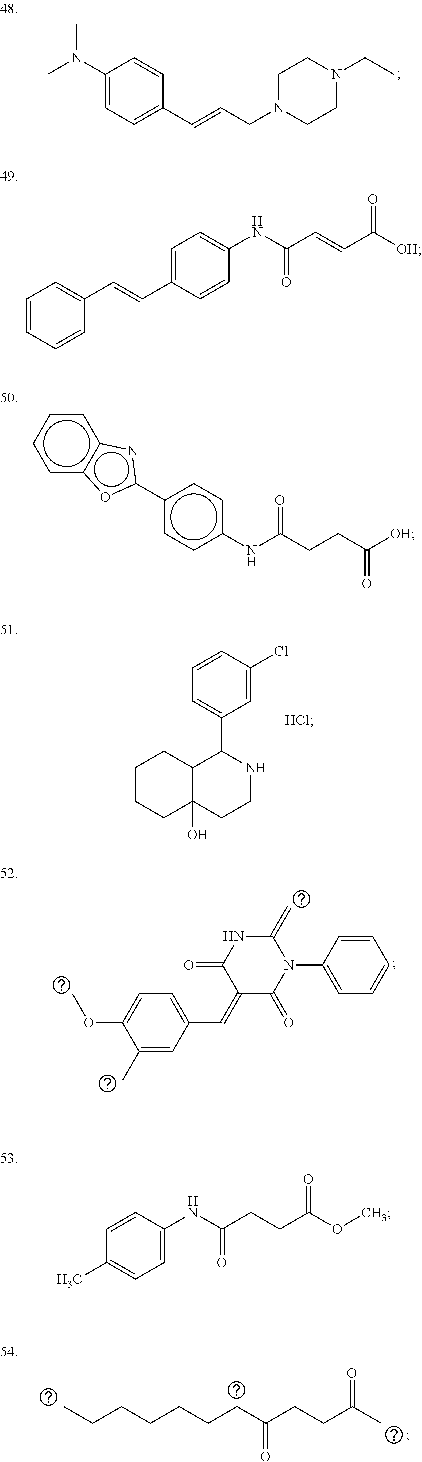

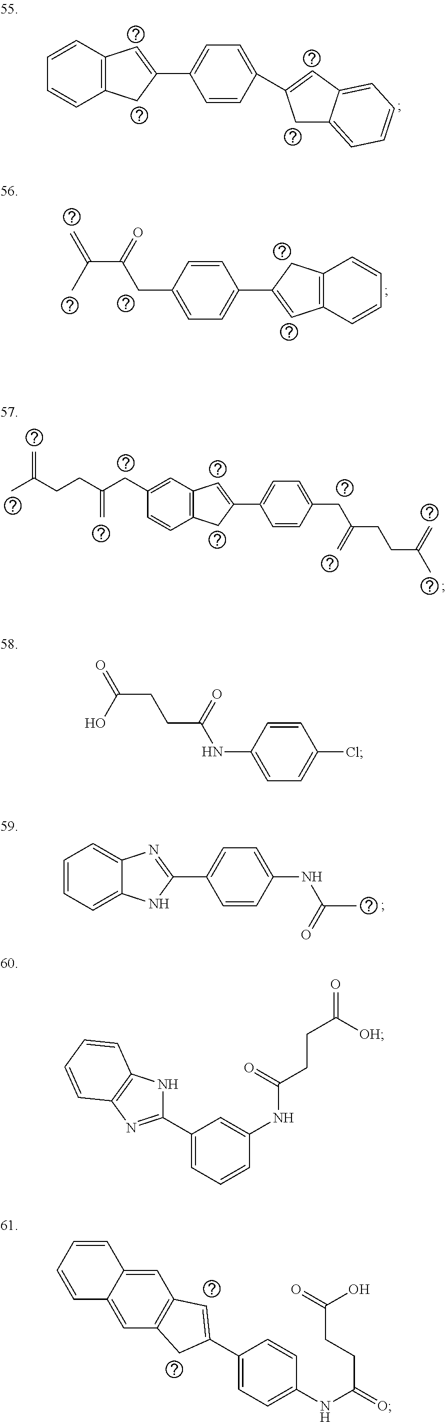

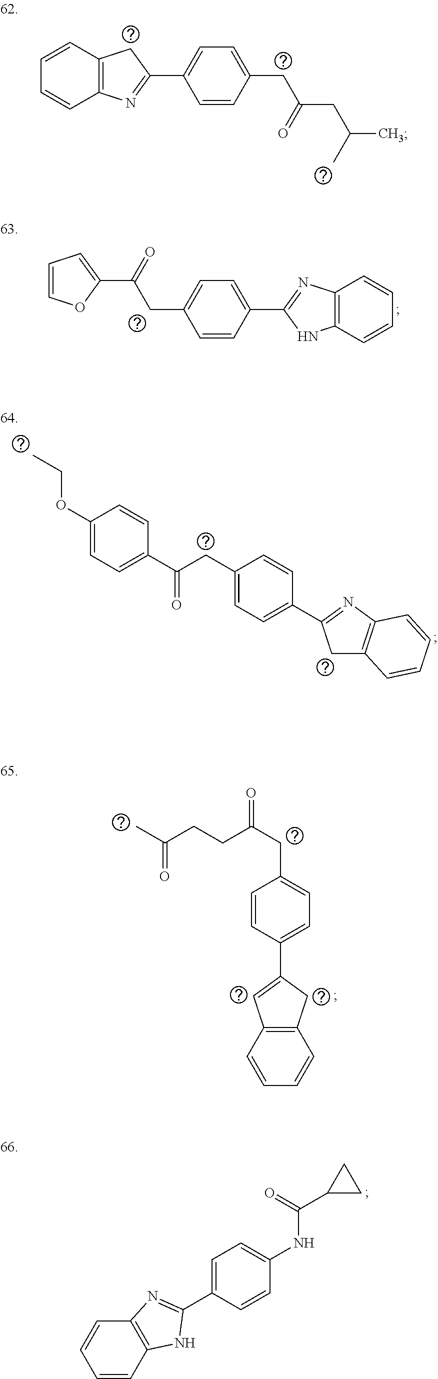 [SMILES: CC(C)CC(=O)Cc1ccc(C2=Nc3ccccc3C2)cc1 ; O=C(Cc1ccc(-c2nc3ccccc3[nH]2)cc1)c1ccco1 ; CCOc1ccc(C(=O)Cc2ccc(C3=Nc4ccccc4C3)cc2)cc1 ; CC(=O)CCC(=O)Cc1ccc(C2=Cc3ccccc3C2)cc1 ; O=C(Nc1ccc(-c2nc3ccccc3[nH]2)cc1)C1CC1]

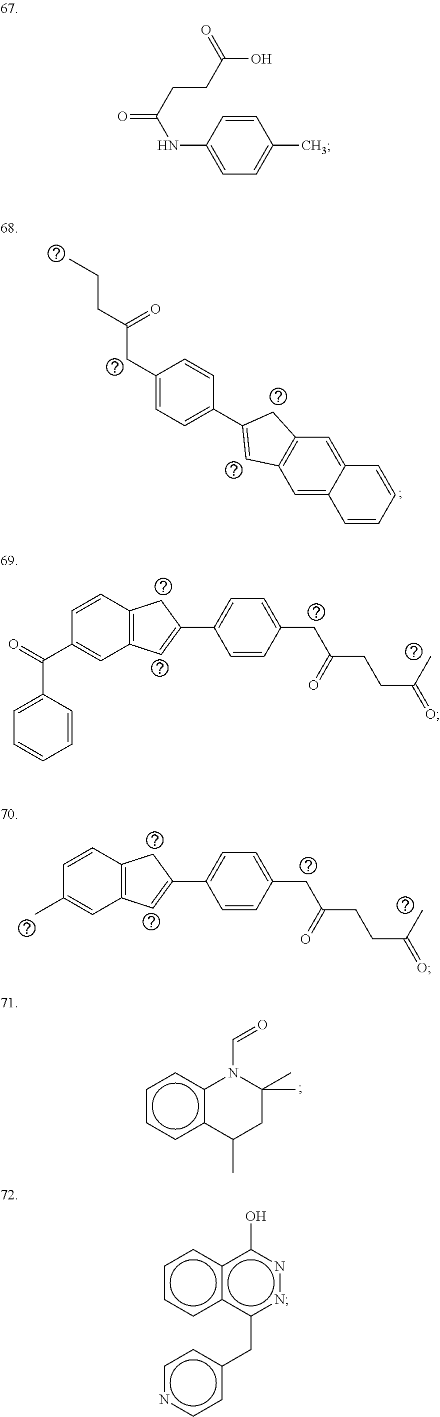

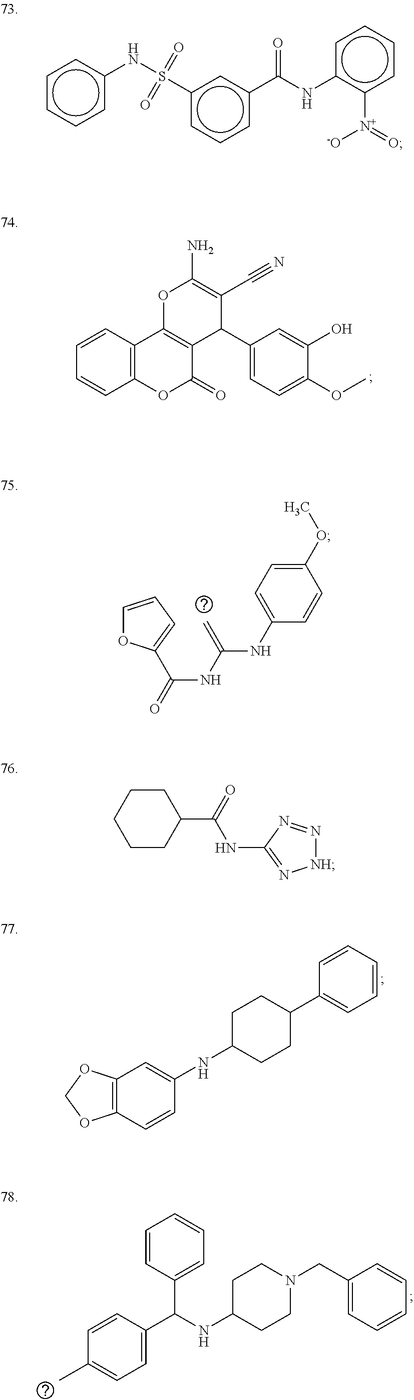

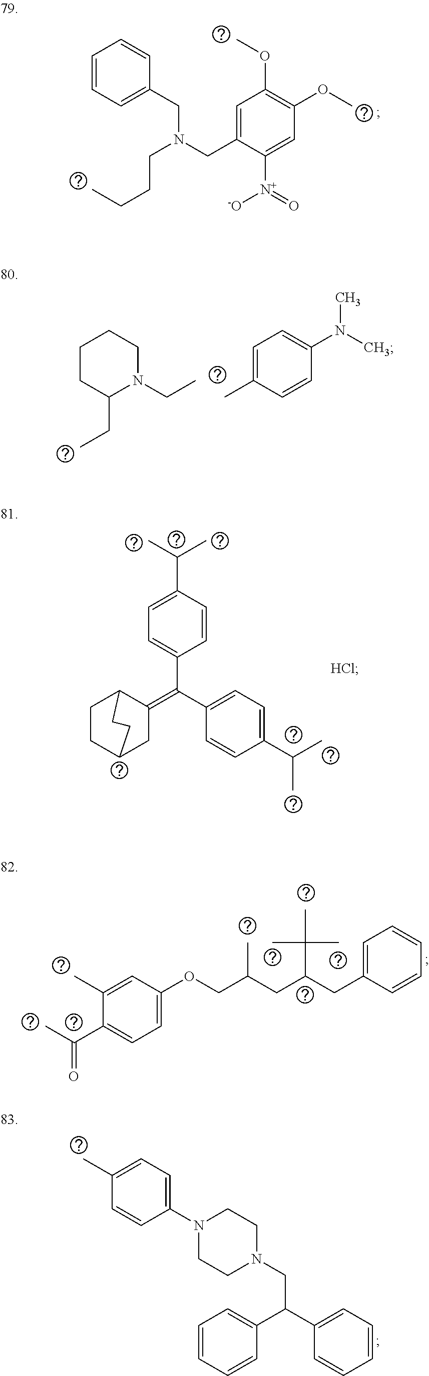

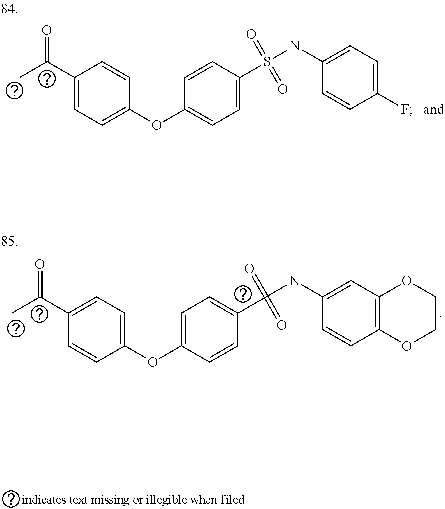

2. A method of reducing oxidative stress or inhibiting ROS production in an individual in need thereof, comprising administering to the individual an effective amount of a compound capable of ROS inhibition, or a pharmaceutically acceptable salt thereof, wherein the compound is of the formula (I): 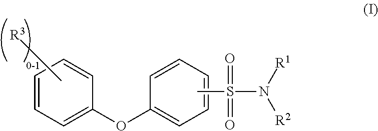
wherein:
R¹ and R² are independently selected from the group consisting of H, unsubstituted C₁-C₆ alkyl, —N═C(H)(aryl), —NH₂, unsubstituted aryl, substituted aryl or may be taken together with the nitrogen to which they are attached to form a nitrogen containing heterocyclic ring that may contain one additional heteroatom; and
R³ is selected from the group consisting of nitro, halo and C₁-C₆alkoxy;
or a pharmaceutically acceptable salt thereof.

3. A method of reducing oxidative stress or inhibiting ROS production in an individual in need thereof, comprising administering to the individual an effective amount of a compound capable of ROS inhibition, or a pharmaceutically acceptable salt thereof, wherein the compound is either:
(a) of the formula (II): 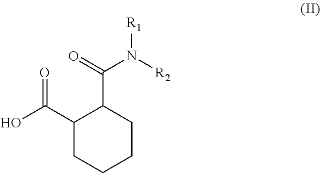
wherein R¹ and R² are independently H or a C₃-C₈cycloalkyl,
or
(b) of the formula (III): 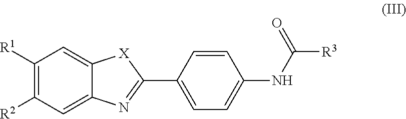
wherein:
R¹ and R² are independently selected from the group consisting of H, unsubstituted C₁-C₆ alkyl, C(═O)phenyl, phenyl, or may be taken together to form an aromatic ring;
R³ is selected from the group consisting of C₁-C₆ alkyl, carboxyl, and phenyl substituted with C₁-C₆ alkoxy; and
X is selected from the group NH, S and O,
or a pharmaceutically acceptable salt thereof.

4. The method of claim 1, wherein the compound selectively reduces ROS production at one of the ROS production sites IQ, IF, IIIQo, SDH or GPDH by at least 20% and reduces ROS production at the remaining ROS production sites IQ, IF, IIIQo, SDH and GPDH by no more than 10%.

5. The method of claim 1, wherein the compound selectively reduces ROS production at one of the ROS production sites IQ, IF, IIIQo, SDH or GPDH by at least 18% and reduces ROS production at the remaining ROS production sites IQ, IF, IIIQo, SDH and GPDH by no more than 12%.

6. The method of claim 1, wherein the compound selectively reduces ROS production at one of the ROS production sites IQ, IF, IIIQo, SDH or GPDH to a greater extent than it reduces ROS production at the remaining ROS production sites IQ, IF, IIIQo, SDH and GPDH.

7. The method of claim 1, wherein the compound selectively reduces ROS production at one of the ROS production sites IQ, IF, IIIQo, SDH or GPDH at least about 2-fold or greater than its ability to reduce ROS production at the remaining ROS production sites IQ, IF, IIIQo, SDH and GPDH.

8. The method of claim 1, wherein the compound selectively reduces ROS production at one of the ROS production sites IQ, IF, IIIQo, SDH or GPDH at least about 1.5-fold or greater than its ability to reduce ROS production at the remaining ROS production sites IQ, IF, IIIQo, SDH and GPDH.

9. The method of claim 1, wherein the compound selectively reduces ROS production at ROS production site IQ over ROS production sites IF, IIIQo, SDH and GPDH.

10. The method of claim 9, wherein the compound is selected from the group consisting of:

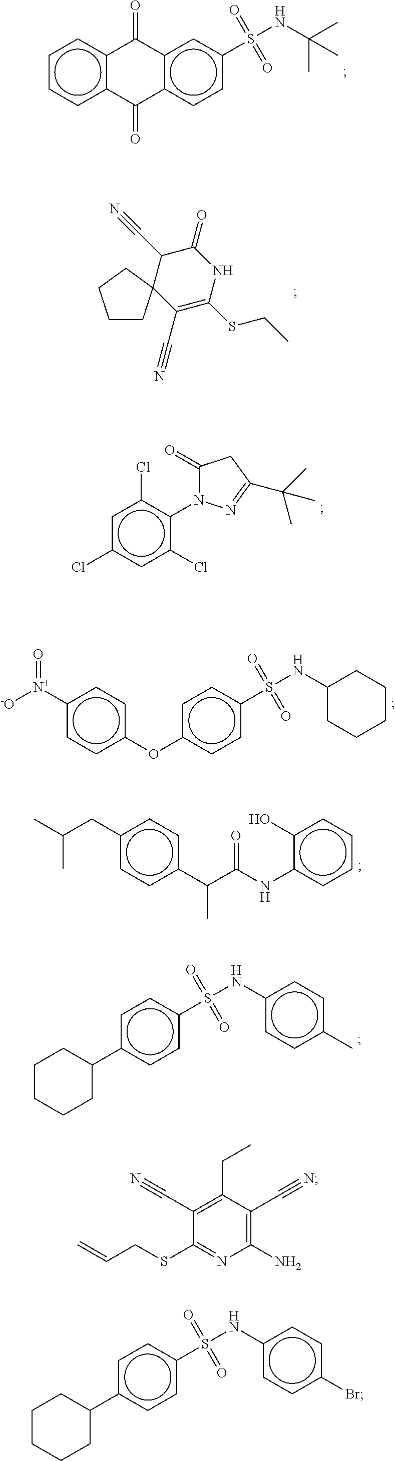

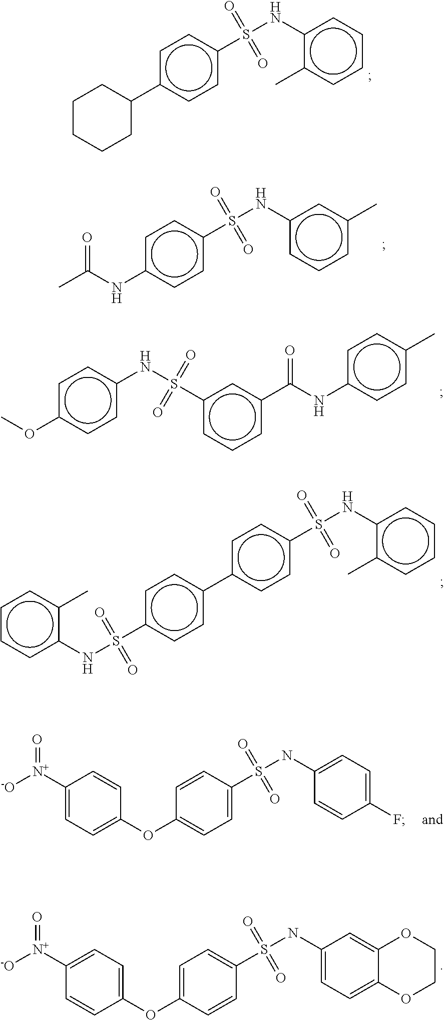

11. The method of claim 1, wherein the compound selectively reduces ROS production at ROS production site IF over ROS production sites IQ, IIIQo, SDH and GPDH.

12. The method of claim 11, wherein the compound is selected from the group consisting of:

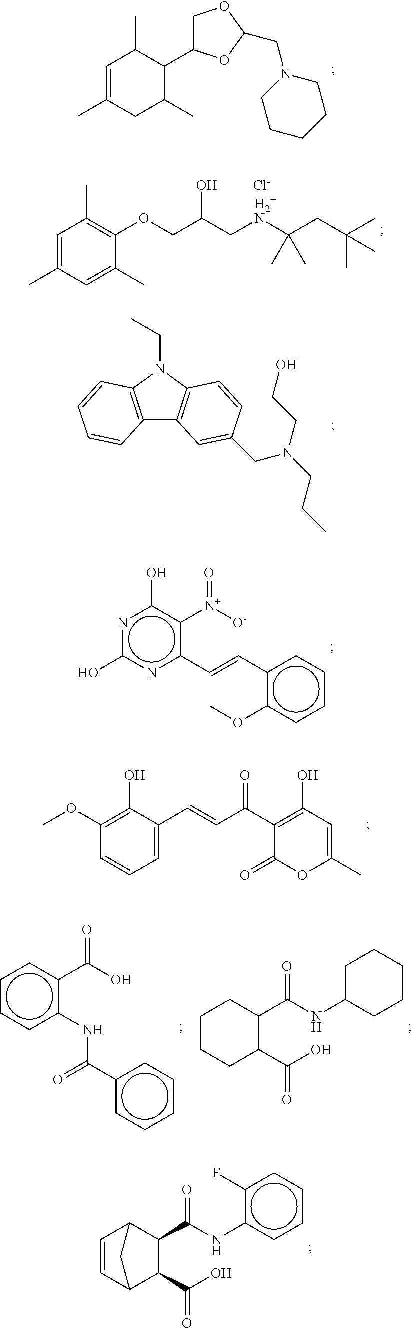

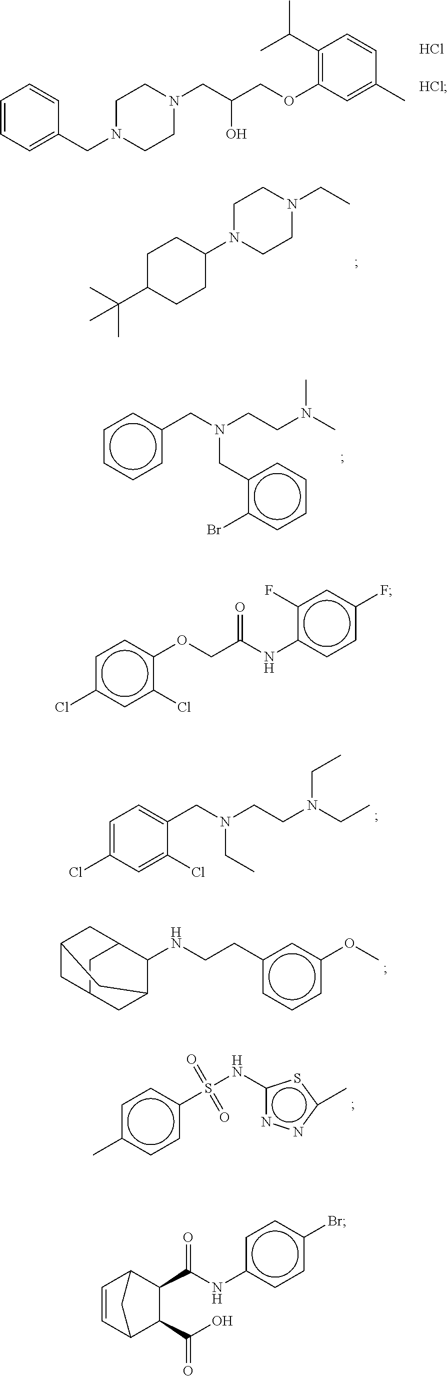

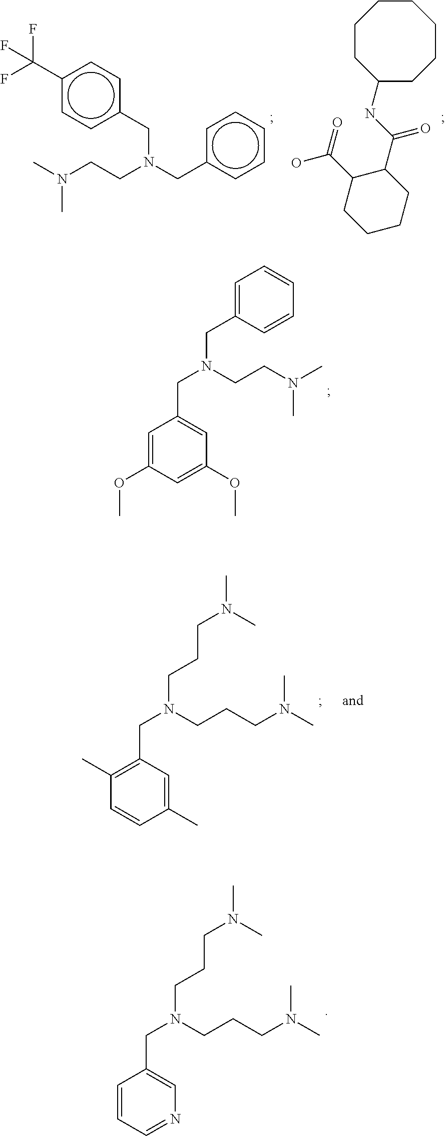

13. The method of claim 1, wherein the compound selectively reduces ROS production at ROS production site IIIQo over ROS production sites IQ, IF, SDH and GPDH.

14. The method of claim 13, wherein the compound is selected from the group consisting of:

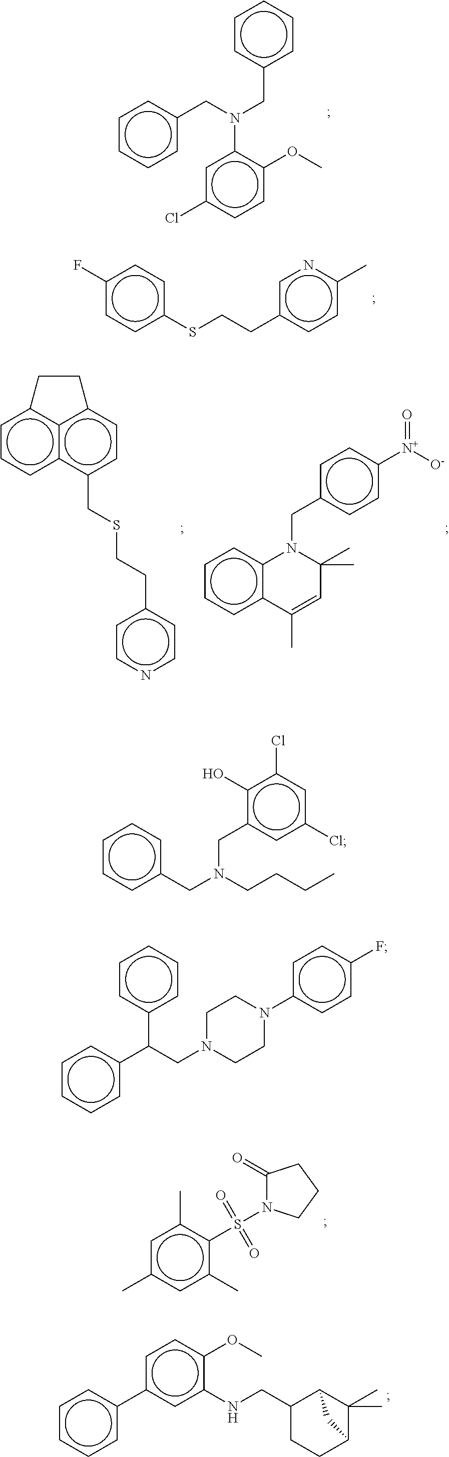

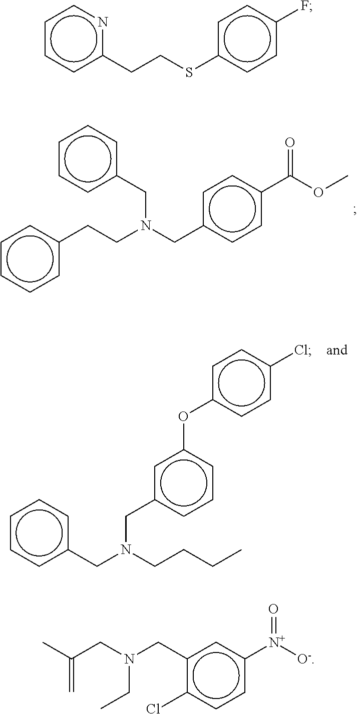

15. The method of claim 1, wherein the compound selectively reduces ROS production at ROS production site SDH over ROS production sites IQ, IF, IIIQo and GPDH.

16. The method of claim 15, wherein the compound is selected from the group consisting of:

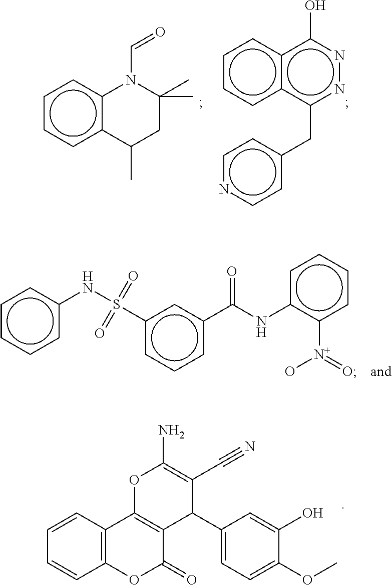

17. The method of claim 1, wherein the compound selectively reduces ROS production at ROS production site GPDH over ROS production sites IQ, IF, IIIQo and SDH.

18. The method of claim 17, wherein the compound is selected from the group consisting of:

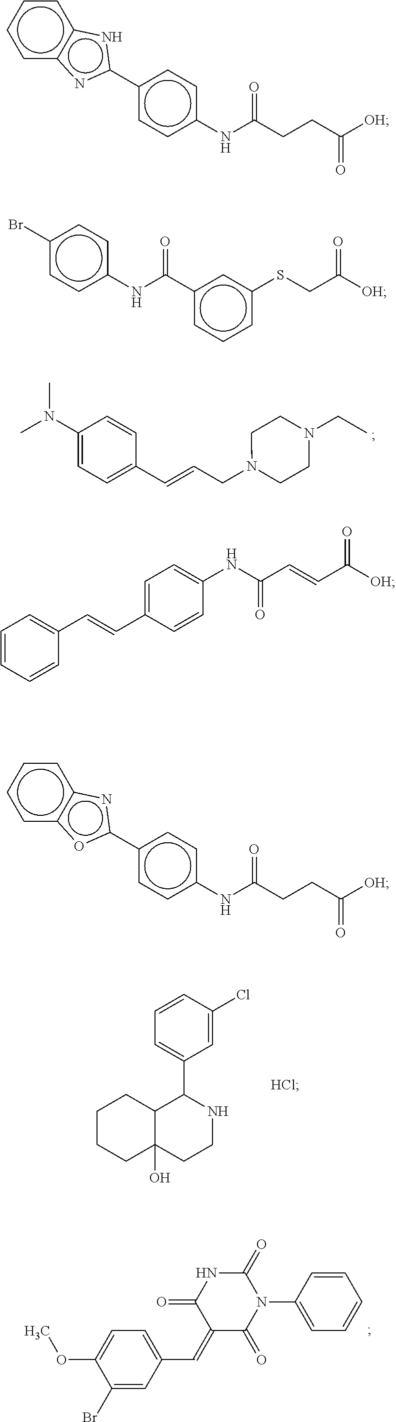

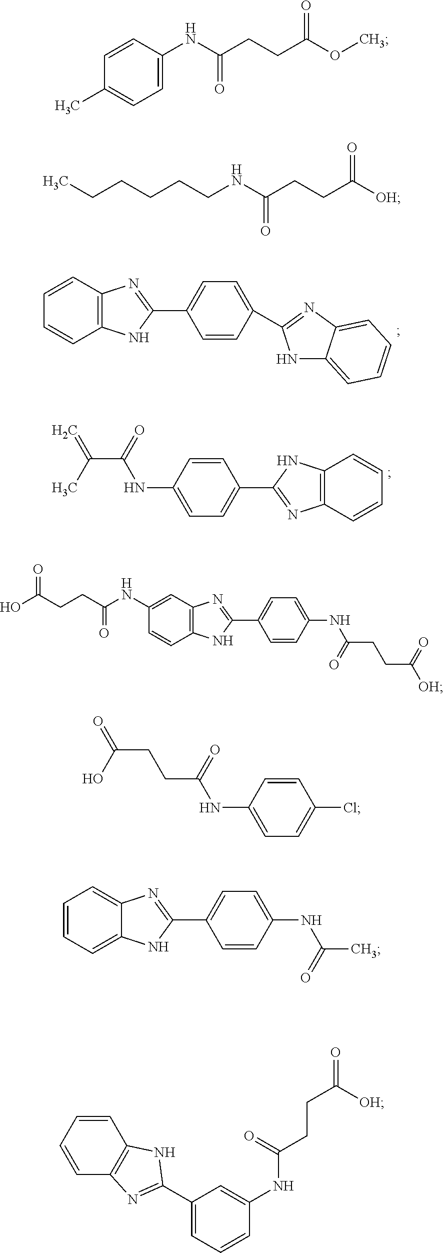

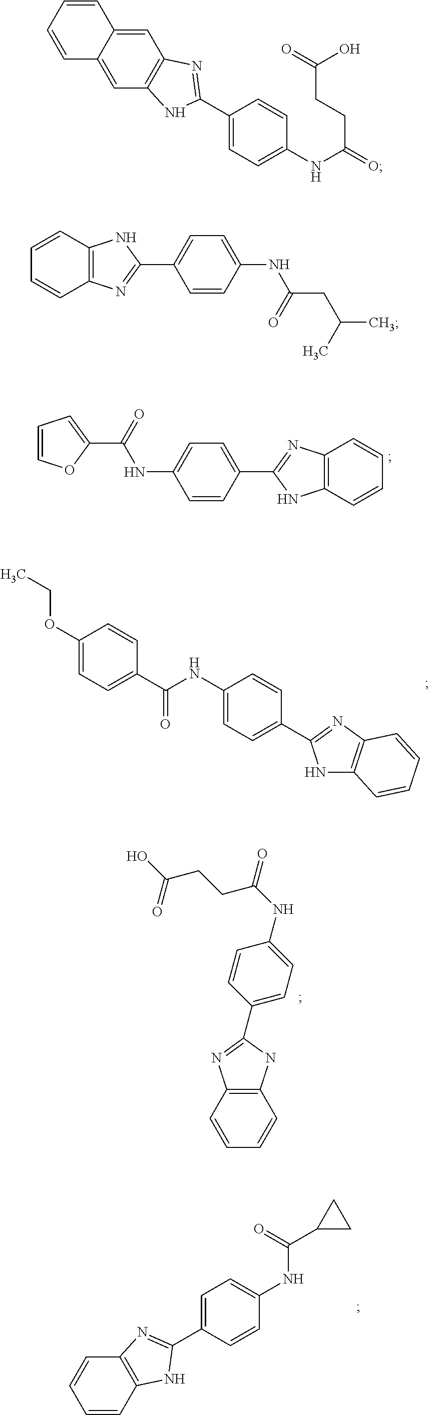 [SMILES: O=C(O)CCC(=O)Nc1ccc(-c2nc3cc4ccccc4cc3[nH]2)cc1 ; CC(C)CC(=O)Nc1ccc(-c2nc3ccccc3[nH]2)cc1 ; O=C(Nc1ccc(-c2nc3ccccc3[nH]2)cc1)c1ccco1 ; CCOc1ccc(C(=O)Nc2ccc(-c3nc4ccccc4[nH]3)cc2)cc1 ; O=C(O)CCC(=O)Nc1ccc(-c2nc3ccccc3[nH]2)cc1 ; O=C(Nc1ccc(-c2nc3ccccc3[nH]2)cc1)C1CC1]

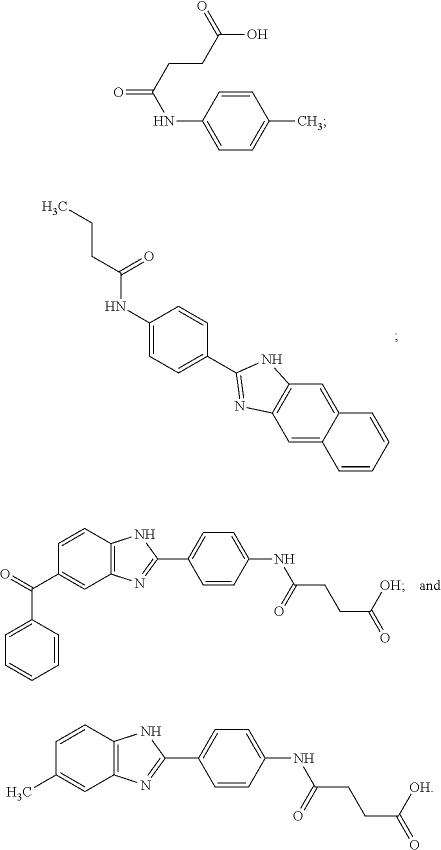 [SMILES: Cc1ccc(NC(=O)CCC(=O)O)cc1 ; CCCC(=O)Nc1ccc(-c2nc3cc4ccccc4cc3[nH]2)cc1 ; O=C(O)CCC(=O)Nc1ccc(-c2nc3cc(C(=O)c4ccccc4)ccc3[nH]2)cc1 ; "and" ; Cc1ccc2[nH]c(-c3ccc(NC(=O)CCC(=O)O)cc3)nc2c1]

19. The method of claim 1, wherein the compound reduces membrane potential by no more than 4%.

20. The method of claim 1, wherein the individual has or is suspected of having a disease or condition in which ROS is implicated.

21. The method of claim 20, wherein the disease or condition is selected from the group consisting of atherosclerosis, heart disease, heart failure, hypertension, sepsis, diabetes, Alzheimer's disease, Parkinson's disease, toxin-induced parkinsonism, Huntington's disease, Wilson's disease, Friedreich's Ataxia, Kearns-Sayre syndrome, Leigh syndrome, Leber hereditary optic neuropathy, mitochondrial myopathy, cardiomyopathy, deafness, mood disorders, movement disorders, dementia, Amyotropic Lateral Sclerosis, Multiple Sclerosis, tardive dyskinesia, brain injury, schizophrenia, epilepsy, AIDS dementia, endothelial nitroglycerin tolerance, adriamycin toxicity, kidney damage in type I diabetes, kidney preservation ex vivo, cocaine toxicity, alcohol fatty liver disease, fatty liver disease, liver inflammation in hepatitis C virus patients, neuroprotection, immobilization-induced muscle atrophy, skeletal muscle burn injury, cancer, inflammation and ischemic-reperfusion injury in stroke, heart attack, UV damage to the skin, and during organ transplantation and surgery.

22. The method of claim 21, wherein the individual has or is suspected of having the disease or condition selected from the group consisting of cancer, diabetes, an ischemia/reperfusion injury, Alzheimer's disease and chronic inflammation.

23. The method of claim 22, wherein the individual has or is suspected of having a cerebral or cardiac ischemia reperfusion injury.

24. The method of claim 23, wherein the individual has or is suspected of having a stroke or a heart-attack.

25. A pharmaceutical composition comprising a pharmaceutically acceptable carrier and
(A) a compound selected from the group consisting of: 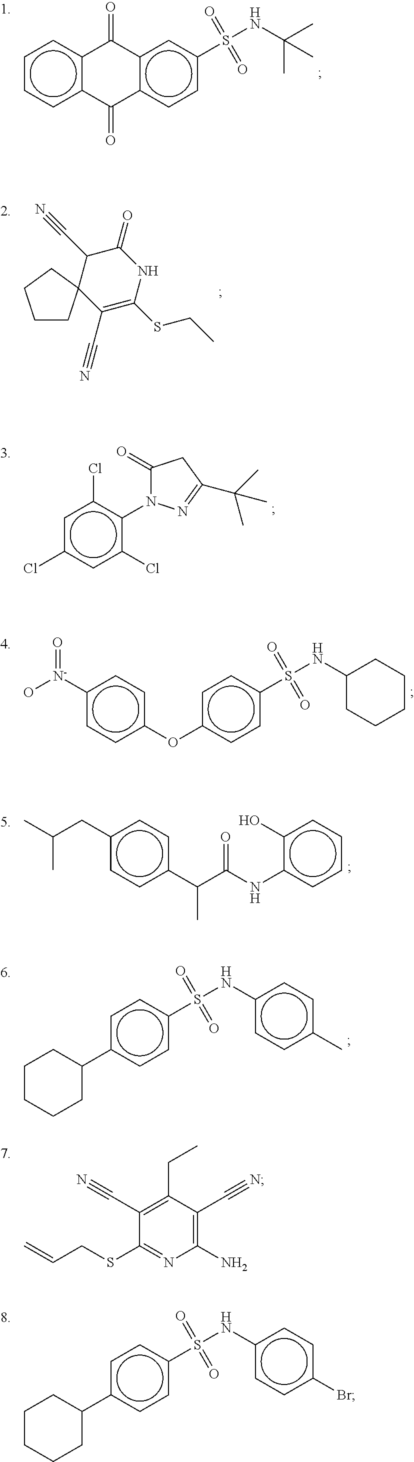 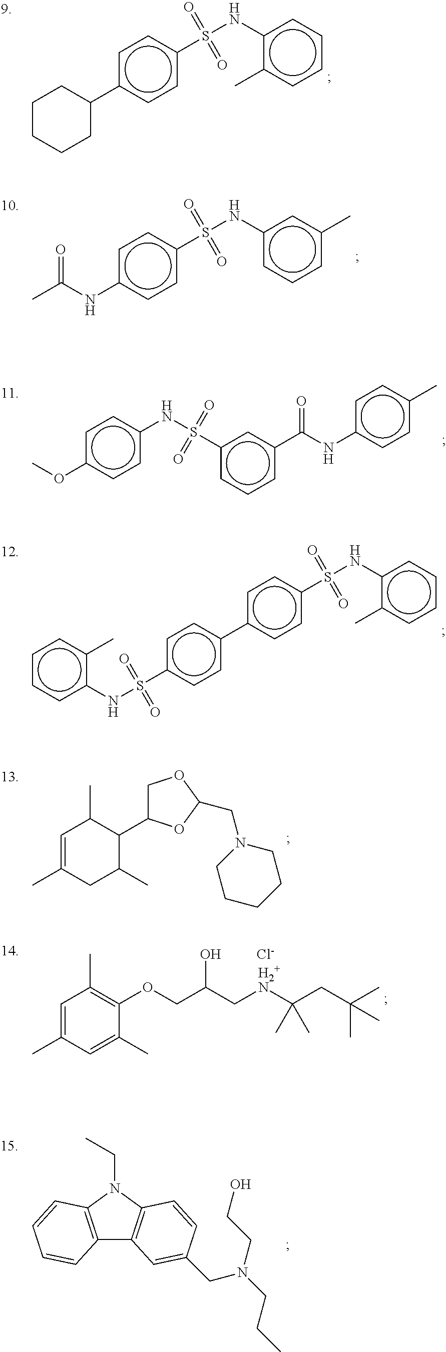 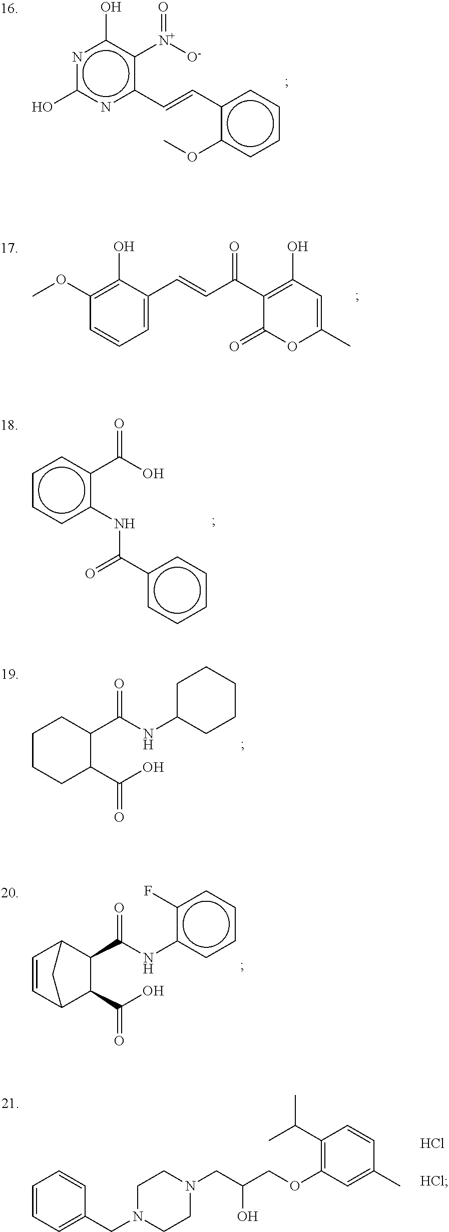 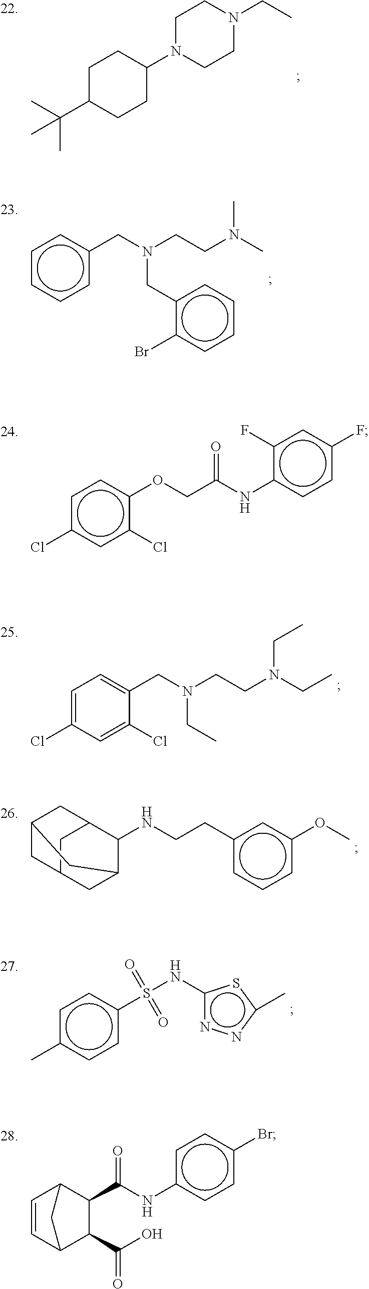 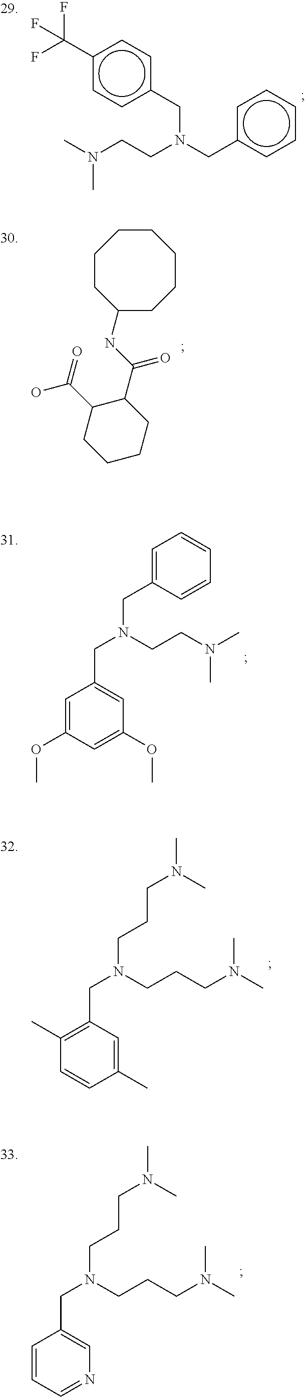 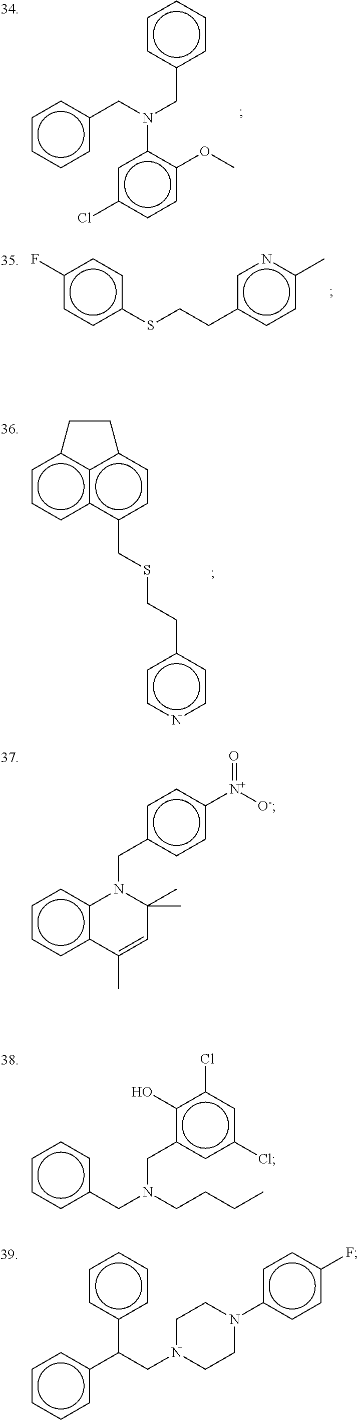 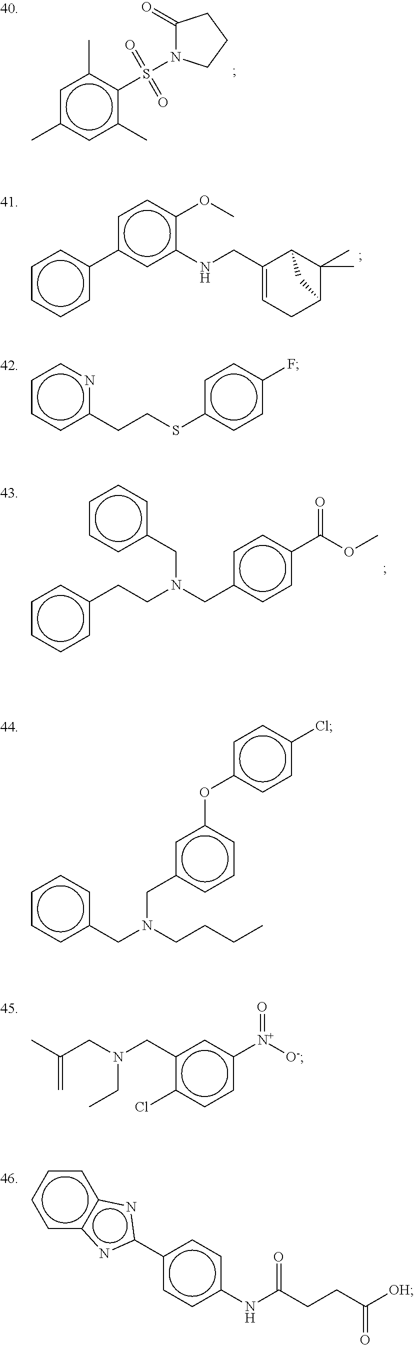 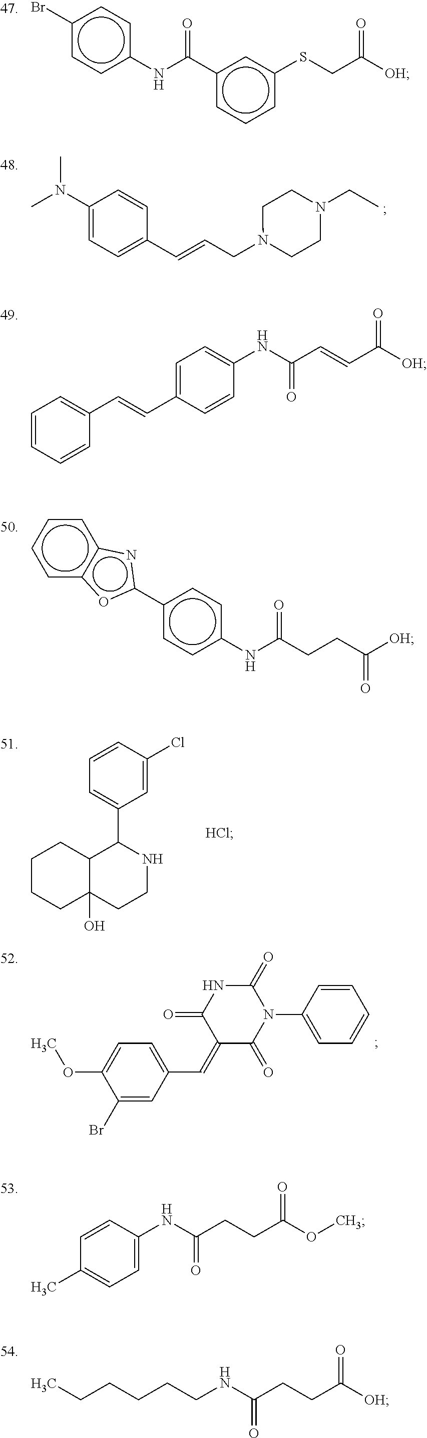 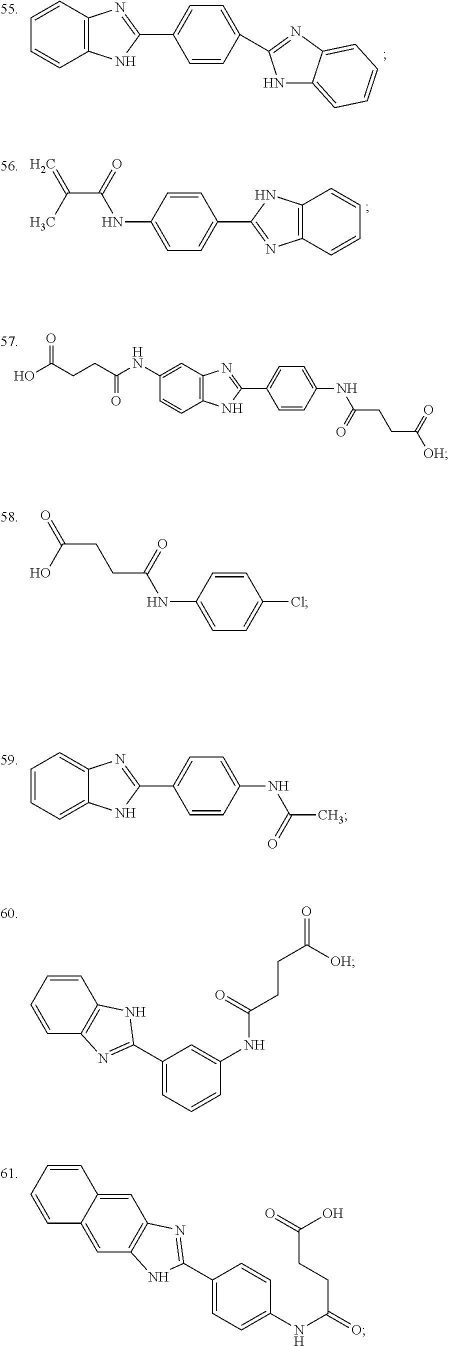 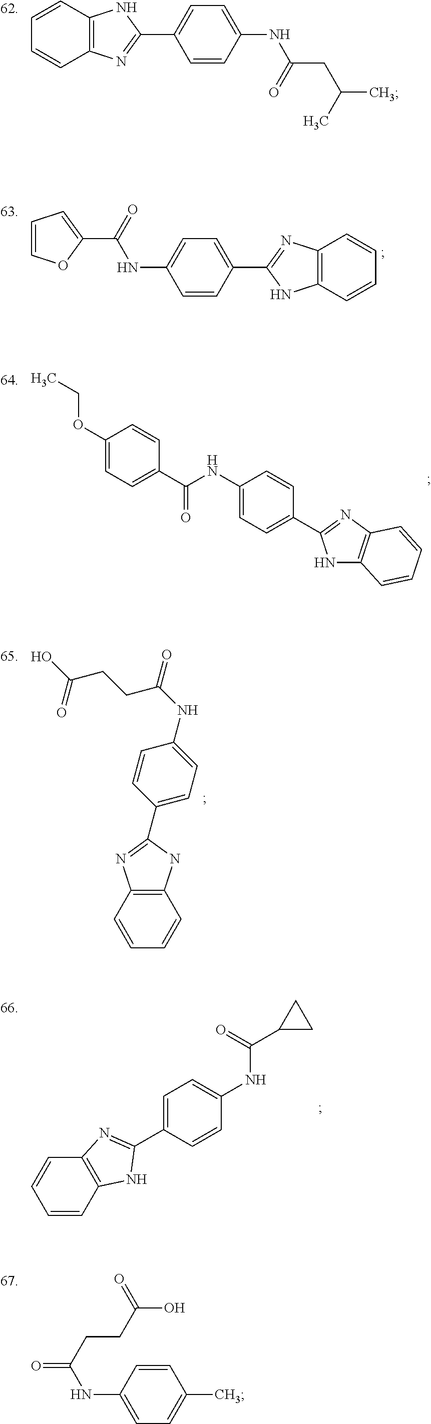 [SMILES: CC(C)CC(=O)Nc1ccc(-c2nc3ccccc3[nH]2)cc1 ; O=C(Nc1ccc(-c2nc3ccccc3[nH]2)cc1)c1ccco1 ; CCOc1ccc(C(=O)Nc2ccc(-c3nc4ccccc4[nH]3)cc2)cc1 ; O=C(O)CCC(=O)Nc1ccc(-c2nc3ccccc3[nH]2)cc1 ; O=C(Nc1ccc(-c2nc3ccccc3[nH]2)cc1)C1CC1 ; Cc1ccc(NC(=O)CCC(=O)O)cc1] 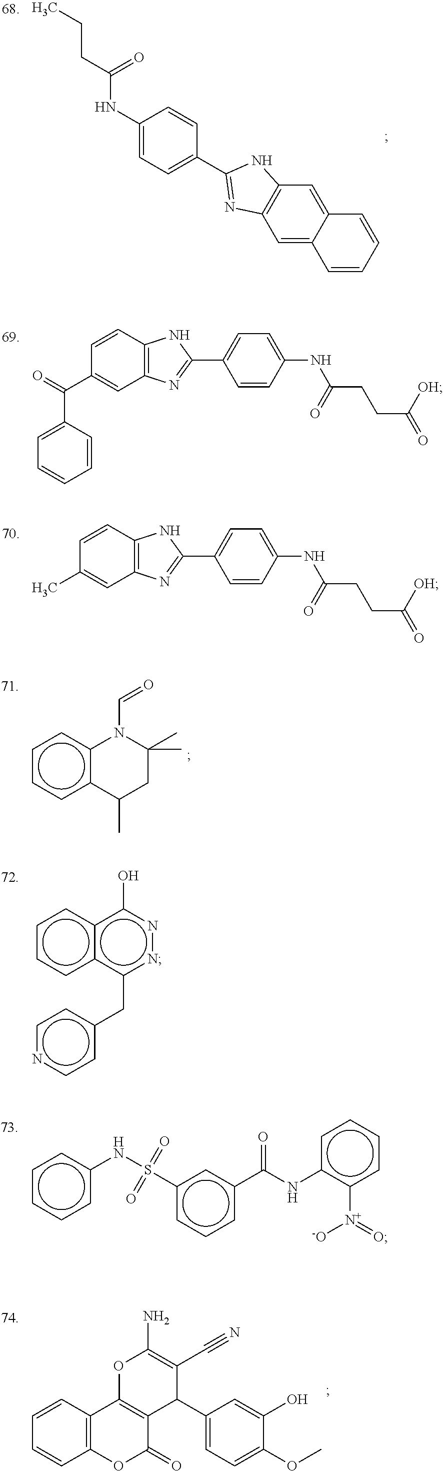 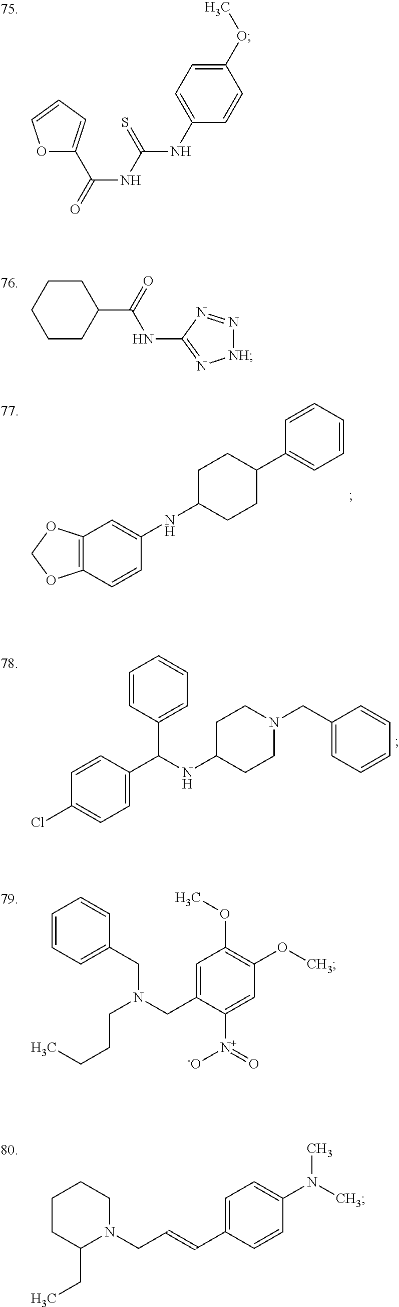 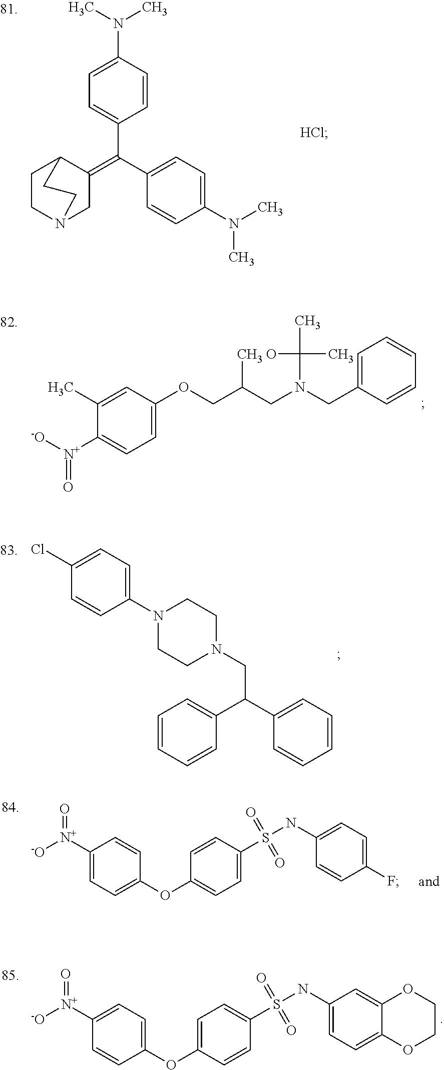
or
(B) a compound of the formula (I): 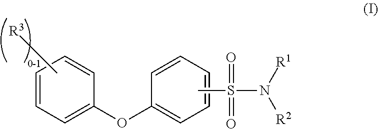
wherein:
R¹ and R² are independently selected from the group consisting of H, unsubstituted C₁-C₆ alkyl, —N═C(H)(aryl), —NH₂, unsubstituted aryl, substituted aryl or may be taken together with the nitrogen to which they are attached to form a nitrogen containing heterocyclic ring that may contain one additional heteroatom; and
R³ is selected from the group consisting of nitro, halo and C₁-C₆alkoxy;
or
(C) a compound of the formula (II): 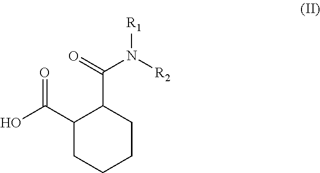
wherein R¹ and R² are independently H or a C₃-C₈cycloalkyl,
or
(D) a compound of the formula (III): 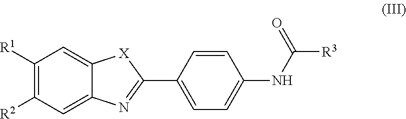
wherein:
R¹ and R² are independently selected from the group consisting of H, unsubstituted C₁-C₆ alkyl, C(═O)phenyl, phenyl, or may be taken together to form an aromatic ring;
R³ is selected from the group consisting of C₁-C₆ alkyl, carboxyl, and phenyl substituted with C₁-C₆ alkoxy; and
X is selected from the group NH, S and O,
or a pharmaceutically acceptable salt thereof.

26. A method of:
(A) identifying a compound capable of: (1) selectively inhibiting ROS production at one of the ROS production sites IQ, IF, IIIQ_{O}, SDH, or GPDH to a greater extent than the remaining ROS production sites IQ, IF, IIIQ_{O}, SDH, and GPDH and (2) decreasing mitochondrial membrane potential by no more than 4% relative to untreated mitochondria, wherein the method comprises the steps of contacting a cell or purified mitochondria with a compound and measuring ROS production at ROS production sites IQ, IF, IIIQ_{O}, SDH and GPDH and measuring membrane potential; or
(B) identifying a compound capable of selectively inhibiting ROS production at one of the ROS production sites IQ, IF, IIIQ_{O}, SDH, or GPDH to a greater extent than the remaining ROS production sites IQ, IF, IIIQ_{O}, SDH, and GPDH, wherein the method comprises the steps of contacting a cell or purified mitochondria with a compound and measuring ROS production at ROS production sites IQ, IF, IIIQ_{O}, SDH and GPDH; or
(C) identifying a compound capable of selectively inhibiting enzymatic activity of one of the ROS production sites IQ, IF, IIIQ_{O}, SDH, or GPDH to a greater extent than the remaining ROS production sites IQ, IF, IIIQ_{O}, SDH, and GPDH, wherein the method comprises the steps of contacting a cell or purified mitochondria with a compound and measuring ROS production at ROS production sites IQ, IF, IIIQ_{O}, SDH and GPDH; or
(D) identifying a compound capable of: (1) inhibiting ROS production at one or more ROS production sites IQ, IF, IIIQ_{O}, SDH, or GPDH and (2) decreasing mitochondrial membrane potential by no more than 4% relative to untreated mitochondria, wherein the method comprises the steps of contacting a cell or purified mitochondria with a compound and measuring ROS production at ROS production sites IQ, IF, IIIQ_{O}, SDH and GPDH and measuring membrane potential.

27. The method of claim 26, wherein the compound decreases ROS production from one of the ROS production sites IQ, IF, IIIQ_{O}, SDH, or GPDH by at least about 20% and decreases ROS production from the remaining ROS production sites IQ, IF, IIIQ_{O}, SDH, and GPDH by less than about 10% while decreasing mitochondrial membrane potential by no more than 4% relative to untreated mitochondria.
